(19) United States
(12) Patent Application Publication
(10) Pub. No.: US 2023/0382891 A1
(43) Pub. Date: Nov. 30, 2023

(54) GLUTAMINYL-PEPTIDE CYCLOTRANSFERASE LIKE (QPCTL) PROTEIN INHIBITORS AND USES THEREOF

(71) Applicant: Blacksmith Medicines, Inc., San Diego, CA (US)

(72) Inventors: Min TENG, San Diego, CA (US); David T. PUERTA, San Diego, CA (US); Baskar NAMMALWAR, San Diego, CA (US); Christian PEREZ, San Diego, CA (US); David LONERGAN, San Diego, CA (US); David Pifer MARTIN, San Diego, CA (US)

(21) Appl. No.: 18/249,465

(22) PCT Filed: Oct. 19, 2021
(86) PCT No.: PCT/US2021/055548

§ 371 Date: Apr. 18, 2023

Related U.S. Application Data
(60) Provisional application No. 63/253,310, filed on Oct. 7, 2021.
(60) Provisional application No. 63/094,210, filed on Oct. 20, 2020.
(60) Provisional application No. 63/094,202, filed on Oct. 20, 2020.

(51) Int. Cl. C07D 401/14; C07D 413/14; C07D 471/04

(52) U.S. Cl. CPC C07D 401/14; C07D 413/14; C07D 471/04

(57) ABSTRACT

Described herein are compounds that are glutaminyl-peptide cyclotransferase like (QPCTL) protein modulators, methods of making such compounds, pharmaceutical compositions and medicaments comprising such compounds, and methods of using such compounds in the treatment of conditions, diseases, or disorders that would benefit from modulation of QPCTL activity.

### CROSS-REFERENCE TO RELATED APPLICATIONS

This application claims the benefit of U.S. Provisional Patent Application No. 63/094,202, filed Oct. 20, 2020, U.S. Provisional Patent Application No. 63/094,210, filed Oct. 20, 2020, and U.S. Provisional Patent Application No. 63/253,310, filed Oct. 7, 2021, each of which is incorporated herein by reference in its entirety.

### BACKGROUND OF THE INVENTION

Glutaminyl-peptide cyclotransferase like (QPCTL) protein has emerged as a potential target for cancer immunotherapy. The inhibition of QPCTL prevents formation of pyroglutamate on CD47 at the SIRPα binding site, therefore interfering with the critical CD47 signaling pathway. Genetic and pharmacological interference with QPCTL activity enhances antibody-dependent cellular phagocytosis and cellular cytotoxicity of tumor cells, and interference with QPCTL expression leads to an increase in neutrophil-mediated killing of tumor cells in vivo.

Accordingly, QPCTL represents an attractive target for the treatment of conditions, diseases, or disorders that would benefit from modulating such activity, including cancer.

### SUMMARY OF THE INVENTION

Compounds described herein are glutaminyl-peptide cyclotransferase like (QPCTL) protein modulator compounds. In some embodiments, the compounds described herein are glutaminyl-peptide cyclotransferase like (QPCTL) protein modulator compounds. In some embodiments, the compounds described herein are glutaminyl-peptide cyclotransferase like (QPCTL) protein antagonists. In some embodiments, the compounds described herein are glutaminyl-peptide cyclotransferase like (QPCTL) protein inhibitors.

In one aspect, provided herein is a compound of Formula (A):

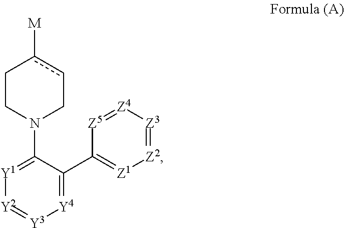

or a pharmaceutically acceptable salt, or solvate thereof, wherein:
M is

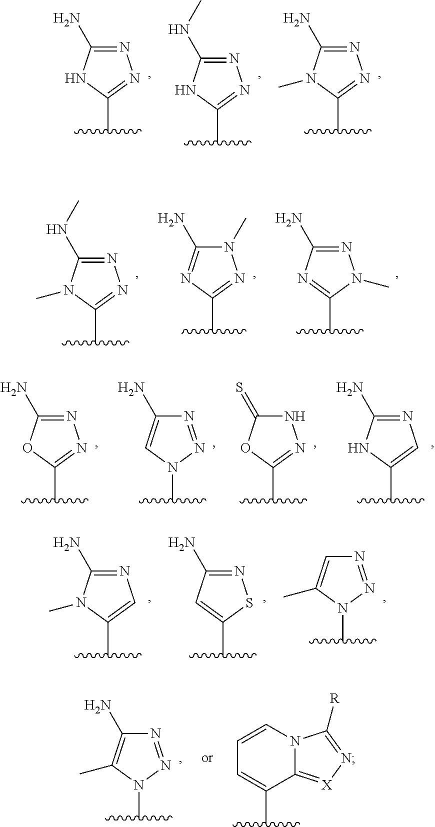

X is N or CH;
R is H, NH₂, or NHCH₃;
[symbol] is a single bond or a double bond;
Y¹ is N or CR¹; Y² is N or CR²; Y³ is N or CR³; and Y⁴ is N or CR⁴;
Z¹ is N or CR⁵; Z² is N or CR⁶; Z³ is N or CR⁷; Z⁴ is N or CR⁸; and Z⁵ is N or CR⁹;
R¹, R², R³, R⁴, R⁵, R⁶, R⁷, R⁸, and R⁹ are each independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, —CN, —OR¹⁰, —CO₂R¹⁰, —C(═O)N(R¹⁰)₂, —N(R¹⁰)₂, —NR¹⁰C(═O)R¹¹, —SR¹⁰, —S(═O)R¹¹, —SO₂R¹¹, or —SO₂N(R¹⁰)₂;
each R¹⁰ is independently selected from hydrogen, unsubstituted or substituted C₁-C₆ alkyl, unsubstituted or substituted C₁-C₆ heteroalkyl, unsubstituted or substituted C₃-C₆ cycloalkyl, 4- to 6-membered heterocycloalkyl;
or two R¹⁰ on the same N atom are taken together with the N atom to which they are attached to form an unsubstituted or substituted N-containing 4- to 6-membered heterocycloalkyl; and
each R¹¹ is independently selected from unsubstituted or substituted C₁-C₆ alkyl, unsubstituted or substituted C₁-C₆ heteroalkyl, unsubstituted or substituted C₃-C₆ cycloalkyl, 4- to 6-membered heterocycloalkyl;
wherein each substituted alkyl, substituted alkenyl, substituted alkynyl, substituted fluoroalkyl, substituted heteroalkyl, substituted cycloalkyl, and substituted heterocycloalkyl is substituted with one or more R^{s} groups independently selected from the group consisting of halogen, C₁-C₆ alkyl, monocyclic carbocycle, monocyclic heterocycle, —CN, —OR¹², —CO₂R¹², —C(═O)N(R¹²)₂, —N(R¹²)₂, —NR¹²C(═O)R¹³, —SR¹², —S(═O)R¹³, —SO₂R¹³, or —SO₂N(R¹³)₂;
   each R¹² is independently selected from hydrogen, C₁-C₆ alkyl, C₁-C₆ heteroalkyl, C₃-C₆ cycloalkyl, 4- to 6-membered heterocycloalkyl, phenyl, 5-membered heteroaryl and 6-membered heteroaryl; or two R¹² groups are taken together with the N atom to which they are attached to form a N-containing 4- to 6-membered heterocycloalkyl;
   each R¹³ is independently selected from C₁-C₆ alkyl, C₁-C₆ heteroalkyl, C₃-C₆ cycloalkyl, 4- to 6-membered heterocycloalkyl, phenyl, 5-membered heteroaryl and 6-membered heteroaryl;
wherein 0, 1, or 2 of Y¹, Y², Y³, and Y⁴ are N; and
wherein 0, 1, or 2 of Z¹, Z², Z³, Z⁴, and Z⁵ are N.

In some embodiments, the compound is a compound of Formula (A1):

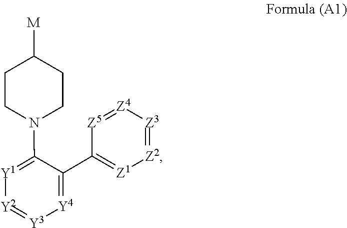

or a pharmaceutically acceptable salt, or solvate thereof.

In some embodiments, the compound is a compound of Formula (A2):

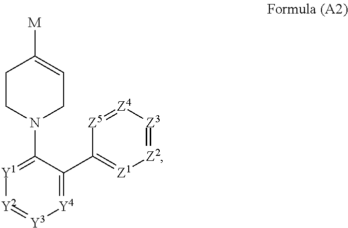

or a pharmaceutically acceptable salt, or solvate thereof.

In some embodiments, 0 or 1 of Y¹, Y², Y³, and Y⁴ is N. In some embodiments, 0 or 1 of Z¹, Z², Z³, Z⁴, and Z⁵ is N. In some embodiments, 0 or 1 of Y¹, Y², Y³, and Y⁴ is N; and 0 or 1 of Z¹, Z², Z³, Z⁴, and Z⁵ is N.

In some embodiments, the compound is a compound of Formula (B):

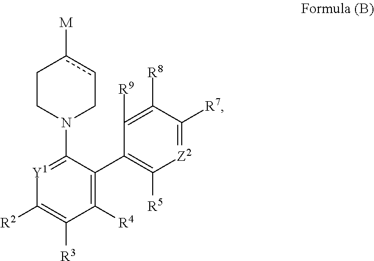

or a pharmaceutically acceptable salt, or solvate thereof.

In some embodiments, the compound is a compound of Formula (B1):

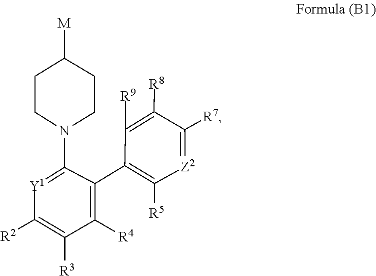

or a pharmaceutically acceptable salt, or solvate thereof.

In some embodiments, the compound is a compound of Formula (B2):

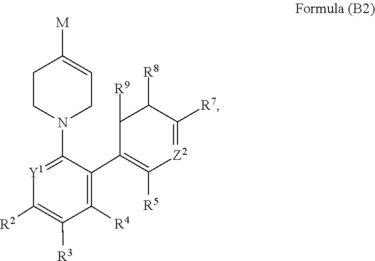

or a pharmaceutically acceptable salt, or solvate thereof.

In some embodiments, the compound is a compound of Formula (C):

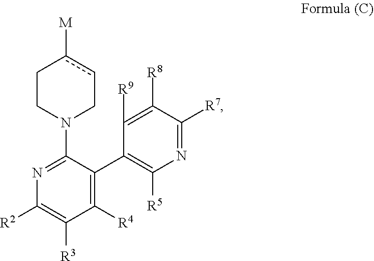

or a pharmaceutically acceptable salt, or solvate thereof.

In some embodiments, the compound is a compound of Formula (C1):

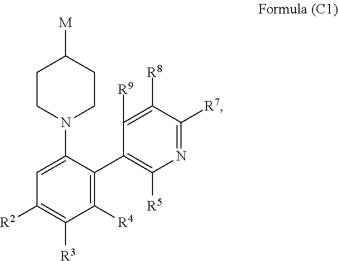

or a pharmaceutically acceptable salt, or solvate thereof.

In some embodiments, the compound is a compound of Formula (C2):

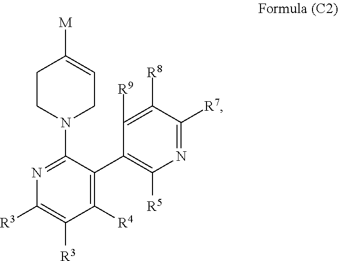

or a pharmaceutically acceptable salt, or solvate thereof.

In some embodiments, M is

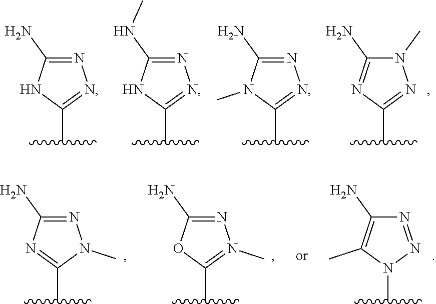

In some embodiments, M is

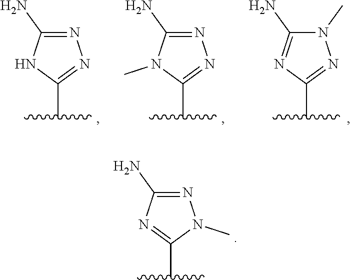

In some embodiments, M is

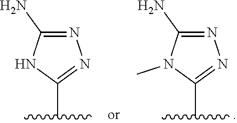

In some embodiments, the compound is a compound of Formula (C1-a) or Formula (C1-c):

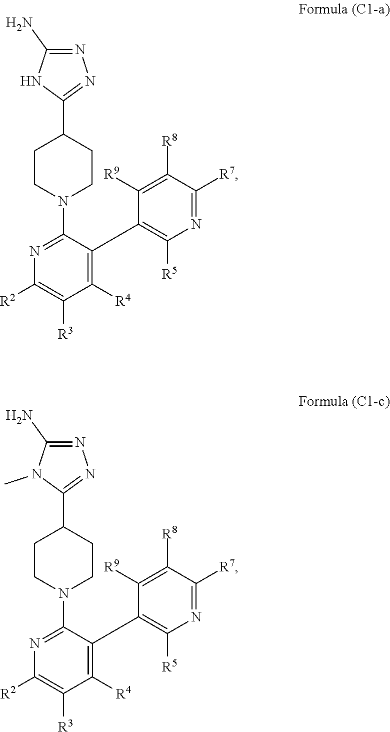

or a pharmaceutically acceptable salt, or solvate thereof.

In some embodiments, M is

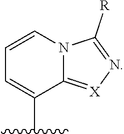

In some embodiments, X is N. In some embodiments, X is CH. In some embodiments, R is NH₂. In some embodiments, R is H.

In some embodiments, M is

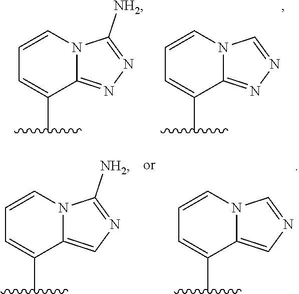

In some embodiments, M is or

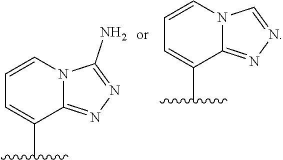 [SMILES: *c1cccn2c(N)nnc12 ; *c1cccn2cnnc12]

In some embodiments, the compound is a compound of Formula (C1-o):

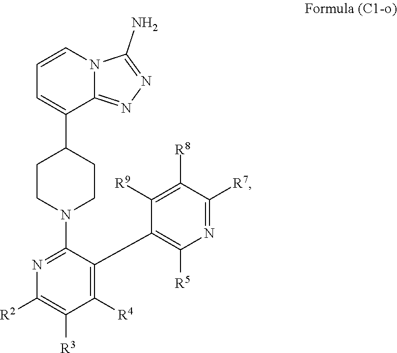

or a pharmaceutically acceptable salt, or solvate thereof.

In some embodiments, the compound is a compound of Formula (C2-o) or Formula (C2-p):

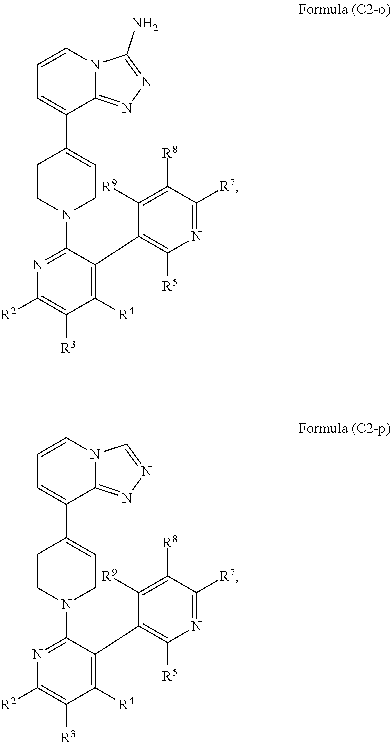

or a pharmaceutically acceptable salt, or solvate thereof.

In some embodiments, R¹, R², R³, R⁴, R⁵, R⁶, R⁷, R⁸, and R⁹ are each independently hydrogen, halogen, —CN, unsubstituted or substituted C₁-C₆ alkyl, or unsubstituted or substituted C₁-C₆ fluoroalkyl. In some embodiments, R², R³, R⁴, R⁵, R⁷, R⁸, and R⁹ are each independently hydrogen, —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃.

In some embodiments, R², R³, R⁴, R⁵, R⁷, R⁸, and R⁹ are each independently hydrogen, —F, or —CH₃.

In some embodiments, the compound is a compound of Formula (D):

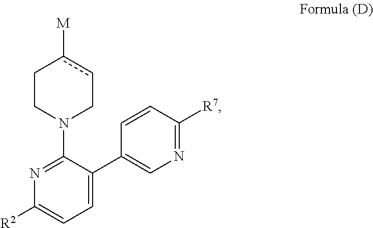

or a pharmaceutically acceptable salt, or solvate thereof. In some embodiments, R² and R⁷ are each independently hydrogen, halogen, —CN, —OR¹⁰, —N(R¹⁰)₂, C₁-C₆ alkyl, or C₁-C₆ fluoroalkyl.

In some embodiments, the compound is a compound of Formula (D1):

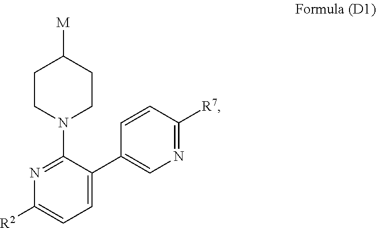

or a pharmaceutically acceptable salt, or solvate thereof. In some embodiments, R² and R⁷ are each independently hydrogen, halogen, —CN, —OR¹⁰, —N(R¹⁰)₂, C₁-C₆ alkyl, or C₁-C₆ fluoroalkyl.

In some embodiments, the compound is a compound of Formula (D2):

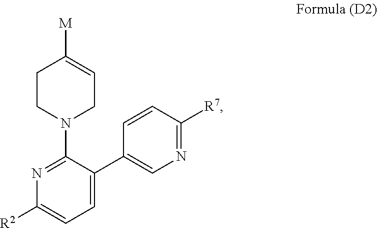

or a pharmaceutically acceptable salt, or solvate thereof. In some embodiments, R² and R⁷ are each independently hydrogen, halogen, —CN, —OR¹⁰, —N(R¹⁰)₂, C₁-C₆ alkyl, or C₁-C₆ fluoroalkyl.

In some embodiments, R² is hydrogen, —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃; and R⁷ is hydrogen, —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃. In some embodiments, R² is hydrogen, —F, or —CH₃; R⁷ is hydrogen, —F, —Cl, —CN, —OCH₃, —OCH₂CH₂OCH₃, or —NH₂.

Any combination of the groups described above for the various variables is contemplated herein. Throughout the specification, groups and substituents thereof are chosen by one skilled in the field to provide stable moieties and compounds.

Also described herein is a pharmaceutical composition comprising a compound described herein, or a pharmaceutically acceptable salt, or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

Also described herein is a method of treating a disease or condition in a mammal that would benefit from the modulation of glutaminyl-peptide cyclotransferase like (QPCTL) protein activity comprising administering a compound described herein, or pharmaceutically acceptable salt, or solvate thereof, to the mammal in need thereof. In some embodiments, the disease or condition is cancer. In some embodiments, the cancer is a leukemia or lymphoma. In some embodiments, the leukemia or lymphoma is acute myeloid leukemia (AML), chronic myeloid leukemia (CMIL), acute lymphocytic leukemia (ALL), chronic lymphocytic leukemia (CLL), non-Hodgkin's lymphoma (NHL). In some embodiments, the non-Hodgkin's lymphoma is a B-cell lymphoma. In some embodiments, the B-cell lymphoma is selected from the group consisting of Burkitt lymphoma, hairy cell lymphoma (HCL), Waldenstrom macroglobulinemia, chronic lymphocytic leukemia/small lymphocytic lymphoma (CLL/SLL), diffuse large B cell lymphoma (DLBCL), B cell chronic lymphocytic leukemia (B-CLL), mantle cell lymphoma (MCL), follicular lymphoma (FL), marginal zone lymphoma (MZL), and pre-B acute lymphoblastic leukemia (pre-B ALL). In some embodiments, the cancer is selected from the group consisting of: multiple myeloma (MM), ovarian cancer, gliomas, colon cancer, breast cancer, bladder cancer, gastric cancer, esophageal cancer, small cell lung cancer (SCLC), non-small cell lung cancer (NSCLC), head and neck squamous cell cancer, mesothelioma, melanoma, glioma, glioblastoma, and pancreatic neuroendocrine tumors.

In any of the aforementioned aspects are further embodiments in which the effective amount of the compound described herein, or a pharmaceutically acceptable salt, or solvate thereof, is: (a) systemically administered to the mammal; and/or (b) administered orally to the mammal; and/or (c) intravenously administered to the mammal; and/or (d) administered by inhalation; and/or (e) administered by nasal administration; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

In any of the embodiments disclosed herein, the mammal is a human.

In some embodiments, compounds provided herein are orally administered to a human.

Articles of manufacture, which include packaging material, a compound described herein, or a pharmaceutically acceptable salt thereof, within the packaging material, and a label that indicates that the compound or composition, or pharmaceutically acceptable salt, tautomers, pharmaceutically acceptable N-oxide, pharmaceutically active metabolite, pharmaceutically acceptable prodrug, or pharmaceutically acceptable solvate thereof, is used for modulating glutaminyl-peptide cyclotransferase like (QPCTL) protein, or for the treatment, prevention or amelioration of one or more symptoms of a disease or condition that would benefit from modulating glutaminyl-peptide cyclotransferase like (QPCTL) protein, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Glutaminyl cyclase (QC, EC 2.3.2.5) (also known as glutaminyl-peptide cyclotransferase; QPCT) catalyzes the intramolecular cyclization of N-terminal glutamine residues into pyroglutamic acid (pGlu*) liberating ammonia. QPCT is primarily expressed in neuroendocrine tissues, such as the pituitary, and has been implicated in diseases and conditions such as Alzheimer's disease, Familial British dementia, Huntington's disease, Down syndrome, atherosclerosis, and rheumatoid arthritis. Iso glutaminyl cyclase (isoQC, also known as glutaminyl-peptide cyclotransferase like protein; QPCTL) is a related protein that differs in amino acid sequence, but performs much of the same catalytic activity as glutaminyl cyclase. However, in contrast to QPCT, QPCTL is expressed ubiquitously, and is especially abundant in peripheral blood lymphocytes and other blood cells.

The CD47/SIRP-alpha (SIRPα) interaction is an important myeloid immune checkpoint whose clinical relevance has been shown by successful application of CD47 antibodies in cancer therapy. CD47 (Cluster of Differentiation 47); also known as integrin associated protein (IAP)) is expressed on cancer cells and the ligand SIRPα (Signal regulatory protein α) is expressed on myeloid cells like macrophages and NK cells. Engagement of SIRPα leads to inhibition of phagocytosis. Accordingly, high levels of CD47 allow cancer cells to avoid phagocytosis due to engagement of the SIRPα of macrophages. Thus, blocking CD47/SIRPα interactions turns off the “don't eat me” signal and favors phagocytosis. QPCTL is critical for pyroglutamate formation on the N-terminus of CD47 shortly after biosynthesis, which is essential for the binding of CD47 to SIRPα. Accordingly, QPCTL is an attractive target to silence the “don't eat me” signal provided by the CD47/SIRPα interaction.

While there are antibody approaches in clinical development for anti-CD47 therapy, small molecule QPCTL modulators represent an attractive therapeutic approach for cancer immunotherapy. Antibodies are typically polar, heat sensitive, membrane impermeable, and subject to enzymatic degradation, and are, accordingly, not orally available and must be administered systemically though, for example, IV injection. In contrast, small molecule QPCTL modulators are easier and cheaper to produce than antibody therapies, and can be administered by a variety of routes, including oral administration.

The CCL2/CCR2 axis is an important chemokine signaling axis in the recruitment of myeloid lineage cells with clinical significance in various cancer and inflammatory or autoimmune disorders. The N-terminus of monocyte chemoattractant protein 1 (MCP-1, aka CCL2) is a modified to a pyroglutamate (pGlu*) residue. QPCTL has been shown to have a major role in the pyroglutamate formation on CCL2, as well as monocyte infiltration. Current strategies targeting CCL2 are based on antibodies. Accordingly, small, orally available inhibitors of QPCTL represents an alternative therapeutic strategy to treat CCL2-driven disorders.

In some embodiments, the QPCTL modulators described herein have utility over a wide range of therapeutic applications.

In some embodiments, the QPCTL modulators described herein are used in the treatment of a variety of diseases or conditions such as, but not limited to, cancer, inflammatory disorders, atherosclerosis/restenosis, and fibrosis.

In some embodiments, the QPCTL modulators described herein are used in the treatment of cancer. In some embodiments, the cancer is a blood cancer, such as, but not limited to, a lymphoma or leukemia. In some embodiments, the leukemia or lymphoma is acute myeloid leukemia (AML), chronic myeloid leukemia (CML), acute lymphocytic leukemia (ALL), chronic lymphocytic leukemia (CLL), non-Hodgkin's lymphoma (NHL). In some embodiments, the non-Hodgkin's lymphoma is a B-cell lymphoma. In some embodiments, the B-cell lymphoma is selected from the group consisting of Burkitt lymphoma, hairy cell lymphoma (HCL), Waldenstrom macroglobulinemia, chronic lymphocytic leukemia/small lymphocytic lymphoma (CLL/SLL), diffuse large B cell lymphoma (DLBCL), B cell chronic lymphocytic leukemia (B-CLL), mantle cell lymphoma (MCL), follicular lymphoma (FL), marginal zone lymphoma (MZL), and pre-B acute lymphoblastic leukemia (pre-B ALL). In some embodiments, the cancer is selected from the group consisting of: multiple myeloma (MM), ovarian cancer, gliomas, colon cancer, breast cancer, bladder cancer, gastric cancer, esophageal cancer, small cell lung cancer (SCLC), non-small cell lung cancer (NSCLC), head and neck squamous cell cancer, mesothelioma, melanoma, glioma, glioblastoma, and pancreatic neuroendocrine tumors.

In some embodiments, the cancer is a skin, tissue, or bone cancer, such as, but not limited to, a carcinoma, sarcoma, or melanoma. In some embodiments, the carcinoma, sarcoma, or melanoma is basal cell carcinoma, squamous cell carcinoma, renal cell carcinoma, invasive ductal carcinoma, adenocarcinoma, Merkel cell carcinoma, skin cancer, lung cancer, breast cancer, prostate cancer, colorectal cancer, soft tissue sarcoma, osteosarcoma, Ewing's sarcoma, chrondrosarcoma, myeloma, or multiple myeloma.

In some embodiments, the QPCTL modulators described herein are used in the treatment of a cancer which overexpresses CD47. In some embodiments, the cancer expresses CD47 in a diseased cell at 1.5 times, 2 times, 2.5 times, 3 times, or more of the level of a non-diseased cell of the same type.

In some embodiments, the QPCTL modulators described herein are used in the treatment of cancer in a mammal.

In some embodiments, the QPCTL modulators described herein are used in the treatment of diseases and conditions in which CCL2-CCR2 signaling plays a role. In some embodiments, the QPCTL modulators described herein are used in the treatment of diseases and conditions involving the recruitment of myeloid lineage cells. In some embodiments, the disease or condition is an inflammatory disease, autoimmune disease, allergic inflammatory disease, or neurodegenerative disease, such as, but not limited to, rheumatoid arthritis, inflammatory bowel disease, multiple sclerosis, psoriasis, psoriatic arthritis, atopic dermatitis, severe asthma, allergic rhinitis and rhinosinusitis, nasal polyposis, atherosclerosis, pulmonary arterial hypertension, non-alcoholic steatohepatitis, chronic obstructive pulmonary disease, idiopathic pulmonary fibrosis, endometriosis, Alzheimer's disease and related dementias, Parkinson's disease, and Huntington's disease.

In some embodiments, the QPCTL modulators described herein are used in the treatment of a disease or condition in a mammal.

### Compounds

Compounds described herein, for example compounds of Formula (A), Formula (I), and Formula (XI), including pharmaceutically acceptable salts, prodrugs, active metabolites, and pharmaceutically acceptable solvates thereof, are glutaminyl-peptide cyclotransferase like (QPCTL) protein modulators. In some embodiments, the compounds described herein, including pharmaceutically acceptable salts, prodrugs, active metabolites, and pharmaceutically acceptable solvates thereof, are glutaminyl-peptide cyclotransferase like (QPCTL) protein antagonists. In some embodiments, the compounds described herein, including pharmaceutically acceptable salts, prodrugs, active metabolites, and pharmaceutically acceptable solvates thereof, are glutaminyl-peptide cyclotransferase like (QPCTL) protein inhibitors.

In one aspect, provided herein is a compound of Formula (A):

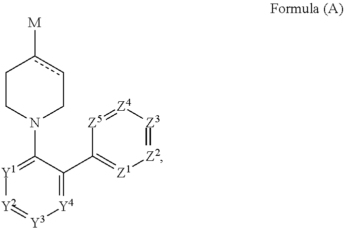

or a pharmaceutically acceptable salt, or solvate thereof, wherein:
M is

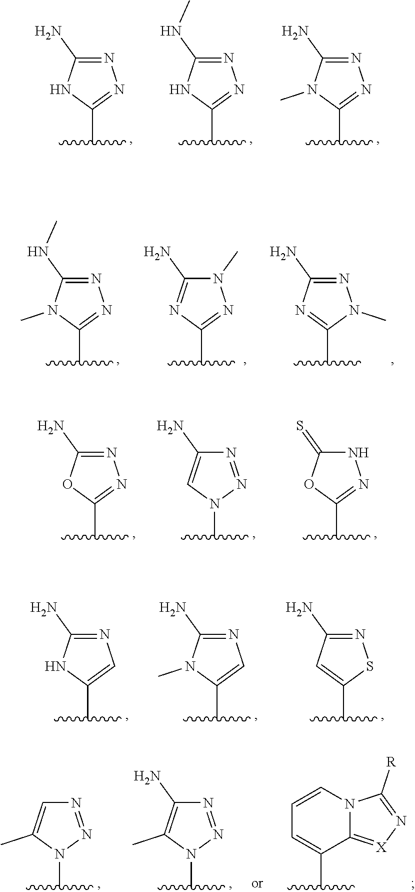

X is N or CH;
R is H, NH₂, or NHCH₃;
[symbol] is a single bond or a double bond;
Y¹ is N or CR¹; Y² is N or CR², Y³ is N or CR³, and Y⁴ is N or CR⁴;
Z¹ is N or CR⁵; Z² is N or CR⁶; Z³ is N or CR⁷; Z⁴ is N or CR⁸; and Z⁵ is N or CR⁹;
R¹, R², R³, R⁴, R⁵, R⁶, R⁷, R⁸, and R⁹ are each independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, —CN, —OR¹⁰, —CO₂R¹⁰, —C(═O)N(R¹⁰)₂, —N(R¹⁰)₂, —NR¹⁰C(═O)R¹¹, —SR¹⁰, —S(═O)R¹¹, —SO₂R¹¹, or —SO₂N(R¹⁰)₂;
each R¹⁰ is independently selected from hydrogen, unsubstituted or substituted C₁-C₆ alkyl, unsubstituted or substituted C₁-C₆ heteroalkyl, unsubstituted or substituted C₃-C₆ cycloalkyl, 4- to 6-membered heterocycloalkyl;
or two R¹⁰ on the same N atom are taken together with the N atom to which they are attached to form an unsubstituted or substituted N-containing 4- to 6-membered heterocycloalkyl; and
each R¹¹ is independently selected from unsubstituted or substituted C₁-C₆ alkyl, unsubstituted or substituted C₁-C₆ heteroalkyl, unsubstituted or substituted C₃-C₆ cycloalkyl, 4- to 6-membered heterocycloalkyl;
wherein each substituted alkyl, substituted alkenyl, substituted alkynyl, substituted fluoroalkyl, substituted heteroalkyl, substituted cycloalkyl, and substituted heterocycloalkyl is substituted with one or more R^{s} groups independently selected from the group consisting of halogen, C₁-C₆ alkyl, monocyclic carbocycle, monocyclic heterocycle, —CN, —OR¹², —CO₂R¹², —C(═O)N(R¹²)₂, —N(R¹²)₂, —NR¹²C(═O)R¹³, —SR¹², —S(═O)R¹³, —SO₂R¹³, or —SO₂N(R¹³)₂;
   each R¹² is independently selected from hydrogen, C₁-C₆ alkyl, C₁-C₆ heteroalkyl, C₃-C₆ cycloalkyl, 4- to 6-membered heterocycloalkyl, phenyl, 5-membered heteroaryl and 6-membered heteroaryl; or two R¹² groups are taken together with the N atom to which they are attached to form a N-containing 4- to 6-membered heterocycloalkyl;
   each R¹³ is independently selected from C₁-C₆ alkyl, C₁-C₆ heteroalkyl, C₃-C₆ cycloalkyl, 4- to 6-membered heterocycloalkyl, phenyl, 5-membered heteroaryl and 6-membered heteroaryl;
wherein 0, 1, or 2 of Y¹, Y², Y³, and Y⁴ are N; and
wherein 0, 1, or 2 of Z¹, Z², Z³, Z⁴, and Z⁵ are N.

For any and all of the embodiments, substituents are selected from among a subset of the listed alternatives. For example, in some embodiments, [symbol] is a single bond. In other embodiments, [symbol] is a double bond.

In some embodiments, the compound is a compound of Formula (A1):

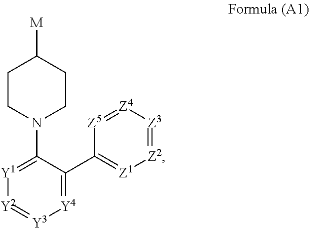

or a pharmaceutically acceptable salt, or solvate thereof.

In some embodiments, the compound is a compound of Formula (A2):

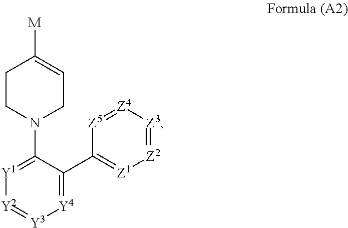

or a pharmaceutically acceptable salt, or solvate thereof.

In some embodiments, 0 or 1 of Y¹, Y², Y³, and Y⁴ is N. In some embodiments, 0 of Y¹, Y², Y³, and Y⁴ is N. In some embodiments, 1 of Y¹, Y², Y³, and Y⁴ is N.

In some embodiments, Y¹ is N or CR¹. In some embodiments, Y² is N or CR². In some embodiments, Y³ is N or CR³. In some embodiments, Y⁴ is N or CR⁴. In some embodiments, Y¹ is N. In some embodiments, Y¹ is CR¹. In some embodiments, Y² is N. In some embodiments, Y² is CR². In some embodiments, Y³ is N. In some embodiments, Y³ is CR³. In some embodiments, Y⁴ is N. In some embodiments, Y⁴ is CR⁴.

In some embodiments, Y¹ is CR¹; Y² is CR²; Y³ is CR³; and Y⁴ is CR⁴. In some embodiments, Y¹ is N; Y² is CR²; Y³ is CR³; and Y⁴ is CR⁴. In some embodiments, Y¹ is CR¹; Y² is N; Y³ is CR³; and Y⁴ is CR⁴. In some embodiments, Y¹ is CR¹; Y² is CR²; Y³ is N; and Y⁴ is CR⁴. In some embodiments, Y¹ is CR¹; Y² is CR²; Y³ is CR³; and Y⁴ is N.

In some embodiments, 0 or 1 of Z¹, Z², Z³, Z⁴, and Z⁵ is N. In some embodiments, 0 of Z¹, Z², Z³, Z⁴, and Z⁵ is N. In some embodiments, 1 of Z¹, Z², Z³, Z⁴, and Z⁵ is N.

In some embodiments, Z¹ is N or CR⁵. In some embodiments, Z² is N or CR⁶. In some embodiments, Z³ is N or CR⁷. In some embodiments, Z⁴ is N or CR⁸. In some embodiments, Z⁵ is N or CR⁹. In some embodiments, Z² is N. In some embodiments, Z² is CR⁶.

In some embodiments, Z¹ is CR⁵; Z² is CR⁶; Z³ is CR⁷; Z⁴ is CR⁸; and Z⁵ is CR⁹. In some embodiments, Z¹ is N; Z² is CR⁶; Z³ is CR⁷; Z⁴ is CR⁸; and Z⁵ is CR⁹. In some embodiments, Z¹ is CR⁵; Z² is N; Z³ is CR⁷; Z⁴ is CR⁸; and Z⁵ is CR⁹. In some embodiments, Z¹ is CR⁵; Z² is CR⁶; Z³ is N; Z⁴ is CR⁸; and Z⁵ is CR⁹. In some embodiments, Z¹ is CR⁵; Z² is CR⁶; Z³ is CR⁷; Z⁴ is N; and Z⁵ is CR⁹. In some embodiments, Z¹ is CR⁵; Z² is CR⁶; Z³ is CR⁷; Z⁴ is CR⁸; and Z⁵ is N.

In some embodiments, 0 or 1 of Y¹, Y², Y³, and Y⁴ is N; and 0 or 1 of Z¹, Z², Z³, Z⁴, and Z⁵ is N. In some embodiments, 0 of Y¹, Y², Y³, and Y⁴ is N; and 0 of Z¹, Z², Z³, Z⁴, and Z⁵ is N. In some embodiments, 0 of Y¹, Y², Y³, and Y⁴ is N; and 1 of Z¹, Z², Z³, Z⁴, and Z⁵ is N. In some embodiments, 1 of Y¹, Y², Y³, and Y⁴ is N; and 0 of Z¹, Z², Z³, Z⁴, and Z⁵ is N. In some embodiments, 1 of Y¹, Y², Y³, and Y⁴ is N; and 1 of Z¹, Z², Z³, Z⁴, and Z⁵ is N. In some embodiments, 1 of Y¹ and Y³ is N; and 1 of Z² and Z⁴ is N. In some embodiments, Y¹ is N; and 1 of Z² and Z⁴ is N. In some embodiments, 1 of Y¹ and Y³ is N; and Z² is N.

In some embodiments, the compound is a compound of Formula (B):

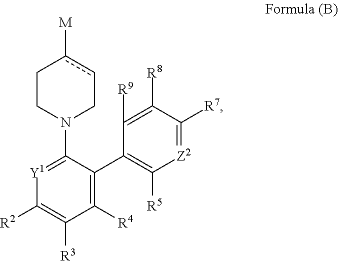

or a pharmaceutically acceptable salt, or solvate thereof.

In some embodiments, the compound is a compound of Formula (B1):

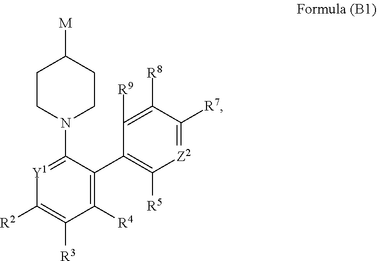

or a pharmaceutically acceptable salt, or solvate thereof.

In some embodiments, the compound is a compound of Formula (B2):

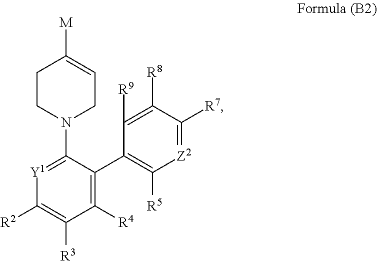

or a pharmaceutically acceptable salt, or solvate thereof.

In some embodiments, Y¹ is N; and Z² is N or CR⁶. In some embodiments, Y¹ is N; and Z² is CR⁶. In some embodiments, Y¹ is N; and Z² is N. In some embodiments, Y¹ is CR¹; and Z² is N or CR⁶. In some embodiments, Y¹ is CR¹; and Z² is CR⁶. In some embodiments, Y¹ is CR¹; and Z² is N.

In some embodiments, the compound is a compound of Formula (C):

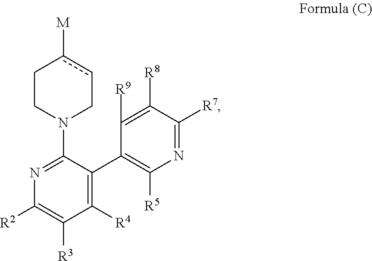

or a pharmaceutically acceptable salt, or solvate thereof.

In some embodiments, the compound is a compound of Formula (C1):

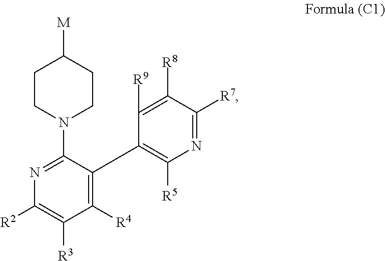

or a pharmaceutically acceptable salt, or solvate thereof.

In some embodiments, the compound is a compound of Formula (C2):

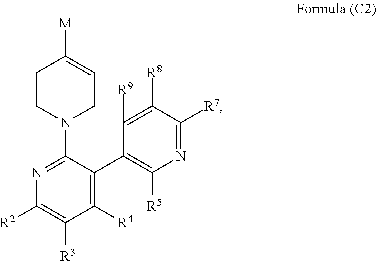

or a pharmaceutically acceptable salt, or solvate thereof.

In some embodiments, M is

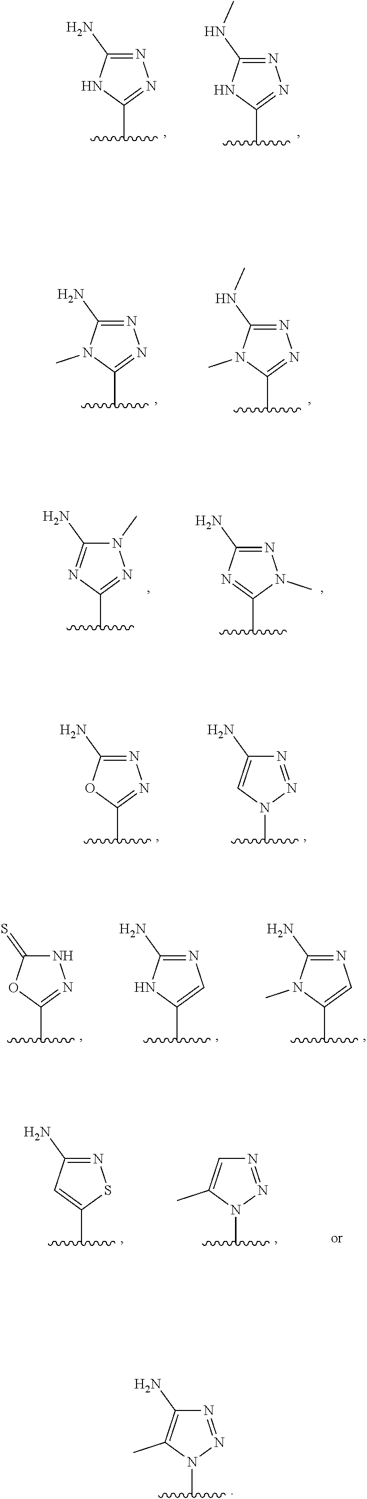

In some embodiments, M is

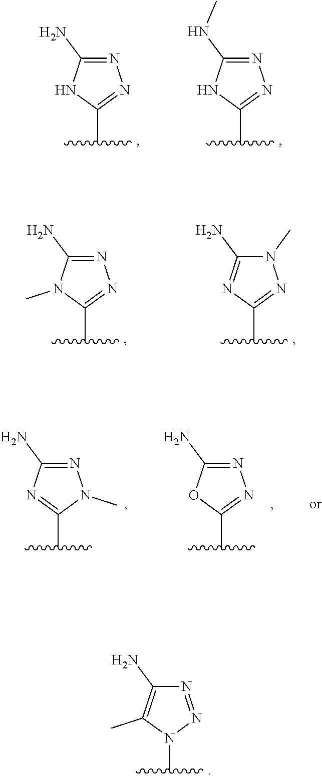

In some embodiments, M is

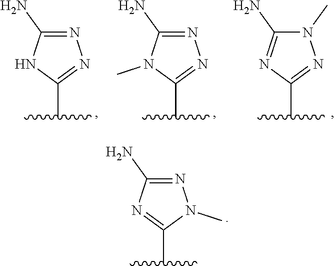

In some embodiments, M is

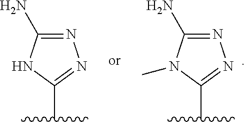

In some embodiments, M is

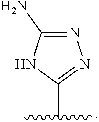 [SMILES: *c1nnc(N)[nH]1]

In some embodiments, M is

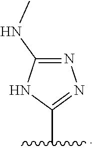 [SMILES: *c1nnc(NC)[nH]1]

In some embodiments, M is

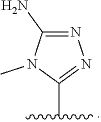 [SMILES: *c1nnc(N)n1C]

In some embodiments, M is

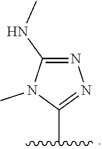 [SMILES: *c1nnc(NC)n1C]

In some embodiments, M is

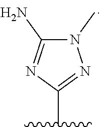 [SMILES: *c1nc(N)n(C)n1]

In some embodiments, M is

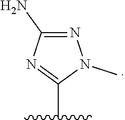 [SMILES: *c1nc(N)nn1C]

In some embodiments, M is

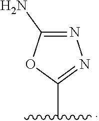 [SMILES: *c1nnc(N)o1]

In some embodiments, M is

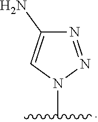 [SMILES: *n1cc(N)nn1]

In some embodiments, M is

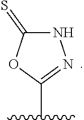 [SMILES: *c1n[nH]c(=S)o1]

In some embodiments, M is

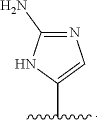 [SMILES: *c1cnc(N)[nH]1]

In some embodiments, M is

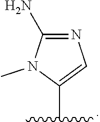 [SMILES: *c1cnc(N)n1C]

In some embodiments, M is

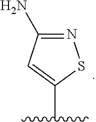 [SMILES: *c1cc(N)ns1]

In some embodiments, M is

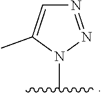 [SMILES: *n1nncc1C]

In some embodiments, M is

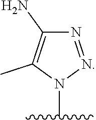 [SMILES: *n1nnc(N)c1C]

In some embodiments, the compound is a compound of Formula (C1-a), Formula (C1-b), Formula (C1-c), Formula (C1-d), Formula (C1-e), Formula (C1-f), Formula (C1-g), Formula (C1-h), Formula (C1-i), Formula (C1-j), Formula (C1-k), Formula (C1-l), Formula (C1-m), or Formula (C1-n):

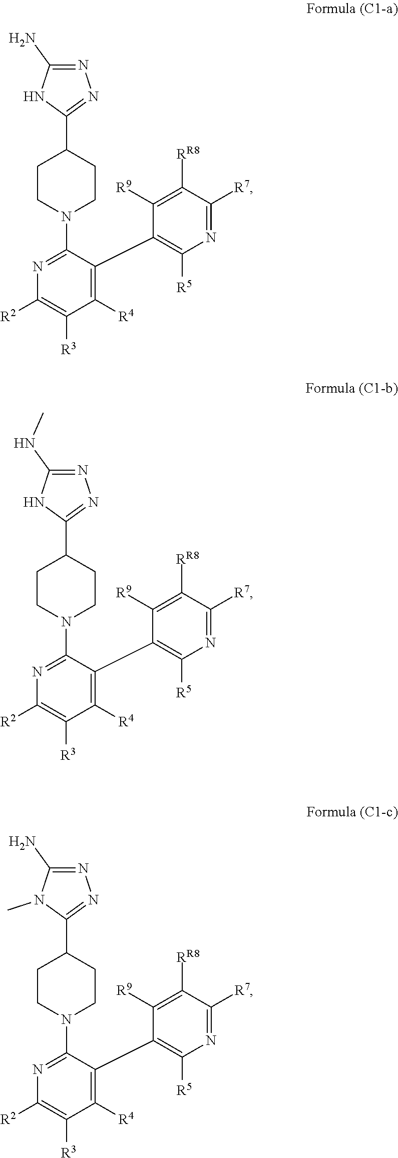

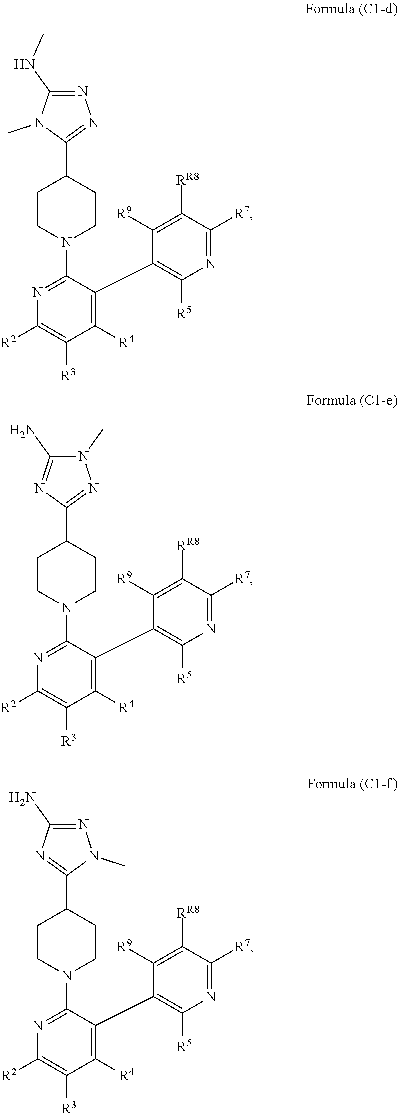

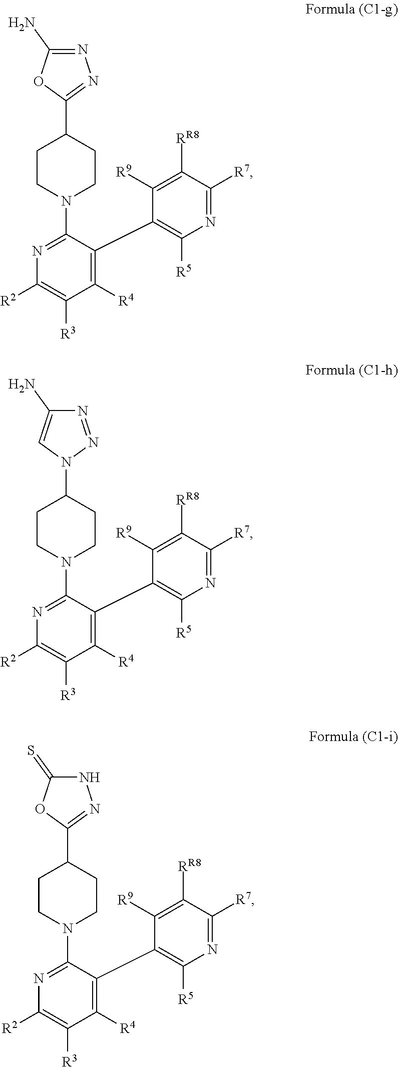

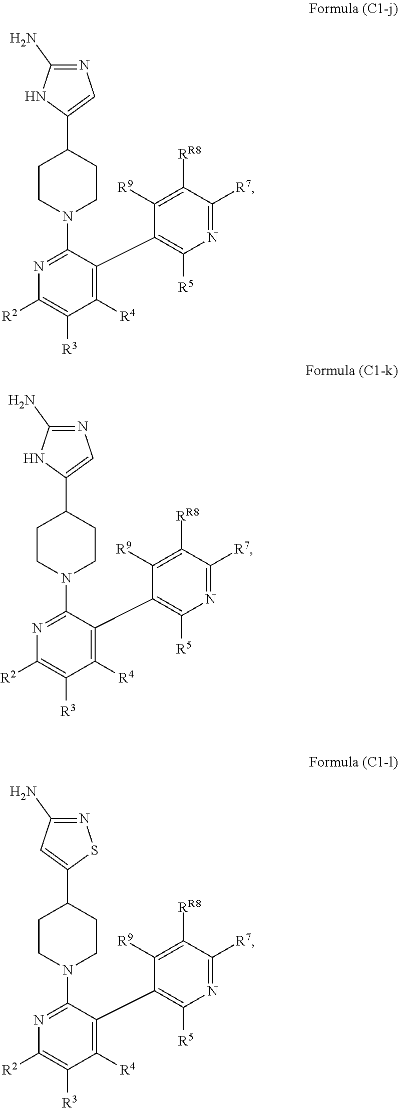

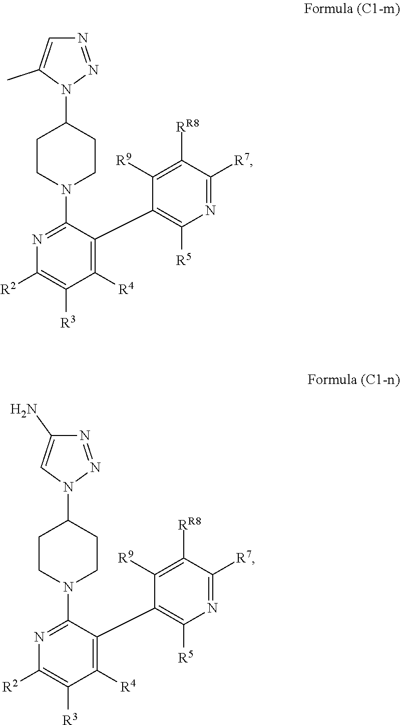

or a pharmaceutically acceptable salt, or solvate thereof.

In some embodiments, the compound is a compound of Formula (C1-a) or Formula (C1-c), or a pharmaceutically acceptable salt, or solvate thereof.

In some embodiments, the compound is a compound of Formula (C1-a), or a pharmaceutically acceptable salt, or solvate thereof. In some embodiments, the compound is a compound of Formula (C1-b), or a pharmaceutically acceptable salt, or solvate thereof. In some embodiments, the compound is a compound of Formula (C1-c), or a pharmaceutically acceptable salt, or solvate thereof. In some embodiments, the compound is a compound of Formula (C1-d), or a pharmaceutically acceptable salt, or solvate thereof. In some embodiments, the compound is a compound of Formula (C1-e), or a pharmaceutically acceptable salt, or solvate thereof. In some embodiments, the compound is a compound of Formula (C1-f), or a pharmaceutically acceptable salt, or solvate thereof. In some embodiments, the compound is a compound of Formula (C1-g), or a pharmaceutically acceptable salt, or solvate thereof. In some embodiments, the compound is a compound of Formula (C1-h), or a pharmaceutically acceptable salt, or solvate thereof. In some embodiments, the compound is a compound of Formula (C1-i), or a pharmaceutically acceptable salt, or solvate thereof. In some embodiments, the compound is a compound of Formula (C1-j), or a pharmaceutically acceptable salt, or solvate thereof. In some embodiments, the compound is a compound of Formula (C1-k), or a pharmaceutically acceptable salt, or solvate thereof. In some embodiments, the compound is a compound of Formula (C1-l), or a pharmaceutically acceptable salt, or solvate thereof. In some embodiments, the compound is a compound of Formula (C1-m), or a pharmaceutically acceptable salt, or solvate thereof. In some embodiments, the compound is a compound of Formula (C1-n), or a pharmaceutically acceptable salt, or solvate thereof.

In some embodiments, M is

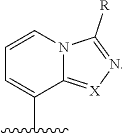

In some embodiments, M is

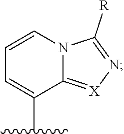

X is N or CH; and R is H, NH₂, or NHCH₃.

In some embodiments, X is N. In some embodiments, X is CH.

In some embodiments, R is H. In some embodiments, R is NH₂. In some embodiments, R is NHCH₃. In some embodiments, R is H or NH₂.

In some embodiments, X is N and R is NH₂. In some embodiments, X is N and R is H.

In some embodiments, X is CH and R is NH₂. In some embodiments, X is CH and R is H.

In some embodiments, M is

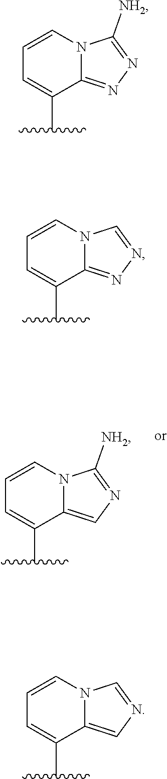 [SMILES: *c1cccn2c(N)nnc12 ; *c1cccn2cnnc12 ; *c1cccn2c(N)ncc12 ; "or" ; *c1cccn2cncc12]

In some embodiments, M is

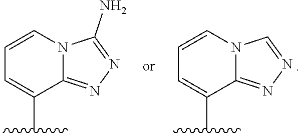 [SMILES: *c1cccn2c(N)nnc12 ; *c1cccn2cnnc12]

In some embodiments, M is

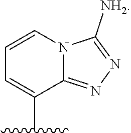 [SMILES: *c1cccn2c(N)nnc12]

In some embodiments, M is

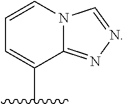 [SMILES: *c1cccn2cnnc12]

In some embodiments, M is

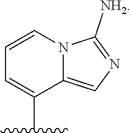 [SMILES: *c1cccn2c(N)ncc12]

In some embodiments, M is

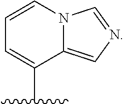 [SMILES: *c1cccn2cncc12]

In some embodiments, the compound is a compound of Formula (C1-o), Formula (C1-p), Formula (C1-q), or Formula (C1-r):

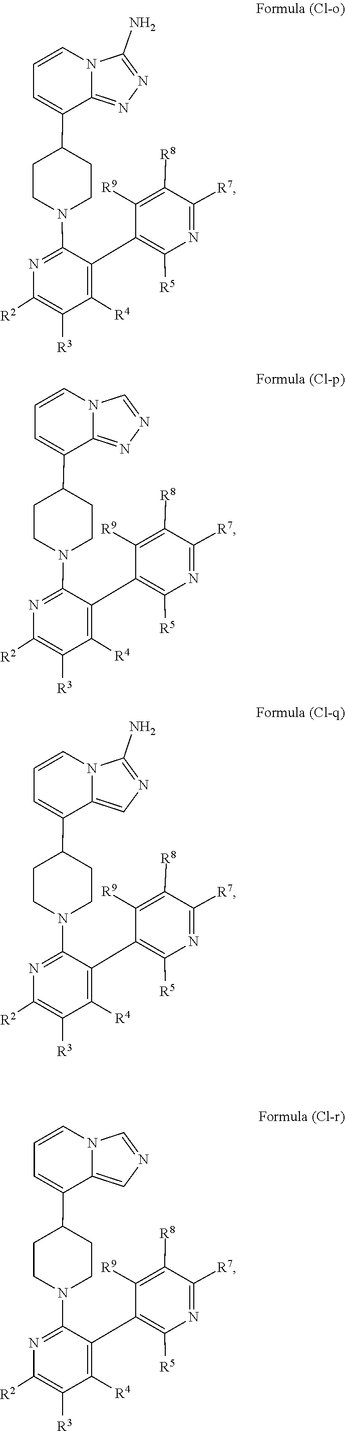

or a pharmaceutically acceptable salt, or solvate thereof.

In some embodiments, the compound is a compound of Formula (C1-o), or a pharmaceutically acceptable salt, or solvate thereof. In some embodiments, the compound is a compound of Formula (C1-p), or a pharmaceutically acceptable salt, or solvate thereof. In some embodiments, the compound is a compound of Formula (C1-q), or a pharmaceutically acceptable salt, or solvate thereof. In some embodiments, the compound is a compound of Formula (C1-r), or a pharmaceutically acceptable salt, or solvate thereof.

In some embodiments, the compound is a compound of Formula (C2-a), Formula (C2-b), Formula (C2-c), Formula (C2-d), Formula (C2-e), Formula (C2-f), Formula (C2-g), Formula (C2-h), Formula (C2-i), Formula (C2-j), Formula (C2-k), Formula (C2-l), Formula (C2-m), or Formula (C2-n):

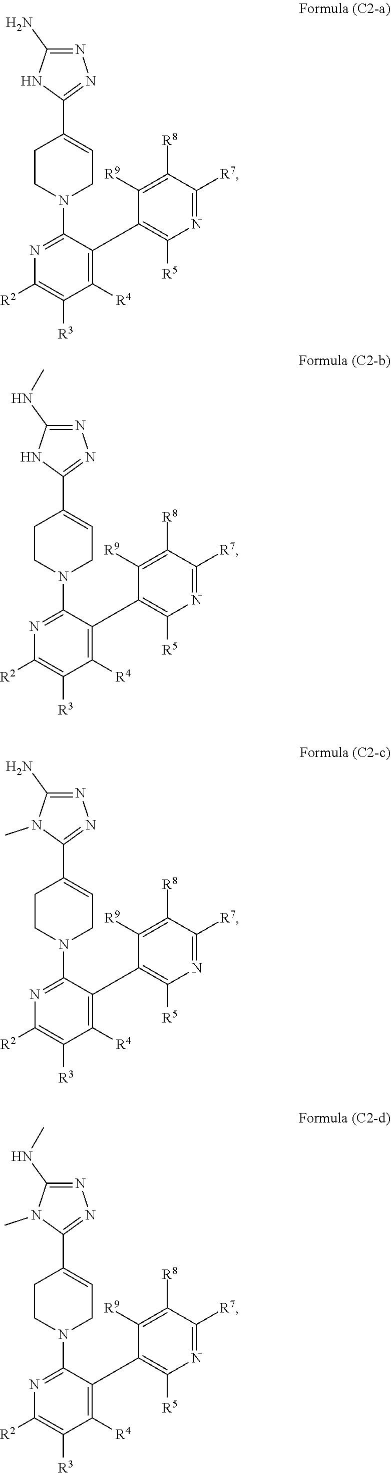

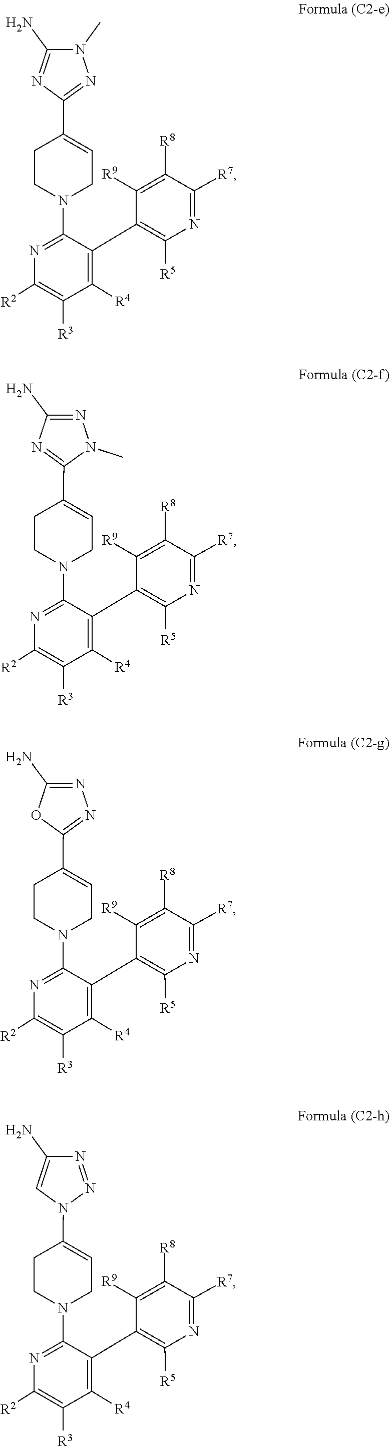

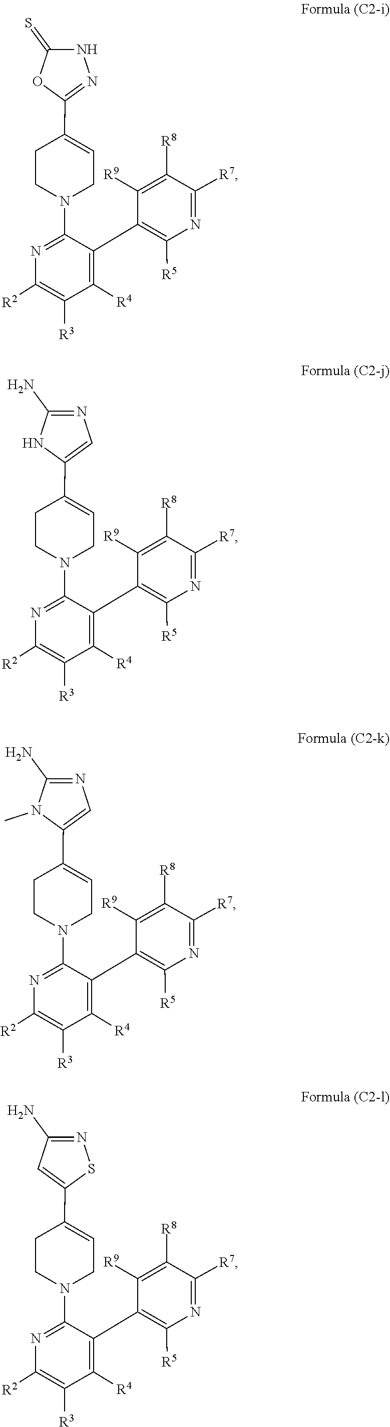

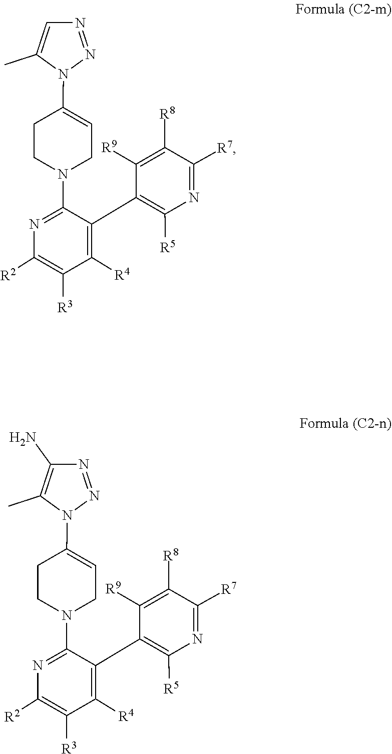

or a pharmaceutically acceptable salt, or solvate thereof.

In some embodiments, the compound is a compound of Formula (C2-a), or a pharmaceutically acceptable salt, or solvate thereof. In some embodiments, the compound is a compound of Formula (C2-b), or a pharmaceutically acceptable salt, or solvate thereof. In some embodiments, the compound is a compound of Formula (C2-c), or a pharmaceutically acceptable salt, or solvate thereof. In some embodiments, the compound is a compound of Formula (C2-d), or a pharmaceutically acceptable salt, or solvate thereof. In some embodiments, the compound is a compound of Formula (C2-e), or a pharmaceutically acceptable salt, or solvate thereof. In some embodiments, the compound is a compound of Formula (C2-f), or a pharmaceutically acceptable salt, or solvate thereof. In some embodiments, the compound is a compound of Formula (C2-g), or a pharmaceutically acceptable salt, or solvate thereof. In some embodiments, the compound is a compound of Formula (C2-h), or a pharmaceutically acceptable salt, or solvate thereof. In some embodiments, the compound is a compound of Formula (C2-i), or a pharmaceutically acceptable salt, or solvate thereof. In some embodiments, the compound is a compound of Formula (C2-j), or a pharmaceutically acceptable salt, or solvate thereof. In some embodiments, the compound is a compound of Formula (C2-k), or a pharmaceutically acceptable salt, or solvate thereof. In some embodiments, the compound is a compound of Formula (C2-l), or a pharmaceutically acceptable salt, or solvate thereof. In some embodiments, the compound is a compound of Formula (C2-m), or a pharmaceutically acceptable salt, or solvate thereof. In some embodiments, the compound is a compound of Formula (C2-n), or a pharmaceutically acceptable salt, or solvate thereof.

In some embodiments, the compound is a compound of Formula (C2-o), Formula (C2-p), Formula (C2-q), or Formula (C2-r):

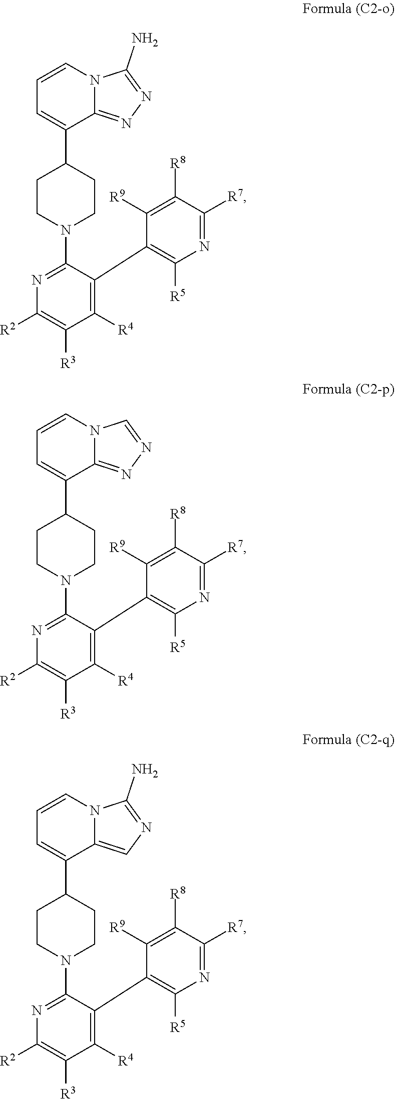

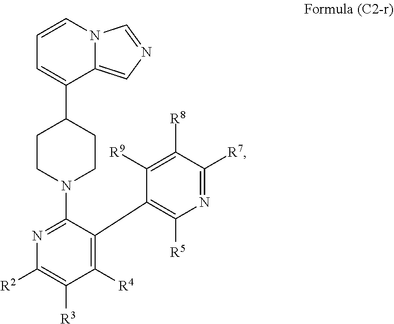

or a pharmaceutically acceptable salt, or solvate thereof.

In some embodiments, the compound is a compound of Formula (C2-o) or Formula (C2-p), or a pharmaceutically acceptable salt, or solvate thereof.

In some embodiments, the compound is a compound of Formula (C2-o), or a pharmaceutically acceptable salt, or solvate thereof. In some embodiments, the compound is a compound of Formula (C2-p), or a pharmaceutically acceptable salt, or solvate thereof. In some embodiments, the compound is a compound of Formula (C2-q), or a pharmaceutically acceptable salt, or solvate thereof. In some embodiments, the compound is a compound of Formula (C2-r), or a pharmaceutically acceptable salt, or solvate thereof.

In some embodiments, R¹ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R¹ is hydrogen, halogen, —CN, unsubstituted or substituted C₁-C₆ alkyl, or unsubstituted or substituted C₁-C₆ fluoroalkyl. In some embodiments, R¹ is hydrogen, halogen, —CN, methyl, ethyl, isopropyl, tert-butyl, difluoromethyl, or trifluoromethyl. In some embodiments, R¹ is hydrogen, halogen, or unsubstituted or substituted C₁-C₆ alkyl. In some embodiments, R¹ is hydrogen or halogen. In some embodiments, R² is unsubstituted or substituted C₁-C₆ alkyl. In some embodiments, R¹ is halogen. In some embodiments, R¹ is hydrogen.

In some embodiments, R² is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R² is hydrogen, halogen, —CN, unsubstituted or substituted C₁-C₆ alkyl, or unsubstituted or substituted C₁-C₆ fluoroalkyl. In some embodiments, R² is hydrogen, halogen, —CN, methyl, ethyl, isopropyl, tert-butyl, difluoromethyl, or trifluoromethyl. In some embodiments, R² is hydrogen, halogen, or unsubstituted or substituted C₁-C₆ alkyl. In some embodiments, R² is hydrogen or halogen. In some embodiments, R² is unsubstituted or substituted C₁-C₆ alkyl. In some embodiments, R² is halogen. In some embodiments, R² is hydrogen.

In some embodiments, R³ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R³ is hydrogen, halogen, —CN, unsubstituted or substituted C₁-C₆ alkyl, or unsubstituted or substituted C₁-C₆ fluoroalkyl. In some embodiments, R³ is hydrogen, halogen, —CN, methyl, ethyl, isopropyl, tert-butyl, difluoromethyl, or trifluoromethyl. In some embodiments, R³ is hydrogen, halogen, or unsubstituted or substituted C₁-C₆ alkyl. In some embodiments, R³ is hydrogen or halogen. In some embodiments, R³ is unsubstituted or substituted C₁-C₆ alkyl. In some embodiments, R³ is halogen. In some embodiments, R³ is hydrogen.

In some embodiments, R⁴ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁴ is hydrogen, halogen, —CN, unsubstituted or substituted C₁-C₆ alkyl, or unsubstituted or substituted C₁-C₆ fluoroalkyl. In some embodiments, R⁴ is hydrogen, halogen, —CN, methyl, ethyl, isopropyl, tert-butyl, difluoromethyl, or trifluoromethyl. In some embodiments, R⁴ is hydrogen, halogen, or unsubstituted or substituted C₁-C₆ alkyl. In some embodiments, R⁴ is hydrogen or halogen. In some embodiments, R⁴ is unsubstituted or substituted C₁-C₆ alkyl. In some embodiments, R⁴ is halogen. In some embodiments, R⁴ is hydrogen.

In some embodiments, R⁵ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁵ is hydrogen, halogen, —CN, unsubstituted or substituted C₁-C₆ alkyl, or unsubstituted or substituted C₁-C₆ fluoroalkyl. In some embodiments, R⁵ is hydrogen, halogen, —CN, methyl, ethyl, isopropyl, tert-butyl, difluoromethyl, or trifluoromethyl. In some embodiments, R⁵ is hydrogen, halogen, or unsubstituted or substituted C₁-C₆ alkyl. In some embodiments, R⁵ is hydrogen or halogen. In some embodiments, R⁵ is unsubstituted or substituted C₁-C₆ alkyl. In some embodiments, R⁵ is halogen. In some embodiments, R⁵ is hydrogen.

In some embodiments, R⁶ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁶ is hydrogen, halogen, —CN, unsubstituted or substituted C₁-C₆ alkyl, or unsubstituted or substituted C₁-C₆ fluoroalkyl. In some embodiments, R⁶ is hydrogen, halogen, —CN, methyl, ethyl, isopropyl, tert-butyl, difluoromethyl, or trifluoromethyl. In some embodiments, R⁶ is hydrogen, halogen, or unsubstituted or substituted C₁-C₆ alkyl. In some embodiments, R⁶ is hydrogen or halogen. In some embodiments, R⁵ is unsubstituted or substituted C₁-C₆ alkyl. In some embodiments, R⁶ is halogen. In some embodiments, R⁶ is hydrogen.

In some embodiments, R⁷ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁷ is hydrogen, halogen, —CN, unsubstituted or substituted C₁-C₆ alkyl, or unsubstituted or substituted C₁-C₆ fluoroalkyl. In some embodiments, R⁷ is hydrogen, halogen, —CN, methyl, ethyl, isopropyl, tert-butyl, difluoromethyl, or trifluoromethyl. In some embodiments, R⁷ is hydrogen, halogen, or unsubstituted or substituted C₁-C₆ alkyl. In some embodiments, R⁷ is hydrogen or halogen. In some embodiments, R⁷ is unsubstituted or substituted C₁-C₆ alkyl. In some embodiments, R⁷ is halogen. In some embodiments, R⁷ is hydrogen.

In some embodiments, R⁸ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁸ is hydrogen, halogen, —CN, unsubstituted or substituted C₁-C₆ alkyl, or unsubstituted or substituted C₁-C₆ fluoroalkyl. In some embodiments, R⁸ is hydrogen, halogen, —CN, methyl, ethyl, isopropyl, tert-butyl, difluoromethyl, or trifluoromethyl. In some embodiments, R⁸ is hydrogen, halogen, or unsubstituted or substituted C₁-C₆ alkyl. In some embodiments, R⁸ is hydrogen or halogen. In some embodiments, R⁸ is unsubstituted or substituted C₁-C₆ alkyl. In some embodiments, R⁸ is halogen. In some embodiments, R⁸ is hydrogen.

In some embodiments, R⁹ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁹ is hydrogen, halogen, —CN, unsubstituted or substituted C₁-C₆ alkyl, or unsubstituted or substituted C₁-C₆ fluoroalkyl. In some embodiments, R⁹ is hydrogen, halogen, —CN, methyl, ethyl, isopropyl, tert-butyl, difluoromethyl, or trifluoromethyl. In some embodiments, R⁹ is hydrogen, halogen, or unsubstituted or substituted C₁-C₆ alkyl. In some embodiments, R⁹ is hydrogen or halogen. In some embodiments, R⁹ is unsubstituted or substituted C₁-C₆ alkyl. In some embodiments, R⁹ is halogen. In some embodiments, R⁹ is hydrogen.

In some embodiments, R¹, R², R³, R⁴, R⁵, R⁶, R⁷, R⁸, and R⁹ are each independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R¹, R², R³, R⁴, R⁵, R⁶, R⁷, R⁸, and R⁹ are each independently hydrogen, halogen, —CN, unsubstituted or substituted C₁-C₆ alkyl, or unsubstituted or substituted C₁-C₆ fluoroalkyl. In some embodiments, R¹, R², R³, R⁴, R⁵, R⁶, R⁷, R⁸, and R⁹ are each independently hydrogen, halogen, —CN, methyl, ethyl, isopropyl, tert-butyl, difluoromethyl, or trifluoromethyl.

In some embodiments, R¹, R², R³, R⁴, R⁵, R⁶, R⁷, R⁸, and R⁹ are each independently hydrogen or halogen. In some embodiments, R¹, R², R³, R⁴, R⁵, R⁶, R⁷, R⁸, and R⁹ are each independently hydrogen or halogen. In some embodiments, R¹, R², R³, R⁴, R⁵, R⁶, R⁷, R⁸, and R⁹ are each independently hydrogen. In some embodiments, R¹, R², R³, R⁴, R⁵, R⁶, R⁷, R⁸, and R⁹ are each independently hydrogen and R⁷ is hydrogen, halogen, —CN, unsubstituted or substituted C₁-C₆ alkyl, or unsubstituted or substituted C₁-C₆ fluoroalkyl.

In some embodiments, R², R³, R⁴, R⁵, R⁷, R⁸, and R⁹ are each independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R¹, R², R³, R⁴, R⁵, R⁶, R⁷, R⁸, and R⁹ are each independently hydrogen, halogen, —CN, unsubstituted or substituted C₁-C₆ alkyl, or unsubstituted or substituted C₁-C₆ fluoroalkyl. In some embodiments, R², R³, R⁴, R⁵, R⁷, R⁸, and R⁹ are each independently hydrogen, —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃.

In some embodiments, R², R³, R⁴, R⁵, R⁷, R⁸, and R⁹ are each independently hydrogen, halogen, or C₁-C₆ alkyl. In some embodiments, R², R³, R⁴, R⁵, R⁷, R⁸, and R⁹ are each independently hydrogen, halogen, or C₁-C₄ alkyl. In some embodiments, R², R³, R⁴, R⁵, R⁷, R⁸, and R⁹ are each independently hydrogen, —F, —Cl, —CH₃, —CH₂CH₃, —CH(CH₃)₂, or —C(CH₃)₃. In some embodiments, R², R³, R⁴, R⁵, R⁷, R⁸, and R⁹ are each independently hydrogen, —F, or —CH₃.

In some embodiments, R³, R⁴, R⁵, R⁸, and R⁹ are each independently hydrogen or halogen. In some embodiments, R³, R⁴, R⁵, R⁸, and R⁹ are each hydrogen. In some embodiments, R³, R⁴, R⁵, R⁸, and R⁹ are each hydrogen; and R² and R⁷ are each independently R² and R⁷ are each independently hydrogen, halogen, —CN, —OR¹⁰, —N(R¹⁰)₂, C₁-C₆ alkyl, or C₁-C₆ fluoroalkyl.

In some embodiments, the compound is a compound of Formula (D):

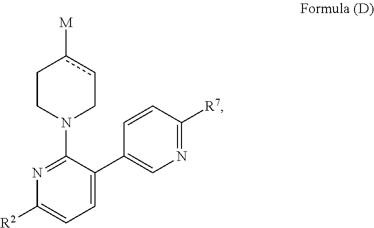

or a pharmaceutically acceptable salt, or solvate thereof. In some embodiments, R² and R⁷ are each independently hydrogen, halogen, —CN, —OR¹⁰, —N(R¹⁰)₂, C₁-C₆ alkyl, or C₁-C₆ fluoroalkyl.

In some embodiments, the compound is a compound of Formula (D1):

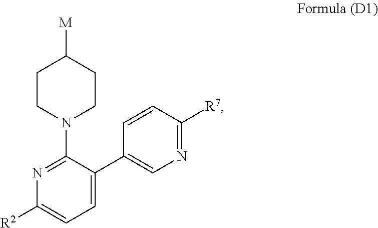

or a pharmaceutically acceptable salt, or solvate thereof. In some embodiments, R² and R⁷ are each independently hydrogen, halogen, —CN, —OR¹⁰, —N(R¹⁰)₂, C₁-C₆ alkyl, or C₁-C₆ fluoroalkyl.

In some embodiments, the compound is a compound of Formula (D2):

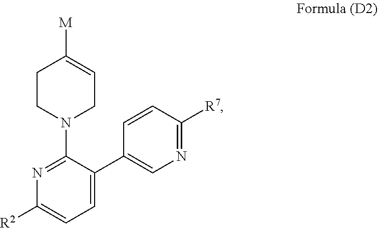

or a pharmaceutically acceptable salt, or solvate thereof. In some embodiments, R² and R⁷ are each independently hydrogen, halogen, —CN, —OR¹⁰, —N(R¹⁰)₂, C₁-C₆ alkyl, or C₁-C₆ fluoroalkyl.

In some embodiments, R² is hydrogen, halogen, —CN, —OR¹⁰, —N(R¹⁰)₂, C₁-C₆ alkyl, or C₁-C₆ fluoroalkyl. In some embodiments, R² is hydrogen, halogen, or C₁-C₆ alkyl. In some embodiments, R² is hydrogen, halogen, or C₁-C₄ alkyl. In some embodiments, R² is hydrogen, —F, —Cl, —CH₃, —CH₂CH₃, —CH(CH₃)₂, or —C(CH₃)₃. In some embodiments, R² is hydrogen, —F, or —CH₃. In some embodiments, R² is hydrogen. In some embodiments, R² is —F.

In some embodiment, s

In some embodiments, R⁷ is hydrogen, halogen, —CN, —OR¹⁰, —N(R¹⁰)₂, C₁-C₆ alkyl, or C₁-C₆ fluoroalkyl. In some embodiments, R⁷ is hydrogen, halogen, —CN, —OR¹⁰, or —N(R¹⁰)₂. In some embodiments, R⁷ is hydrogen, —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃. In some embodiments, R⁷ is hydrogen, —F, —Cl, —CN, —OCH₃, —OCH₂CH₃, —OCH₂CH₂OCH₃, —NH₂, —NHCH₃, or —N(CH₃)₂. In some embodiments, R⁷ is hydrogen, —F, —Cl, —CN, —OCH₃, —OCH₂CH₂OCH₃, or —NH₂. In some embodiments, R⁷ is hydrogen or halogen. In some embodiments, R⁷ is hydrogen, —F, or —Cl.

In some embodiments, R⁷ is halogen, —CN, —OR¹⁰, —N(R¹⁰)₂, C₁-C₆ alkyl, or C₁-C₆ fluoroalkyl. In some embodiments, R⁷ is halogen, —CN, —OR¹⁰, or —N(R¹⁰)₂. In some embodiments, R⁷ is —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃. In some embodiments, R⁷ is —F, —Cl, —CN, —OCH₃, —OCH₂CH₃, —OCH₂CH₂OCH₃, —NH₂, —NHCH₃, or —N(CH₃)₂. In some embodiments, R⁷ is —F, —Cl, —CN, —OCH₃, —OCH₂CH₂OCH₃, or —NH₂. In some embodiments, R⁷ is halogen. In some embodiments, R⁷ is —F or —Cl. In some embodiments, R⁷ is —F. In some embodiments, R⁷ is —Cl.

In some embodiments, R² is hydrogen, —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃; and R⁷ is hydrogen, —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃.

In some embodiments, R² is hydrogen, halogen, or C₁-C₄ alkyl; and R⁷ is halogen, —CN, —OR¹⁰, or —N(R¹⁰)₂. In some embodiments, R² is hydrogen, —F, —Cl, —CH₃, —CH₂CH₃, —CH(CH₃)₂, or —C(CH₃)₃; and R⁷ is —F, —Cl, —CN, —OCH₃, —OCH₂CH₃, —OCH₂CH₂OCH₃, —NH₂, —NHCH₃, or —N(CH₃)₂. In some embodiments, R² is hydrogen, —F, or —CH₃; R⁷ is hydrogen, —F, —Cl, —CN, —OCH₃, —OCH₂CH₂OCH₃, or —NH₂.

In one aspect, provided herein is a compound of Formula (I). In some embodiments, the compound of Formula (A) provided herein is a compound of Formula (I):

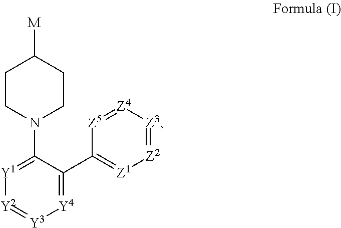

or a pharmaceutically acceptable salt, or solvate thereof, wherein:
M is L,

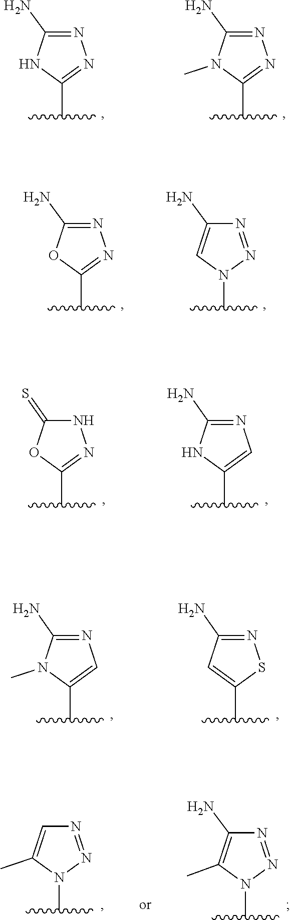

Y¹ is N or CR¹; Y² is N or CR²; Y³ is N or CR³; and Y⁴ is N or CR⁴;
Z¹ is N or CR⁵; Z² is N or CR⁶; Z³ is N or CR⁷; Z⁴ is N or CR⁸; and Z⁵ is N or CR⁹;
R¹, R², R³, R⁴, R⁵, R⁶, R⁷, R⁸, and R⁹ are each independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, —CN, —OR¹⁰, —CO₂R¹⁰, —C(═O)N(R¹⁰)₂, —N(R¹⁰)₂, —NR¹⁰C(═O)R¹¹, —SR¹⁰, —S(═O)R¹¹, —SO₂R¹¹, or —SO₂N(R¹⁰)₂;
each R¹⁰ is independently selected from hydrogen, unsubstituted or substituted C₁-C₆ alkyl, unsubstituted or substituted C₁-C₆ heteroalkyl, unsubstituted or substituted C₃-C₆ cycloalkyl, 4- to 6-membered heterocycloalkyl;
or two R¹⁰ on the same N atom are taken together with the N atom to which they are attached to form an unsubstituted or substituted N-containing 4- to 6-membered heterocycloalkyl; and
each R¹¹ is independently selected from unsubstituted or substituted C₁-C₆ alkyl, unsubstituted or substituted C₁-C₆ heteroalkyl, unsubstituted or substituted C₃-C₆ cycloalkyl, 4- to 6-membered heterocycloalkyl;
wherein each substituted alkyl, substituted alkenyl, substituted alkynyl, substituted fluoroalkyl, substituted heteroalkyl, substituted cycloalkyl, and substituted heterocycloalkyl is substituted with one or more R^{s} groups independently selected from the group consisting of halogen, C₁-C₆ alkyl, monocyclic carbocycle, monocyclic heterocycle, —CN, —OR¹², —CO₂R¹², —C(═O)N(R¹²)₂, —N(R¹²)₂, —NR¹²C(═O)R¹³, —SR¹², —S(═O)R¹³, —SO₂R¹³, or —SO₂N(R¹³)₂;
   each R¹² is independently selected from hydrogen, C₁-C₆ alkyl, C₁-C₆ heteroalkyl, C₃-C₆ cycloalkyl, 4- to 6-membered heterocycloalkyl, phenyl, 5-membered heteroaryl and 6-membered heteroaryl; or two R² groups are taken together with the N atom to which they are attached to form a N-containing 4- to 6-membered heterocycloalkyl;
   each R¹³ is independently selected from C₁-C₆ alkyl, C₁-C₆ heteroalkyl, C₃-C₆ cycloalkyl, 4- to 6-membered heterocycloalkyl, phenyl, 5-membered heteroaryl and 6-membered heteroaryl;
wherein 0, 1, or 2 of Y¹, Y², Y³, and Y⁴ are N; and
wherein 0, 1, or 2 of Z¹, Z², Z³, Z⁴, and Z⁵ are N.

In some embodiments, 0 or 1 of Y¹, Y², Y³, and Y⁴ is N. In some embodiments, 0 of Y¹, Y², Y³, and Y⁴ is N. In some embodiments, 1 of Y¹, Y², Y³, and Y⁴ is N.

In some embodiments, Y¹ is N or CR¹. In some embodiments, Y² is N or CR². In some embodiments, Y³ is N or CR³. In some embodiments, Y⁴ is N or CR⁴. In some embodiments, Y¹ is N. In some embodiments, Y¹ is CR¹. In some embodiments, Y² is N. In some embodiments, Y² is CR². In some embodiments, Y³ is N. In some embodiments, Y³ is CR³. In some embodiments, Y⁴ is N. In some embodiments, Y⁴ is CR⁴.

In some embodiments, Y¹ is CR¹; Y² is CR²; Y³ is CR³; and Y⁴ is CR⁴. In some embodiments, Y¹ is N; Y² is CR²; Y³ is CR³; and Y⁴ is CR⁴. In some embodiments, Y¹ is CR¹; Y² is N; Y³ is CR³; and Y⁴ is CR⁴. In some embodiments, Y¹ is CR¹; Y² is CR²; Y³ is N; and Y⁴ is CR⁴. In some embodiments, Y¹ is CR¹; Y² is CR²; Y³ is CR³; and Y⁴ is N.

In some embodiments, 0 or 1 of Z¹, Z², Z³, Z⁴, and Z⁵ is N. In some embodiments, 0 of Z¹, Z², Z³, Z⁴, and Z⁵ is N. In some embodiments, 1 of Z¹, Z², Z³, Z⁴, and Z⁵ is N.

In some embodiments, Z¹ is N or CR⁵. In some embodiments, Z² is N or CR⁶. In some embodiments, Z³ is N or CR⁷. In some embodiments, Z⁴ is N or CR⁸. In some embodiments, Z⁵ is N or CR⁹. In some embodiments, Z² is N. In some embodiments, Z² is CR⁶.

In some embodiments, Z¹ is CR⁵; Z² is CR⁶; Z³ is CR⁷; Z⁴ is CR⁸; and Z⁵ is CR⁹. In some embodiments, Z¹ is N; Z² is CR⁶; Z³ is CR⁷; Z⁴ is CR⁸; and Z⁵ is CR⁹. In some embodiments, Z¹ is CR⁵; Z² is N; Z³ is CR⁷; Z⁴ is CR⁸; and Z⁵ is CR⁹. In some embodiments, Z¹ is CR⁵; Z² is CR⁶; Z³ is N; Z⁴ is CR⁸; and Z⁵ is CR⁹. In some embodiments, Z¹ is CR⁵; Z² is CR⁶; Z³ is CR⁷; Z⁴ is N; and Z⁵ is CR⁹. In some embodiments, Z¹ is CR⁵; Z² is CR⁶; Z³ is CR⁷; Z⁴ is CR⁸; and Z⁵ is N.

In some embodiments, 0 or 1 of Y¹, Y², Y³, and Y⁴ is N; and 0 or 1 of Z¹, Z², Z³, Z⁴, and Z⁵ is N. In some embodiments, 0 of Y¹, Y², Y³, and Y⁴ is N; and 0 of Z¹, Z², Z³, Z⁴, and Z⁵ is N. In some embodiments, 0 of Y¹, Y², Y³, and Y⁴ is N; and 1 of Z¹, Z², Z³, Z⁴, and Z⁵ is N. In some embodiments, 1 of Y¹, Y², Y³, and Y⁴ is N; and 0 of Z¹, Z², Z³, Z⁴, and Z⁵ is N. In some embodiments, 1 of Y¹, Y², Y³, and Y⁴ is N; and 1 of Z¹, Z², Z³, Z⁴, and Z⁵ is N. In some embodiments, 1 of Y¹ and Y³ is N; and 1 of Z² and Z⁴ is N. In some embodiments, Y¹ is N; and 1 of Z² and Z⁴ is N. In some embodiments, 1 of Y¹ and Y³ is N; and Z² is N.

In some embodiments, the compound is a compound of Formula (II):

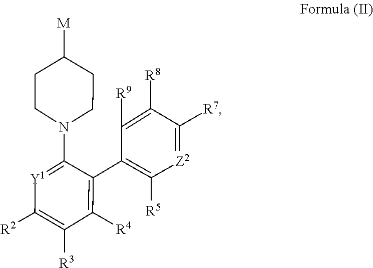

or a pharmaceutically acceptable salt, or solvate thereof.

In some embodiments, Y¹ is N; and Z² is N or CR⁶. In some embodiments, Y¹ is N; and Z² is CR⁶. In some embodiments, Y¹ is N; and Z² is N. In some embodiments, Y¹ is CR¹; and Z² is N or CR⁶. In some embodiments, Y¹ is CR¹; and Z² is CR⁶. In some embodiments, Y¹ is CR¹; and Z² is N.

In some embodiments, the compound is a compound of Formula (III):

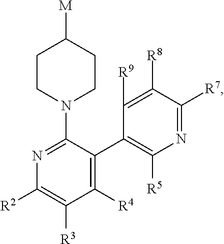

or a pharmaceutically acceptable salt, or solvate thereof.

In some embodiments, M is

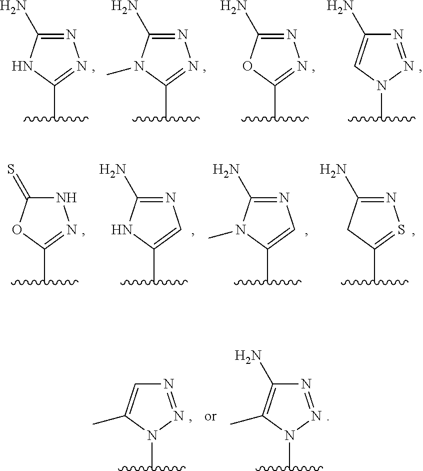

In some embodiments, M is

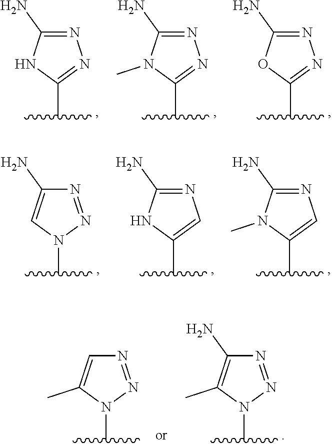

In some embodiments, M is

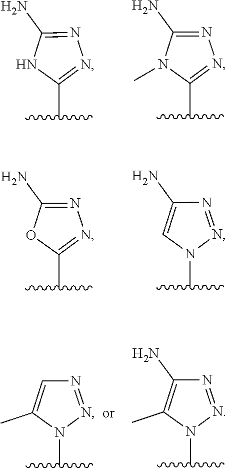

In some embodiments, M is

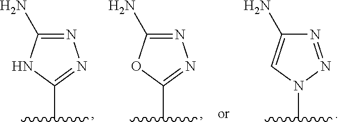 [SMILES: *c1nnc(N)[nH]1 ; *c1nnc(N)o1 ; *n1cc(N)nn1]

In some embodiments, M is

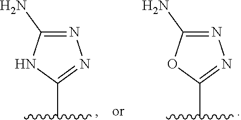 [SMILES: *c1nnc(N)[nH]1 ; *c1nnc(N)o1]

In some embodiments, M is

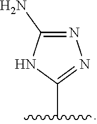 [SMILES: *c1nnc(N)[nH]1]

In some embodiments, M is

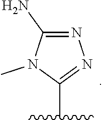 [SMILES: *c1nnc(N)n1C]

In some embodiments, M is

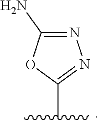 [SMILES: *c1nnc(N)o1]

In some embodiments, M is

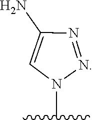 [SMILES: *n1cc(N)nn1]

In some embodiments, M is

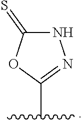 [SMILES: *c1n[nH]c(=S)o1]

In some embodiments, M is

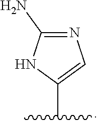 [SMILES: *c1cnc(N)[nH]1]

In some embodiments, M is

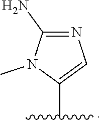 [SMILES: *c1cnc(N)n1C]

In some embodiments, M is

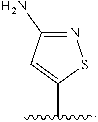 [SMILES: *c1cc(N)ns1]

In some embodiments, M is

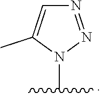 [SMILES: *n1nncc1C]

In some embodiments, M is

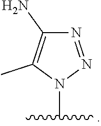 [SMILES: *n1nnc(N)c1C]

In some embodiments, R¹ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R¹ is hydrogen, halogen, —CN, unsubstituted or substituted C₁-C₆ alkyl, or unsubstituted or substituted C₁-C₆ fluoroalkyl. In some embodiments, R¹ is hydrogen, halogen, —CN, methyl, ethyl, isopropyl, tert-butyl, difluoromethyl, or trifluoromethyl. In some embodiments, R¹ is hydrogen, halogen, or unsubstituted or substituted C₁-C₆ alkyl. In some embodiments, R¹ is hydrogen or halogen. In some embodiments, R² is unsubstituted or substituted C₁-C₆ alkyl. In some embodiments, R¹ is halogen. In some embodiments, R¹ is hydrogen.

In some embodiments, R² is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R² is hydrogen, halogen, —CN, unsubstituted or substituted C₁-C₆ alkyl, or unsubstituted or substituted C₁-C₆ fluoroalkyl. In some embodiments, R² is hydrogen, halogen, —CN, methyl, ethyl, isopropyl, tert-butyl, difluoromethyl, or trifluoromethyl. In some embodiments, R² is hydrogen, halogen, or unsubstituted or substituted C₁-C₆ alkyl. In some embodiments, R² is hydrogen or halogen. In some embodiments, R² is unsubstituted or substituted C₁-C₆ alkyl. In some embodiments, R² is halogen. In some embodiments, R² is hydrogen.

In some embodiments, R³ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R³ is hydrogen, halogen, —CN, unsubstituted or substituted C₁-C₆ alkyl, or unsubstituted or substituted C₁-C₆ fluoroalkyl. In some embodiments, R³ is hydrogen, halogen, —CN, methyl, ethyl, isopropyl, tert-butyl, difluoromethyl, or trifluoromethyl. In some embodiments, R³ is hydrogen, halogen, or unsubstituted or substituted C₁-C₆ alkyl. In some embodiments, R³ is hydrogen or halogen. In some embodiments, R³ is unsubstituted or substituted C₁-C₆ alkyl. In some embodiments, R³ is halogen. In some embodiments, R³ is hydrogen.

In some embodiments, R⁴ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁴ is hydrogen, halogen, —CN, unsubstituted or substituted C₁-C₆ alkyl, or unsubstituted or substituted C₁-C₆ fluoroalkyl. In some embodiments, R⁴ is hydrogen, halogen, —CN, methyl, ethyl, isopropyl, tert-butyl, difluoromethyl, or trifluoromethyl. In some embodiments, R⁴ is hydrogen, halogen, or unsubstituted or substituted C₁-C₆ alkyl. In some embodiments, R⁴ is hydrogen or halogen. In some embodiments, R⁴ is unsubstituted or substituted C₁-C₆ alkyl. In some embodiments, R⁴ is halogen. In some embodiments, R⁴ is hydrogen.

In some embodiments, R⁵ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁵ is hydrogen, halogen, —CN, unsubstituted or substituted C₁-C₆ alkyl, or unsubstituted or substituted C₁-C₆ fluoroalkyl. In some embodiments, R⁵ is hydrogen, halogen, —CN, methyl, ethyl, isopropyl, tert-butyl, difluoromethyl, or trifluoromethyl. In some embodiments, R⁵ is hydrogen, halogen, or unsubstituted or substituted C₁-C₆ alkyl. In some embodiments, R⁵ is hydrogen or halogen. In some embodiments, R⁵ is unsubstituted or substituted C₁-C₆ alkyl. In some embodiments, R⁵ is halogen. In some embodiments, R⁵ is hydrogen.

In some embodiments, R⁶ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁶ is hydrogen, halogen, —CN, unsubstituted or substituted C₁-C₆ alkyl, or unsubstituted or substituted C₁-C₆ fluoroalkyl. In some embodiments, R⁶ is hydrogen, halogen, —CN, methyl, ethyl, isopropyl, tert-butyl, difluoromethyl, or trifluoromethyl. In some embodiments, R⁶ is hydrogen, halogen, or unsubstituted or substituted C₁-C₆ alkyl. In some embodiments, R⁶ is hydrogen or halogen. In some embodiments, R⁵ is unsubstituted or substituted C₁-C₆ alkyl. In some embodiments, R⁶ is halogen. In some embodiments, R⁶ is hydrogen.

In some embodiments, R⁷ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁷ is hydrogen, halogen, —CN, unsubstituted or substituted C₁-C₆ alkyl, or unsubstituted or substituted C₁-C₆ fluoroalkyl. In some embodiments, R⁷ is hydrogen, halogen, —CN, methyl, ethyl, isopropyl, tert-butyl, difluoromethyl, or trifluoromethyl. In some embodiments, R⁷ is hydrogen, halogen, or unsubstituted or substituted C₁-C₆ alkyl. In some embodiments, R⁷ is hydrogen or halogen. In some embodiments, R⁷ is unsubstituted or substituted C₁-C₆ alkyl. In some embodiments, R⁷ is halogen. In some embodiments, R⁷ is hydrogen.

In some embodiments, R⁸ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁸ is hydrogen, halogen, —CN, unsubstituted or substituted C₁-C₆ alkyl, or unsubstituted or substituted C₁-C₆ fluoroalkyl. In some embodiments, R⁸ is hydrogen, halogen, —CN, methyl, ethyl, isopropyl, tert-butyl, difluoromethyl, or trifluoromethyl. In some embodiments, R⁸ is hydrogen, halogen, or unsubstituted or substituted C₁-C₆ alkyl. In some embodiments, R⁸ is hydrogen or halogen. In some embodiments, R⁸ is unsubstituted or substituted C₁-C₆ alkyl. In some embodiments, R⁸ is halogen. In some embodiments, R⁸ is hydrogen.

In some embodiments, R⁹ is hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R⁹ is hydrogen, halogen, —CN, unsubstituted or substituted C₁-C₆ alkyl, or unsubstituted or substituted C₁-C₆ fluoroalkyl. In some embodiments, R⁹ is hydrogen, halogen, —CN, methyl, ethyl, isopropyl, tert-butyl, difluoromethyl, or trifluoromethyl. In some embodiments, R⁹ is hydrogen, halogen, or unsubstituted or substituted C₁-C₆ alkyl. In some embodiments, R⁹ is hydrogen or halogen. In some embodiments, R⁹ is unsubstituted or substituted C₁-C₆ alkyl. In some embodiments, R⁹ is halogen. In some embodiments, R⁹ is hydrogen.

In some embodiments, R¹, R², R³, R⁴, R⁵, R⁶, R⁷, R⁸, and R⁹ are each independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R¹, R², R³, R⁴, R⁵, R⁶, R⁷, R⁸, and R⁹ are each independently hydrogen, halogen, —CN, unsubstituted or substituted C₁-C₆ alkyl, or unsubstituted or substituted C₁-C₆ fluoroalkyl. In some embodiments, R¹, R², R³, R⁴, R⁵, R⁶, R⁷, R⁸, and R⁹ are each independently hydrogen, halogen, —CN, methyl, ethyl, isopropyl, tert-butyl, difluoromethyl, or trifluoromethyl.

In some embodiments, R¹, R², R³, R⁴, R⁵, R⁶, R⁷, R⁸, and R⁹ are each independently hydrogen or halogen. In some embodiments, R¹, R², R³, R⁴, R⁵, R⁶, R⁷, R⁸, and R⁹ are each independently hydrogen or halogen. In some embodiments, R¹, R², R³, R⁴, R⁵, R⁶, R⁷, R⁸, and R⁹ are each independently hydrogen. In some embodiments, R¹, R², R³, R⁴, R⁵, R⁶, R⁷, R⁸, and R⁹ are each independently hydrogen and R⁷ is hydrogen, halogen, —CN, unsubstituted or substituted C₁-C₆ alkyl, or unsubstituted or substituted C₁-C₆ fluoroalkyl.

In some embodiments, R², R³, R⁴, R⁵, R⁷, R⁸, and R⁹ are each independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, or —CN. In some embodiments, R¹, R², R³, R⁴, R⁵, R⁶, R⁷, R⁸, and R⁹ are each independently hydrogen, halogen, —CN, unsubstituted or substituted C₁-C₆ alkyl, or unsubstituted or substituted C₁-C₆ fluoroalkyl. In some embodiments, R², R³, R⁴, R⁵, R⁷, R⁸, and R⁹ are each independently hydrogen, halogen, —CN, methyl, ethyl, isopropyl, tert-butyl, difluoromethyl, or trifluoromethyl.

In some embodiments, R², R³, R⁴, R⁵, R⁷, R⁸, and R⁹ are each independently hydrogen or halogen. In some embodiments, R², R³, R⁴, R⁵, R⁸, and R⁹ are each independently hydrogen or halogen. In some embodiments, R², R³, R⁴, R⁵, R⁸, and R⁹ are each independently hydrogen. In some embodiments, R², R³, R⁴, R⁵, R⁸, and R⁹ are each independently hydrogen and R⁷ is hydrogen, halogen, —CN, unsubstituted or substituted C₁-C₆ alkyl, or unsubstituted or substituted C₁-C₆ fluoroalkyl.

In some embodiments, the compound is a compound of Formula (IV):

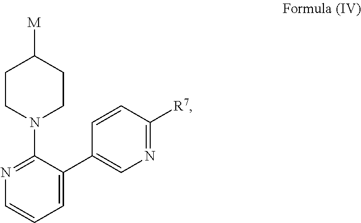

or a pharmaceutically acceptable salt, or solvate thereof; wherein: R⁷ is hydrogen, halogen, —CN, unsubstituted or substituted C₁-C₆ alkyl, or unsubstituted or substituted C₁-C₆ fluoroalkyl.

In some embodiments, R⁷ is hydrogen, halogen, —CN, unsubstituted or substituted C₁-C₆ alkyl. In some embodiments, R⁷ is hydrogen, halogen, —CN, methyl, ethyl, isopropyl, tert-butyl, difluoromethyl, or trifluoromethyl. In some embodiments, R⁷ is hydrogen or halogen. In some embodiments, R⁷ is hydrogen, fluorine, or chlorine. In some embodiments, R⁷ is hydrogen. In some embodiments, R⁷ is fluorine or chlorine. In some embodiments, R⁷ is fluorine. In some embodiments, R⁷ is chlorine.

In another aspect, provided herein is a compound of Formula (XI):

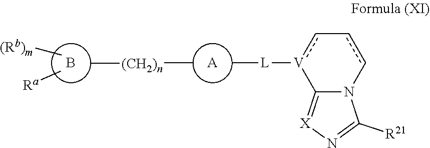

or a pharmaceutically acceptable salt, or solvate thereof, wherein:
R²¹ is H or NH₂;
X is N or CH;
V is N;
or V is C and [symbol] is a double bond;
or V is CH and [symbol] is a single bond;
L is absent, C₁-C₄ alkylene, C₂-C₄ alkenylene, or C₂-C₄ alkynylene;
Ring A is a tetrahydropyridine ring or a piperidine ring;
Ring B is phenyl, 5-membered heteroaryl, or 6-membered heteroaryl;
R^{a} is selected from hydrogen, halogen, unsubstituted or substituted C₁-C₆ alkyl, unsubstituted or substituted C₁-C₆ alkenyl, unsubstituted or substituted C₁-C₆ alkynyl, unsubstituted or substituted C₁-C₆ fluoroalkyl, unsubstituted or substituted C₁-C₆ heteroalkyl, unsubstituted or substituted carbocycle, unsubstituted or substituted heterocycle, —CN, —OR²², —CO₂R²², —C(═O)N(R²²)₂, —N(R²²)₂, —NR²²C(═O)R²³, —SR²², —S(═O)R²³, —SO₂R²³, and —SO₂N(R²²)₂;
each R^{b} is independently selected from hydrogen, halogen, unsubstituted or substituted C₁-C₆ alkyl, unsubstituted or substituted C₁-C₆ alkenyl, unsubstituted or substituted C₁-C₆ alkynyl, unsubstituted or substituted C₁-C₆ fluoroalkyl, unsubstituted or substituted C₁-C₆ heteroalkyl, unsubstituted or substituted carbocycle, unsubstituted or substituted heterocycle, —CN, —OR²², —CO₂R²², —C(═O)N(R²²)₂, —N(R²²)₂, —NR²²C(═O)R²³, —SR²², —S(═O)R²³, —SO₂R²³, and —SO₂N(R²²)₂;
each R²² is independently selected from hydrogen, unsubstituted or substituted C₁-C₆ alkyl, unsubstituted or substituted C₁-C₆ heteroalkyl, unsubstituted or substituted C₃-C₆ cycloalkyl, and 4- to 6-membered heterocycloalkyl;
or two R²² on the same N atom are taken together with the N atom to which they are attached to form an unsubstituted or substituted N-containing 4- to 6-membered heterocycloalkyl;
each R²³ is independently selected from unsubstituted or substituted C₁-C₆ alkyl, unsubstituted or substituted C₁-C₆ heteroalkyl, unsubstituted or substituted C₃-C₆ cycloalkyl, 4- to 6-membered heterocycloalkyl; and
wherein each substituted alkyl, substituted alkenyl, substituted alkynyl, substituted fluoroalkyl, substituted heteroalkyl, substituted carbocycle, substituted heterocycle, substituted cycloalkyl, and substituted heterocycloalkyl is substituted with one or more R^{s} groups independently selected from the group consisting of halogen, C₁-C₆ alkyl, monocyclic carbocycle, monocyclic heterocycle, —CN, —OR²⁴, —CO₂R²⁴, —C(═O)N(R²⁴)₂, —N(R²⁴)₂, —NR²⁴C(═O)R²⁵, —SR²⁴, —S(═O)R²⁵, —SO₂R²⁵, or —SO₂N(R²⁴)₂;
   each R²⁴ is independently selected from hydrogen, C₁-C₆ alkyl, C₁-C₆ heteroalkyl, C₃-C₆ cycloalkyl, 4- to 6-membered heterocycloalkyl, phenyl, 5-membered heteroaryl and 6-membered heteroaryl; or two R¹² groups are taken together with the N atom to which they are attached to form a N-containing 4- to 6-membered heterocycloalkyl;
   each R²⁵ is independently selected from C₁-C₆ alkyl, C₁-C₆ heteroalkyl, C₃-C₆ cycloalkyl, 4- to 6-membered heterocycloalkyl, phenyl, 5-membered heteroaryl and 6-membered heteroaryl;
m is 1, 2, 3, or 4;
n is 0 or 1; and
each [symbol] independently represents a single bond or a double bond; wherein when V is N, each [symbol] represents a single bond.

In some embodiments, Ring A is

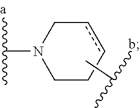

wherein [symbol] represents a single bond or a double bond; and

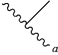

represents the attachment point to L; or

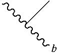

represents the attachment point to L. In some embodiments, [symbol] represents a single bond. In some embodiments, [symbol] represents a double bond. In some embodiments, Ring A is

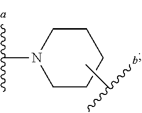

wherein

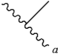

represents the attachment point to L; or

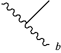

represents the attachment point to L. In some embodiments, Ring A is

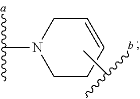

wherein

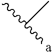

represents the attachment point to L; or

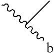

represents the attachment point to L. In some embodiments,

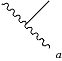

represents the attachment point to L. In some embodiments,

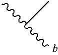

represents the attachment point to L.

In some embodiments, Ring A is

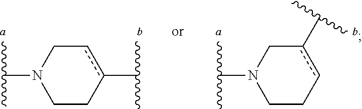

wherein [symbol] represents a single bond or a double bond; and

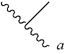

represents the attachment point to L; or

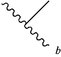

represents the attachment point to L. In some embodiments, [symbol] represents a single bond. In some embodiments, [symbol] represents a double bond. In some embodiments, Ring A is

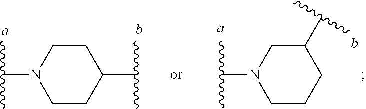 [SMILES: *C1CCN(*)CC1 ; *C1CCCN(*)C1]

wherein

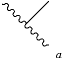

represents the attachment point to L; or

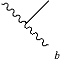

represents the attachment point to L. In some embodiments, Ring A is

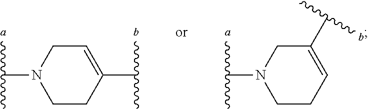 [SMILES: *C1=CCN(*)CC1 ; *C1=CCCN(*)C1]

wherein

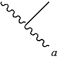

represents the attachment point to L; or

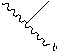

represents the attachment point to L. In some embodiments,

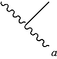

represents the attachment point to L. In some embodiments,

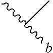

represents the attachment point to L.

In some embodiments, Ring A is

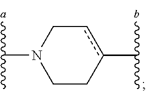

wherein [symbol] represents a single bond or a double bond; and

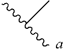

represents the attachment point to L; or

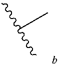

represents the attachment point to L. In some embodiments, [symbol] represents a single bond. In some embodiments, [symbol] represents a double bond. In some embodiments, Ring A is

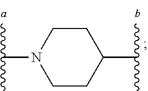 [SMILES: *C1CCN(*)CC1]

wherein

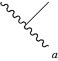

represents the attachment point to L; or

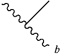

represents the attachment point to L. In some embodiments, Ring A is

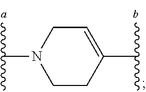 [SMILES: *C1=CCN(*)CC1]

wherein

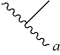

represents the attachment point to L; or

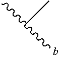

represents the attachment point to L. In some embodiments,

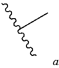

represents the attachment point to L. In some embodiments,

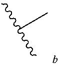

represents the attachment point to L.

In some embodiments, Ring A is

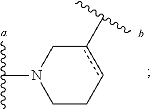

wherein [symbol] represents a single bond or a double bond; and

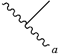

represents the attachment point to L; or

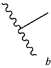

represents the attachment point to L. In some embodiments, [symbol] represents a single bond. In some embodiments, [symbol] represents a double bond. In some embodiments, Ring A is

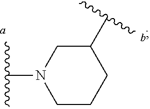 [SMILES: *C1CCCN(*)C1]

wherein

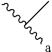

represents the attachment point to L; or

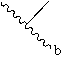

represents the attachment point to L. In some embodiments, Ring A is

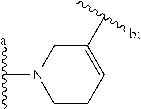

wherein

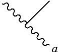

represents the attachment point to L; or

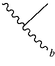

represents the attachment point to L. In some embodiments,

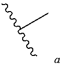

represents the attachment point to L. In some embodiments,

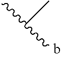

represents the attachment point to L.

In some embodiments, L is absent.

In some embodiments, L is C₁-C₄ alkylene, C₂-C₄ alkenylene, or C₂-C₄ alkynylene. In some embodiments, L is C₁-C₄ alkylene. In some embodiments, L is C₂-C₄ alkenylene. In some embodiments, L is C₂-C₄ alkynylene.

In some embodiments, L is absent or C₂-C₄ alkynylene. In some embodiments, L is absent or C₃ alkynylene.

In some embodiments, Ring A is

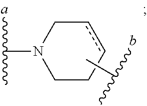

wherein [symbol] represents a single bond or a double bond;

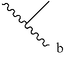

represents the attachment point to L; and L is absent.

In some embodiments, Ring A is

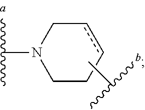

wherein [symbol] represents a single bond or a double bond;

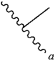

represents the attachment point to L; and L is C₂-C₄ alkynylene. In some embodiments, L is C₃ alkylene.

In some embodiments, the compound is a compound of Formula (XII):

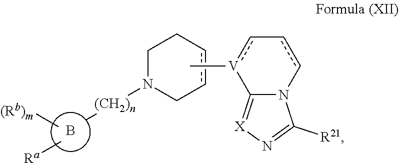

or a pharmaceutically acceptable salt, or solvate thereof, wherein each [symbol] independently represents a single bond or a double bond.

In some embodiments, the compound is a compound of Formula (XIIa) or Formula (XIIb):

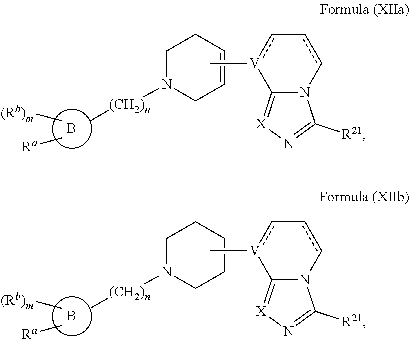

or a pharmaceutically acceptable salt, or solvate thereof, wherein each [symbol] independently represents a single bond or a double bond.

In some embodiments, the compound is a compound of Formula (XIIa). In some embodiments, the compound is a compound of Formula (XIIb).

In some embodiments the compound is a compound of Formula (XVII):

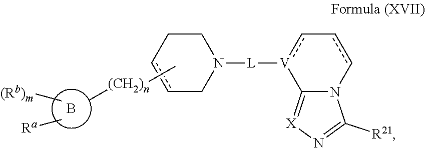

or a pharmaceutically acceptable salt, or solvate thereof, wherein each [symbol] independently represents a single bond or a double bond; and L is C₁-C₄ alkylene, C₂-C₄ alkenylene, or C₂-C₄ alkynylene. In some embodiments, L is C₂-C₄ alkynylene.

In some embodiments, the compound is a compound of Formula (XVIIa) or Formula (XVIIb):

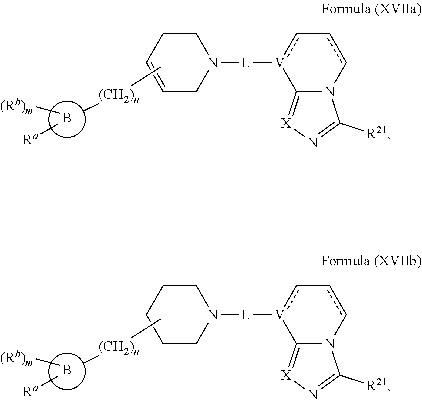

or a pharmaceutically acceptable salt, or solvate thereof, wherein each [symbol] independently represents a single bond or a double bond; and L is C₁-C₄ alkylene, C₂-C₄ alkenylene, or C₂-C₄ alkynylene. In some embodiments, L is C₂-C₄ alkynylene.

In some embodiments, the compound is a compound of Formula (XVIIa). In some embodiments, the compound is a compound of Formula (XVIIb).

In some embodiments, the compound is a compound of Formula (XVIIIa) or Formula (XVIIIb):

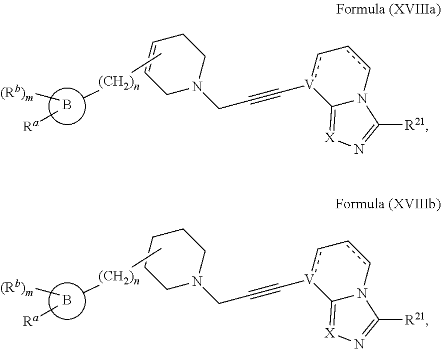

or a pharmaceutically acceptable salt, or solvate thereof, wherein each [symbol] independently represents a single bond or a double bond.

In some embodiments, the compound is a compound of Formula (XVIIIa). In some embodiments, the compound is a compound of Formula (XVIIIb).

In some embodiments, n is 1. In some embodiments, n is 0.

In some embodiments, the compound is a compound of Formula (XIII):

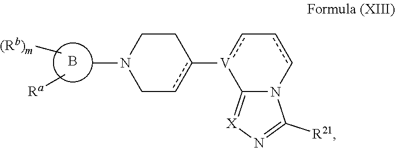

or a pharmaceutically acceptable salt, or solvate thereof, wherein each [symbol] independently represents a single bond or a double bond.

In some embodiments, the compound is a compound of Formula (XIIIa) or Formula (XIIIb):

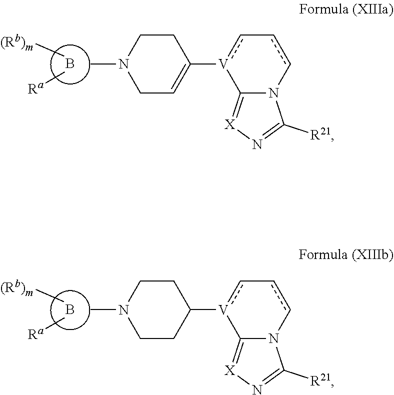

or a pharmaceutically acceptable salt, or solvate thereof, wherein each [symbol] independently represents a single bond or a double bond.

In some embodiments, the compound is a compound of Formula (XIIIa). In some embodiments, the compound is a compound of Formula (XIIIb).

In some embodiments, the compound is a compound of Formula (XIX):

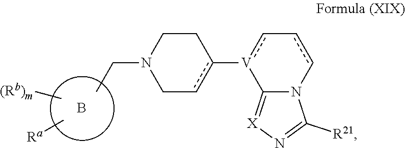

or a pharmaceutically acceptable salt, or solvate thereof, wherein each [symbol] independently represents a single bond or a double bond.

In some embodiments, the compound is a compound of Formula (XIXa) or Formula (XIXb):

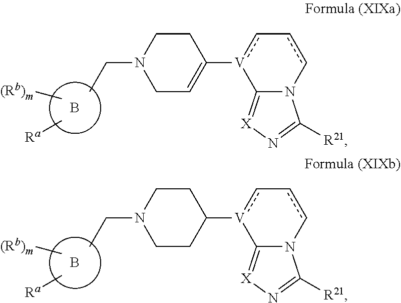

or a pharmaceutically acceptable salt, or solvate thereof, wherein each [symbol] independently represents a single bond or a double bond.

In some embodiments, the compound is a compound of Formula (XIXa). In some embodiments, the compound is a compound of Formula (XIXb).

In some embodiments, Ring B is phenyl, furanyl, thienyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, or triazinyl. In some embodiments, Ring B is phenyl, pyridinyl, pyrimidinyl, pyrazinyl, or pyridazinyl. In some embodiments, Ring B is phenyl or pyridinyl. In some embodiments, Ring B is phenyl. In some embodiments, Ring B is pyridinyl.

In some embodiments,

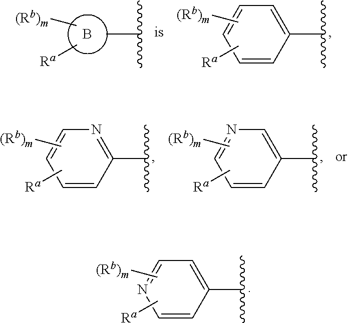

In some embodiments,

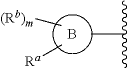

is

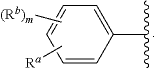

In some embodiments,

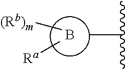

is

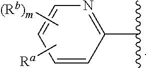

In some embodiments,

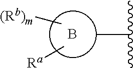

is

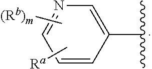

In some embodiments,

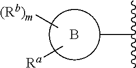

is

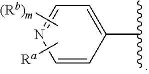

In some embodiments,

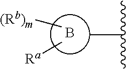

is

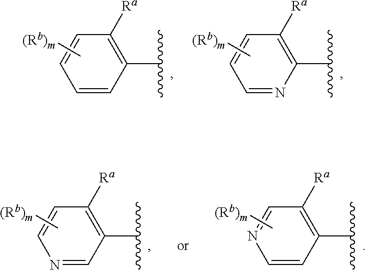

In some embodiments,

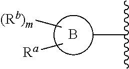

is

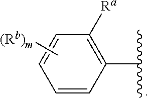

In some embodiments,

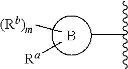

is

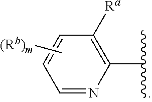

In some embodiments,

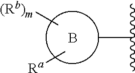

is

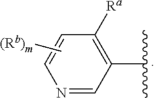

In some embodiments,

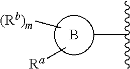

is

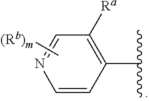

In some embodiments,

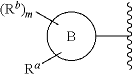

is

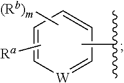

wherein W is N or CR^{b}; and m is 1, 2, or 3. In some embodiments,

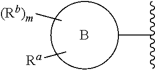

is

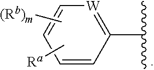

In some embodiments,

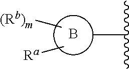

is

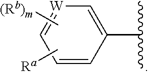

In some embodiments,

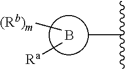

is

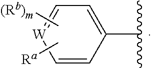

In some embodiments,

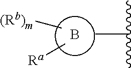

is

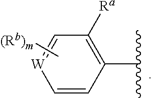

In some embodiments,

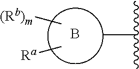

is

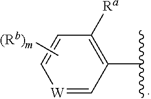

In some embodiments,

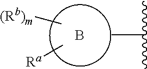

is

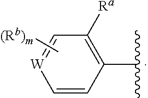

In some embodiments, W is N. In some embodiments, W is CR^{b}.

In some embodiments, the compound is a compound of Formula (XIV):

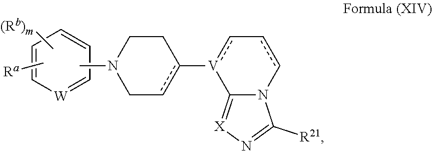

or a pharmaceutically acceptable salt, or solvate thereof, wherein W is N or CR^{b}; and m is 1, 2, or 3. In some embodiments, W is N. In some embodiments, W is CR^{b}.

In some embodiments, the compound is a compound of Formula (XIVa) or Formula (XIVb):

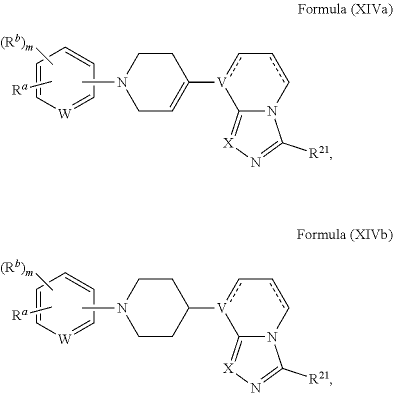

or a pharmaceutically acceptable salt, or solvate thereof, wherein W is N or CR^{b}; and m is 1, 2, or 3. In some embodiments, W is N. In some embodiments, W is CR^{b}.

In some embodiments, the compound is a compound of Formula (XIVa). In some embodiments, the compound is a compound of Formula (XIVb).

In some embodiments, the compound is a compound of Formula (XV):

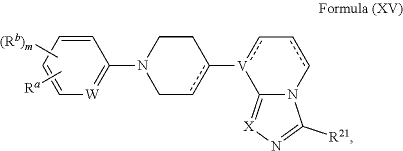

or a pharmaceutically acceptable salt, or solvate thereof, wherein W is N or CR^{b}; and m is 1, 2, or 3. In some embodiments, W is N. In some embodiments, W is CR^{b}.

In some embodiments, the compound is a compound of Formula (XVa) or Formula (XVb):

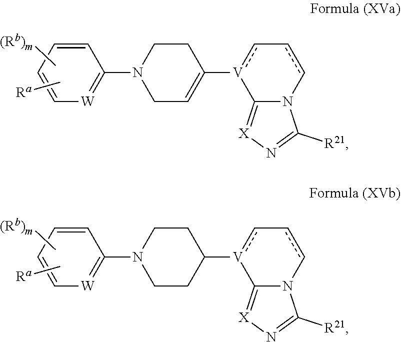

or a pharmaceutically acceptable salt, or solvate thereof, wherein W is N or CR^{b}; and m is 1, 2, or 3. In some embodiments, W is N. In some embodiments, W is CR^{b}.

In some embodiments, the compound is a compound of Formula (XVa). In some embodiments, the compound is a compound of Formula (XVb).

In some embodiments, the compound is a compound of Formula (XVI):

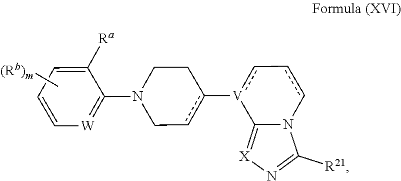

or a pharmaceutically acceptable salt, or solvate thereof, wherein W is N or CR^{b}; and m is 1, 2, or 3. In some embodiments, W is N. In some embodiments, W is CR^{b}.

In some embodiments, the compound is a compound of Formula (XVIa) or Formula (XVIb):

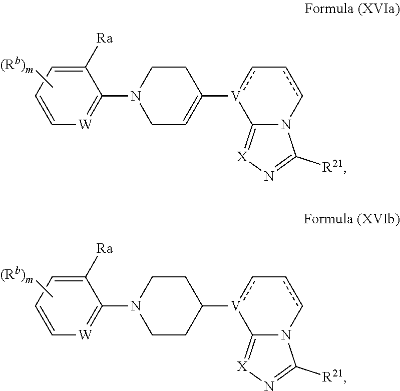

or a pharmaceutically acceptable salt, or solvate thereof, wherein W is N or CR^{b}; and m is 1, 2, or 3. In some embodiments, W is N. In some embodiments, W is CR^{b}.

In some embodiments, the compound is a compound of Formula (XVIa). In some embodiments, the compound is a compound of Formula (XVIb).

In some embodiments, R^{a} is selected from hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 4- to 8-membered heterocycloalkyl, unsubstituted or substituted phenyl, unsubstituted or substituted monocyclic heteroaryl, —CN, —OR²², —CO₂R²², —C(═O)N(R²²)₂, —N(R²²)₂, and —NR²²C(═O)R²³.

In some embodiments, R^{a} is selected from hydrogen, F, Cl, Br, —CN, —OH, —OCH₃, —OCH₂CH₃, —C(═O)CH₃, —C(═O)CH₂CH₃, —C(═O)OH, —C(═O)OCH₃, —C(═O)OCH₂CH₃, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CH₂CH(CH₃)₂, —CH₂F, —CHF₂, —CF₃, —NH₂, —NHCH₃, —N(CH₃)₂, —C(═O)NH₂, —C(═O)NHCH₃, —C(═O)N(CH₃)₂, cyclopropyl, cyclobutyl, cyclopentyl, cyclohexyl, unsubstituted or substituted phenyl, unsubstituted or substituted pyridinyl, unsubstituted or substituted pyrimidinyl, unsubstituted or substituted pyrazinyl, and unsubstituted or substituted pyridazinyl; wherein if R^{a} is substituted, it is substituted with one or more R^{s} groups independently selected from the group consisting of halogen, C₁-C₆ alkyl, —CN, —OR²⁴, —CO₂R²⁴, —C(═O)N(R²⁴)₂, —N(R²⁴)₂, —NR²⁴C(═O)R²⁵, and —SO₂R²⁵.

In some embodiments, R^{a} is selected from hydrogen, F, Cl, Br, —CN, —OH, —OCH₃, —OCH₂CH₃, —C(═O)CH₃, —C(═O)CH₂CH₃, —C(═O)OH, —C(═O)OCH₃, —C(═O)OCH₂CH₃, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CH₂CH(CH₃)₂, —CH₂F, —CHF₂, —CF₃, —NH₂, —NHCH₃, —N(CH₃)₂, —C(═O)NH₂, —C(═O)NHCH₃, —C(═O)N(CH₃)₂, cyclopropyl, cyclobutyl, cyclopentyl, and cyclohexyl. In some embodiments, R^{a} is selected from hydrogen, F, Cl, —CN, —OCH₃, —CH₃, —CH₂CH₃, —CH₂CH₂CH₃, —CH(CH₃)₂, —CH₂F, —CHF₂, and —CF₃. In some embodiments, R^{a} is selected from hydrogen, F, and —OCH₃.

In some embodiments, R^{a} is hydrogen.

In some embodiments, R^{a} is unsubstituted or substituted phenyl or unsubstituted or substituted monocyclic heteroaryl; wherein if R^{a} is substituted, it is substituted with one or more R^{s} groups independently selected from the group consisting of halogen, C₁-C₆ alkyl, —CN, —OR²⁴, —CO₂R²⁴, —C(═O)N(R²⁴)₂, —N(R²⁴)₂, —NR²⁴C(═O)R²⁵, or —SO₂R²⁵. In some embodiments, R^{a} is unsubstituted or substituted phenyl or unsubstituted or substituted 6-membered monocyclic heteroaryl; wherein if R^{a} is substituted, it is substituted with one or more R^{s} groups independently selected from the group consisting of halogen, C₁-C₆ alkyl, —CN, —OR²⁴, —CO₂R²⁴, —C(═O)N(R²⁴)₂, —N(R²⁴)₂, —NR²⁴C(═O)R²⁵, or —SO₂R²⁵. In some embodiments, R^{a} is unsubstituted or substituted phenyl or unsubstituted or substituted pyridinyl; wherein if R^{a} is substituted, it is substituted with one or more R^{s} groups independently selected from the group consisting of halogen, C₁-C₆ alkyl, —CN, —OR²⁴, —CO₂R²⁴, —C(═O)N(R²⁴)₂, —N(R²⁴)₂, —NR²⁴C(═O)R²⁵, or —SO₂R²⁵. In some embodiments, R^{a} is unsubstituted or substituted pyridinyl; wherein if R^{a} is substituted, it is substituted with one or more R^{s} groups independently selected from the group consisting of halogen, C₁-C₆ alkyl, —CN, —OR²⁴, —CO₂R²⁴, —C(═O)N(R²⁴)₂, —N(R²⁴)₂, —NR²⁴C(═O)R²⁵, or —SO₂R²⁵.

In some embodiments, each R^{b} is independently selected from hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 4- to 8-membered heterocycloalkyl, unsubstituted or substituted phenyl, unsubstituted or substituted monocyclic heteroaryl, —CN, —OR²², —CO₂R²², —C(═O)N(R²²)₂, —N(R²²)₂, and —NR²²C(═O)R²³.

In some embodiments, each R^{b} is independently selected from hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 4- to 8-membered heterocycloalkyl, —CN, —OR²², —CO₂R²², —C(═O)N(R²²)₂, —N(R²²)₂, and —NR²²C(═O)R²³.

In some embodiments, each R^{b} is independently selected from hydrogen, F, Cl, Br, —CN, —OH, —OCH₃, —OCH₂CH₃, —C(═O)CH₃, —C(═O)CH₂CH₃, —C(═O)OH, —C(═O)OCH₃, —C(═O)OCH₂CH₃, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CH₂CH(CH₃)₂, —CH₂F, —CHF₂, —CF₃, —NH₂, —NHCH₃, —N(CH₃)₂, —C(═O)NH₂, —C(═O)NHCH₃, —C(═O)N(CH₃)₂, cyclopropyl, cyclobutyl, cyclopentyl, and cyclohexyl. In some embodiments, each R^{b} is independently selected from hydrogen, F, Cl, —CN, —OCH₃, —CH₃, —CH₂CH₃, —CH₂CH₂CH₃, —CH(CH₃)₂, —CH₂F, —CHF₂, and —CF₃. In some embodiments, each R^{b} is independently selected from hydrogen, F, and —OCH₃.

In some embodiments, m is 1, 2, 3, or 4. In some embodiments, m is 1, 2, or 3. In some embodiments, m is 1 or 2. In some embodiments, m is 1. In some embodiments, m is 2. In some embodiments, m is 3. In some embodiments, m is 4.

In some embodiments, R^{a} is selected from hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 4- to 8-membered heterocycloalkyl, unsubstituted or substituted phenyl, unsubstituted or substituted monocyclic heteroaryl, —CN, —OR²², —CO₂R²², —C(═O)N(R²²)₂, —N(R²²)₂, and —NR²²C(═O)R²³; and each R^{b} is independently selected from hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 4- to 8-membered heterocycloalkyl, unsubstituted or substituted phenyl, unsubstituted or substituted monocyclic heteroaryl, —CN, —OR²², —CO₂R²², —C(═O)N(R²²)₂, —N(R²²)₂, and —NR²²C(═O)R²³.

In some embodiments, R^{a} is selected from hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 4- to 8-membered heterocycloalkyl, unsubstituted or substituted phenyl, unsubstituted or substituted monocyclic heteroaryl, —CN, —OR²², —CO₂R²², —C(═O)N(R²²)₂, —N(R²²)₂, and —NR²²C(═O)R²³; and each R^{b} is independently selected from hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 4- to 8-membered heterocycloalkyl, —CN, —OR²², —CO₂R²², —C(═O)N(R²²)₂, —N(R²²)₂, and —NR²²C(═O)R²³.

In some embodiments, R^{a} is selected from hydrogen, F, Cl, Br, —CN, —OH, —OCH₃, —OCH₂CH₃, —C(═O)CH₃, —C(═O)CH₂CH₃, —C(═O)OH, —C(═O)OCH₃, —C(═O)OCH₂CH₃, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CH₂CH(CH₃)₂, —CH₂F, —CHF₂, —CF₃, —NH₂, —NHCH₃, —N(CH₃)₂, —C(═O)NH₂, —C(═O)NHCH₃, —C(═O)N(CH₃)₂, cyclopropyl, cyclobutyl, cyclopentyl, cyclohexyl, unsubstituted or substituted phenyl, unsubstituted or substituted pyridinyl, unsubstituted or substituted pyrimidinyl, unsubstituted or substituted pyrazinyl, and unsubstituted or substituted pyridazinyl; wherein if R^{a} is substituted, it is substituted with one or more R^{s} groups independently selected from the group consisting of halogen, C₁-C₆ alkyl, —CN, —OR²⁴, —CO₂R²⁴, —C(═O)N(R²⁴)₂, —N(R²⁴)₂, —NR²⁴C(═O)R²⁵, and —SO₂R¹³; and each R^{b} is independently selected from hydrogen, F, Cl, Br, —CN, —OH, —OCH₃, —OCH₂CH₃, —C(═O)CH₃, —C(═O)CH₂CH₃, —C(═O)OH, —C(═O)OCH₃, —C(═O)OCH₂CH₃, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂CH₂CH(CH₃)₂, —CH₂F, —CHF₂, —CF₃, —NH₂, —NHCH₃, —N(CH₃)₂, —C(═O)NH₂, —C(═O)NHCH₃, —C(═O)N(CH₃)₂, cyclopropyl, cyclobutyl, cyclopentyl, cyclohexyl.

In some embodiments,

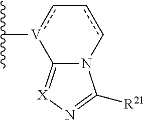

is

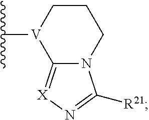

and V is CH or N. In some embodiments,

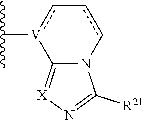

is

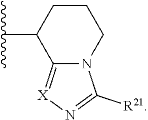

In some embodiments,

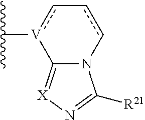

is

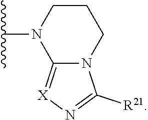

In some embodiments,

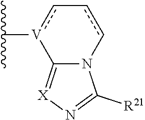

is

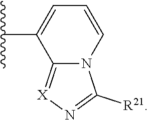

In some embodiments,

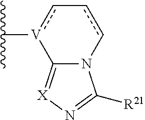

is

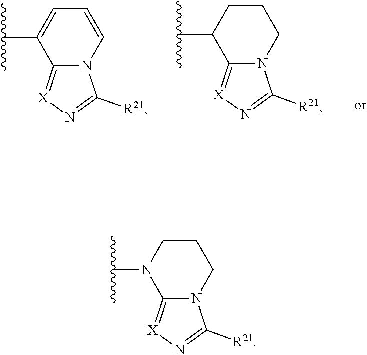

In some embodiments, X is N. In some embodiments, X is CH.

In some embodiments, R²¹ is —NH₂. In some embodiments, R²¹ is H.

In some embodiments,

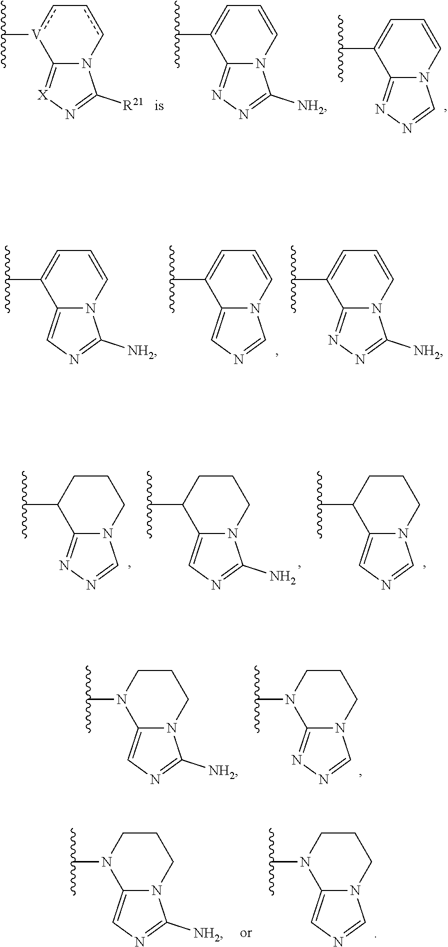

In some embodiments,

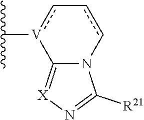

is

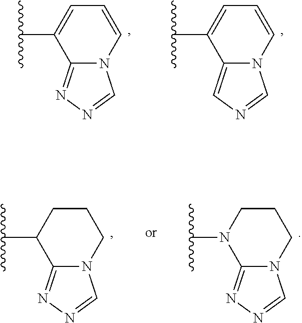 [SMILES: *c1cccn2cnnc12 ; *c1cccn2cncc12 ; *C1CCCn2cnnc21 ; *N1CCCn2cnnc21]

In some embodiments, the compound is a compound of the following formula:

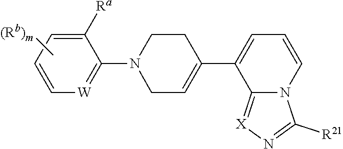

or a pharmaceutically acceptable salt, or solvate thereof, wherein W is N or CR^{b}; and m is 1, 2, or 3. In some embodiments, W is N. In some embodiments, W is CR^{b}.

In some embodiments, the compound is a compound of the following formula:

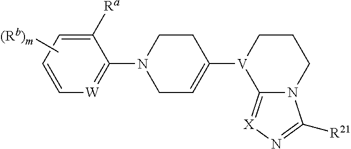

or a pharmaceutically acceptable salt, or solvate thereof, wherein V is N or CH; W is N or CR^{b}; and m is 1, 2, or 3. In some embodiments, W is N. In some embodiments, W is CR^{b}.

In some embodiments, the compound is a compound of the following formula:

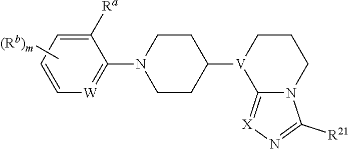

or a pharmaceutically acceptable salt, or solvate thereof, wherein Y is N or CH; W is N or CR^{b}; and m is 1, 2, or 3. In some embodiments, W is N. In some embodiments, W is CR^{b}.

In some embodiments, compounds described herein have the following structure:

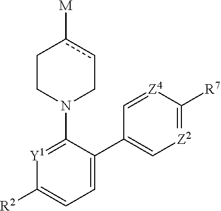

wherein M, [symbol] , Y¹, R², Z², R⁷, and Z⁴ are as described in Table 1.

In some embodiments, M is,

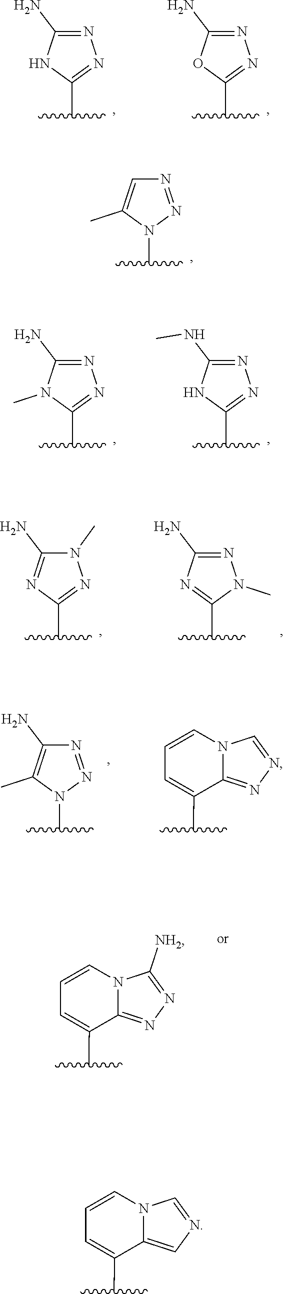

In some embodiments, Y¹ is N. In some embodiments, R² is H, F, or CH₃. In some embodiments, Z₂ is N or CF. In some embodiments, R⁷ is H, F, Cl, CN, NH₂, OCH₃, or OCH₂CH₂OCH₃. In some embodiments, Z⁴ is CH or N.

In some embodiments, compounds described herein have the following structure:

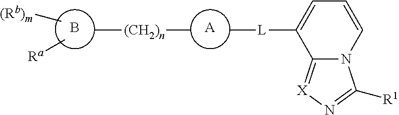

wherein R¹, X, L, Ring A, n, and

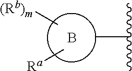

are as described in Table 1.

In some embodiments, compounds described herein have the following structure:

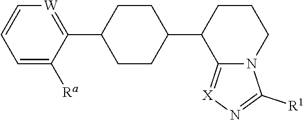

wherein R¹, X, W, and R^{a} are as described in Table 2.

Any combination of the groups described above for the various variables is contemplated herein. Throughout the specification, groups and substituents thereof are chosen by one skilled in the field to provide stable moieties and compounds.

Exemplary compounds described herein include the compounds described in the following Tables:

TABLE 1

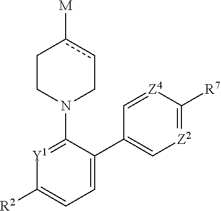

| Compd No. | M | [symbol] | Y¹ | R² | Z² | R⁷ | Z⁴ |
|---|---|---|---|---|---|---|---|
| 1 | 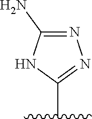 [SMILES: *c1nnc(N)[nH]1] | single bond | N | H | N | F | CH |
| 2 | 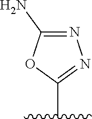 [SMILES: *c1nnc(N)o1] | single bond | N | H | N | F | CH |
| 3 | 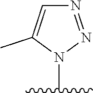 [SMILES: *n1nncc1C] | single bond | N | H | N | Cl | CH |
| 4 | 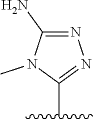 [SMILES: *c1nnc(N)n1C] | single bond | N | CH₃ | N | F | CH |
| 5 | 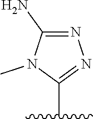 [SMILES: *c1nnc(N)n1C] | single bond | N | F | N | F | CH |
| 6 | 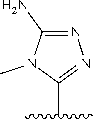 [SMILES: *c1nnc(N)n1C] | single bond | N | H | N | F | CH |
| 7 | 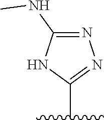 [SMILES: *c1nnc(NC)[nH]1] | single bond | N | H | N | F | CH |
| 8 | 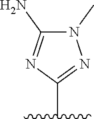 [SMILES: *c1nc(N)n(C)n1] | single bond | N | H | N | F | CH |
| 9 | 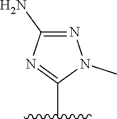 [SMILES: *c1nc(N)nn1C] | single bond | N | H | N | F | CH |
| 10 | 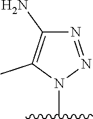 [SMILES: *n1nnc(N)c1C] | single bond | N | H | N | F | CH |
| 11 | 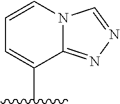 [SMILES: *c1cccn2cnnc12] | double bond | N | H | CF | F | CH |
| 12 | 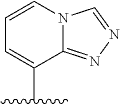 [SMILES: *c1cccn2cnnc12] | double bond | N | H | N | CN | CH |
| 13 | 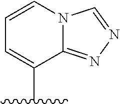 [SMILES: *c1cccn2cnnc12] | double bond | N | H | N | NH₂ | CH |
| 14 | 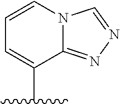 [SMILES: *c1cccn2cnnc12] | double bond | N | H | N | OCH₃ | CH |
| 15 | 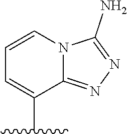 [SMILES: *c1cccn2c(N)nnc12] | double bond | N | H | N | F | CH |
| 16 | 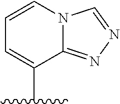 [SMILES: *c1cccn2cnnc12] | double bond | N | H | N | H | N |
| 17 | 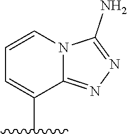 [SMILES: *c1cccn2c(N)nnc12] | single bond | N | H | N | F | CH |
| 18 | 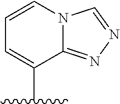 [SMILES: *c1cccn2cnnc12] | double bond | N | H | N | F | CH |
| 19 | 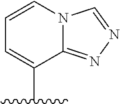 [SMILES: *c1cccn2cnnc12] | double bond | N | H | N | OCH₂CH₂OCH₃ | CH |
| 20 | 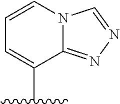 [SMILES: *c1cccn2cnnc12] | double bond | N | H | N | F | CH |

Compound in Table 1 are named:
1: 5-(1-(6′-fluoro-[3,3′-bipyridin]-2-yl)piperidin-4-yl)-4H-1,2,4-triazol-3-amine;
2: 5-(1-(6′-fluoro-[3,3′-bipyridin]-2-yl)piperidin-4-yl)-1,3,4-oxadiazol-2-amine;
3: 1-(1-(6′-chloro-[3,3′-bipyridin]-2-yl)piperidin-4-yl)-1H-1,2,3-triazol-4-amine;
4: 5-(1-(6′-fluoro-6-methyl-[3,3′-bipyridin]-2-yl)piperidin-4-yl)-4-methyl-4H-1,2,4-triazol-3-
amine;
5: 5-(1-(6,6′-difluoro-[3,3′-bipyridin]-2-yl)piperidin-4-yl)-4-methyl-4H-1,2,4-triazol-3-amine;
6: 5-(1-(6′-fluoro-[3,3′-bipyridin]-2-yl)piperidin-4-yl)-4-methyl-4H-1,2,4-triazol-3-amine;
7: 5-(1-(6′-fluoro-[3,3′-bipyridin]-2-yl)piperidin-4-yl)-N-methyl-4H-1,2,4-triazol-3-amine;
8: 3-(1-(6′-fluoro-[3,3′-bipyridin]-2-yl)piperidin-4-yl)-1-methyl-1H-1,2,4-triazol-5-amine;
9: 5-(1-(6′-fluoro-[3,3′-bipyridin]-2-yl)piperidin-4-yl)-1-methyl-1H-1,2,4-triazol-3-amine;
10: 1-(1-(6′-fluoro-[3,3′-bipyridin]-2-yl)piperidin-4-yl)-5-methyl-1H-1,2,3-triazol-4-amine;
11: 8-(3′-(3,4-difluorophenyl)-3,6-dihydro-2H-[1,2′-bipyridin]-4-yl)-[1,2,4]triazolo[4,3-
a]pyridine;
12: 4-([1,2,4]triazolo[4,3-a]pyridin-8-yl)-3,6-dihydro-2H-[1,2′:3′,3″-terpyridine]-6″-carbonitrile;
13: 4-([1,2,4]triazolo[4,3-a]pyridin-8-yl)-3,6-dihydro-2H-[1,2′:3′,3″-terpyridin]-6″-amine;
14: 8-(6″-methoxy-3,6-dihydro-2H-[1,2′:3′,3″-terpyridin]-4-yl)-[1,2,4]triazolo[4,3-a]pyridine;
15: 8-(6″-fluoro-3,6-dihydro-2H-[1,2′:3′,3″-terpyridin]-4-yl)-[1,2,4]triazolo[4,3-a]pyridin-3-
amine;
16: 8-(3′-(pyrimidin-5-yl)-3,6-dihydro-2H-[1,2′-bipyridin]-4-yl)-[1,2,4]triazolo[4,3-a]pyridine;
17: 8-(1-(6′-fluoro-[3,3′-bipyridin]-2-yl)piperidin-4-yl)-[1,2,4]triazolo[4,3-a]pyridin-3-amine;
18: 8-(6″-fluoro-3,6-dihydro-2H-[1,2′:3′,3″-terpyridin]-4-yl)-[1,2,4]triazolo[4,3-a]pyridine;
19: 8-(6″-(2-methoxyethoxy)-3,6-dihydro-2H-[1,2′:3′,3″-terpyridin]-4-yl)-[1,2,4]triazolo[4,3-
a]pyridine;
20: 8-(6″-fluoro-3,6-dihydro-2H-[1,2′:3′,3″-terpyridin]-4-yl)imidazo[1,5-a]pyridine.
In some embodiments, provided herein is a pharmaceutically acceptable salt of a
compound that is described in Table 1.

TABLE 2

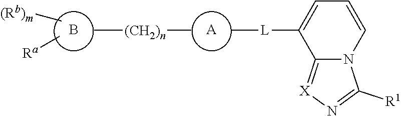

| Compd No. | 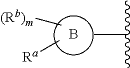 | n | 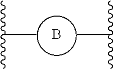 | L | X | R¹ |
|---|---|---|---|---|---|---|
| 21 | 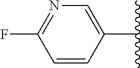 [SMILES: *c1ccc(F)nc1] | 1 | 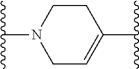 [SMILES: *C1=CCN(*)CC1] | absent | N | H |
| 22 | 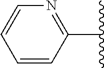 [SMILES: *c1ccccn1] | 0 | 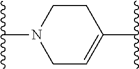 [SMILES: *C1=CCN(*)CC1] | absent | N | H |
| 23 | 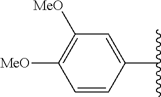 [SMILES: *c1ccc(OC)c(OC)c1] | 0 | 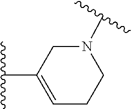 [SMILES: *C1=CCCN(*)C1] | 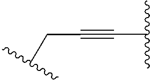 [SMILES: *C#CC*] | N | H |
| 24 | 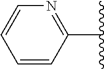 [SMILES: *c1ccccn1] | 0 | 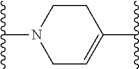 [SMILES: *C1=CCN(*)CC1] | absent | CH | H |
| 25 | 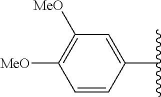 [SMILES: *c1ccc(OC)c(OC)c1] | 1 | 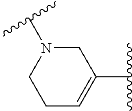 [SMILES: *C1=CCCN(*)C1] | absent | N | H |
| 26 | 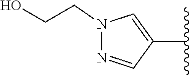 [SMILES: *c1cnn(CCO)c1] | 0 | 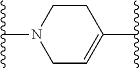 [SMILES: *C1=CCN(*)CC1] | absent | N | H |

Compounds in Table 1 are named:
21: 8-(1-((6-fluoropyridin-3-yl)methyl)-1,2,3,6-tetrahydropyridin-4-yl)-[1,2,4]triazolo[4,3-a]pyridine;
22: 8-(3,6-dihydro-2H-[1,2′-bipyridin]-4-yl)-[1,2,4]triazolo[4,3-]pyridine;
23: 8-(3-(5-(3,4-dimethoxyphenyl)-3,6-dihydropyridin-1(2H)-yl)prop-1-yn-1-yl)-[1,2,4]triazolo[4,3-a]pyridine;
24: 8-(3,6-dihydro-2H-[1,2′-bipyridin]-4-yl)imidazo[1,5-a]pyridine;
25: 8-(1-(3,4-dimethoxybenzyl)-1,2,5,6-tetrahydropyridin-3-yl)-[1,2,4]triazolo[4,3-a]pyridine;
26: 2-(4-(4-([1,2,4]triazolo[4,3-a]pyridin-8-yl)-3,6-dihydro-2H-[1,2′-bipyridin]-3-yl)-1H-pyrazol-1-yl)ethan-1-ol.
In some embodiments, provided herein is a pharmaceutically acceptable salt of acompound that is described in Table 2.

TABLE 3

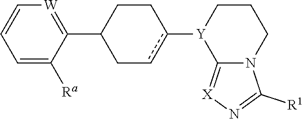

| Compd No. | R^{a} | [symbol] | Y | W | X | R¹ |
|---|---|---|---|---|---|---|
| 27 | H | single bond | CH | N | N | H |
| 28 | 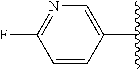 [SMILES: *c1ccc(F)nc1] | single bond | CH | N | N | H |
| 29 | 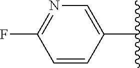 [SMILES: *c1ccc(F)nc1] | double bond | N | N | N | H |

Compounds in Table 3 are named:
27: 8-(1-(pyridin-2-yl)piperidin-4-y1)-5,6,7,8-tetrahydro-[1,2,4]triazolo[4,3-a]pyridine;
28: 8-(1-(6′-fluoro-[3,3′-bipyridin]-2-yl)piperidin-4-yl)-5,6,7,8-tetrahydro-[1,2,4]triazolo[4,3-a]pyridine;
29: 8-(6″-fluoro-3,6-dihydro-2H-[1,2′:3′,3″-terpyridin]-4-yl)-5,6,7,8-tetrahydro-[1,2,4]triazolo[4,3-a]pyrimidine.

In some embodiments, provided herein is a pharmaceutically acceptable salt of a compound that is described in Table 3.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic at the concentration or amount used, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid. In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-O-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with a base. In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds described herein, as well as active metabolites of these compounds having the same type of activity.

In some embodiments, sites on the organic radicals (e.g. alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds described herein possess one or more stereocenters and each stereocenter exists independently in either the R or S configuration. In some embodiments, the compound described herein exists in the R configuration. In some embodiments, the compound described herein exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound described herein are included within the scope of the claims. In some cases, some of the herein-described compounds is a prodrug for another derivative or active compound.

In some embodiments, any one of the hydroxyl group(s), amino group(s) and/or carboxylic acid group(s) are functionalized in a suitable manner to provide a prodrug moiety. In some embodiments, the prodrug moiety is as described above.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

In some instances, heterocyclic rings may exist in tautomeric forms. In such situations, it is understood that the structures of said compounds are illustrated or named in one tautomeric form but could be illustrated or named in the alternative tautomeric form. The alternative tautomeric forms are expressly included in this disclosure, such as, for example, the structures illustrated below. For example, benzimidazoles or imidazoles could exist in the following tautomeric forms:

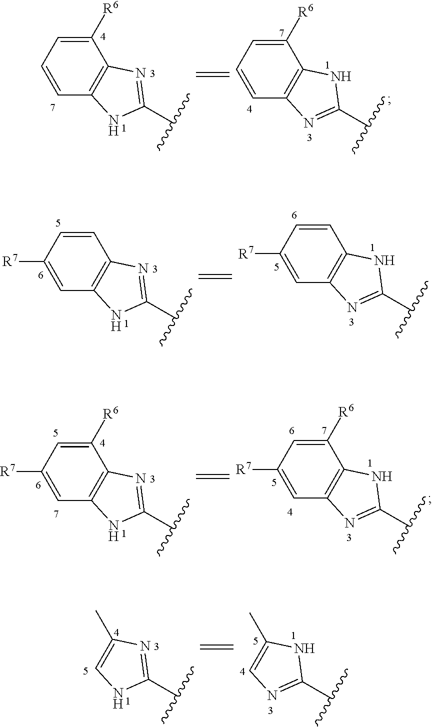

### Synthesis of Compounds

Compounds described herein are synthesized using standard synthetic techniques or using methods known in the art in combination with methods described herein.

Unless otherwise indicated, conventional methods of mass spectroscopy, NMR, IPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In some embodiments, compounds described herein are prepared as described in Scheme A.

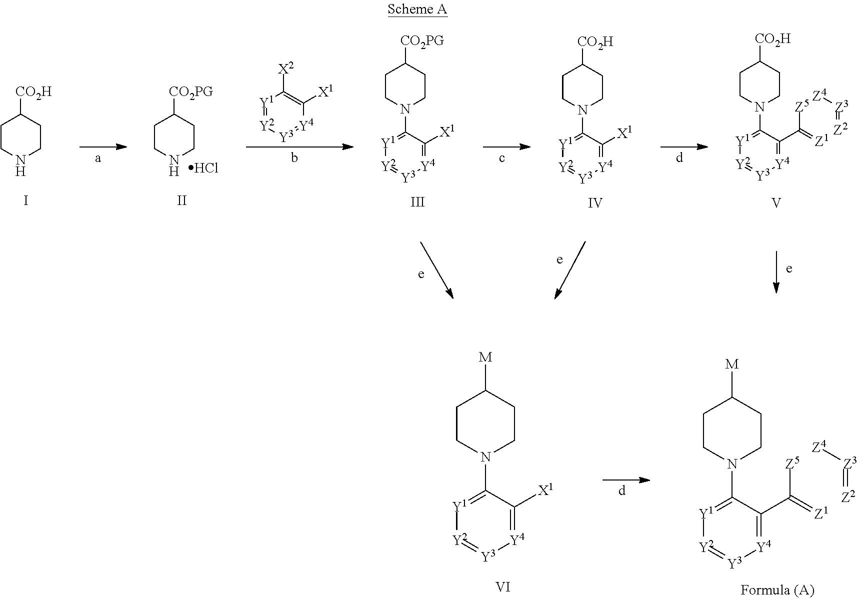

X¹ and X² are suitable leaving groups; PG is a suitable protecting group.

Protection of the acid I affords intermediate II, which was subsequently converted to intermediate III by a substitution reaction such as nucleophilic aromatic substitution (S_{N}Ar). Removal of the protecting group using appropriate de-protection methods yields intermediate IV. Intermediate IV is converted to intermediate V by an organometallic coupling reaction such as Suzuki-Miyaura reaction with a boronic acid or its ester or an organotrifluoroborate (Ar—BF₃K). Acid V is treated with suitable cyclization conditions to provide the compound of Formula (A). The reaction steps can proceed in different orders. For example, intermediate III or IV can be treated with suitable cyclization conditions to yield intermediate VI, which is subsequently converted to the compound of Formula (A) by an organometallic coupling reaction such as Suzuki-Miyaura reaction with a boronic acid or its ester or an organotrifluoroborate (Ar—BF₃K).

In some other embodiments, when M is a 1,2,3-trazole, compounds described herein are prepared as described in Scheme B.

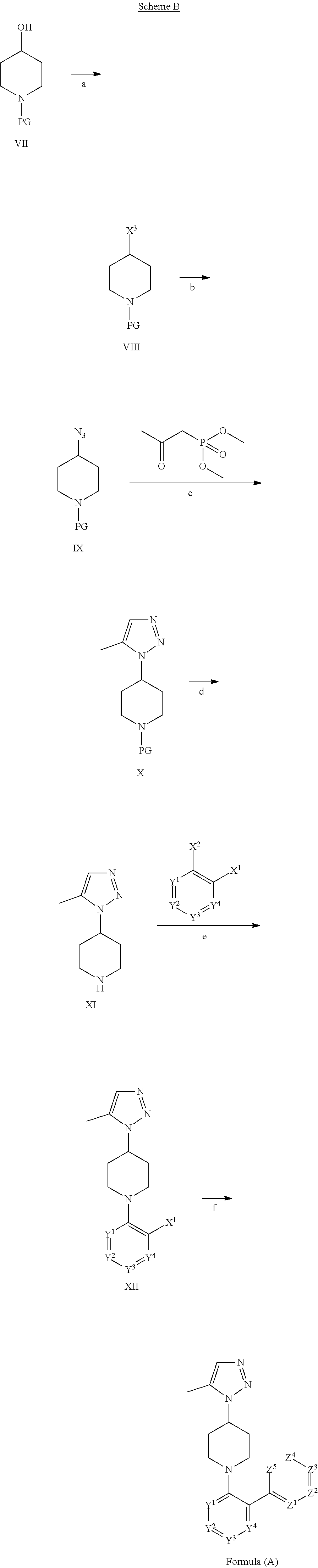

X¹ and X³ are suitable leaving groups; PG is a suitable protecting group.

The alcohol functional group of intermediate VII is converted to a suitable leaving group, such as a halide or sulfonate leavind group, resulting in intermediate VIII. Nucleophilic azide substitution results in intermediate IX, which is subsequently reacted with dimethyl (2-oxopropyl)phosphonate to yield intermediate X. Removal of the protecting group using appropriate de-protection methods yields intermediate XI. Intermediate XI is converted to intermediate XII by a substitution reaction such as a nucleophilic aromatic substitution (S_{N}Ar), which is subsequently converted to the compound of Formula (A) by an organometallic coupling reaction such as Suzuki-Miyaura reaction with a boronic acid or its ester or an organotrifluoroborate (Ar—BF₃K).

In some embodiments, compounds described herein are prepared as described in Scheme C.

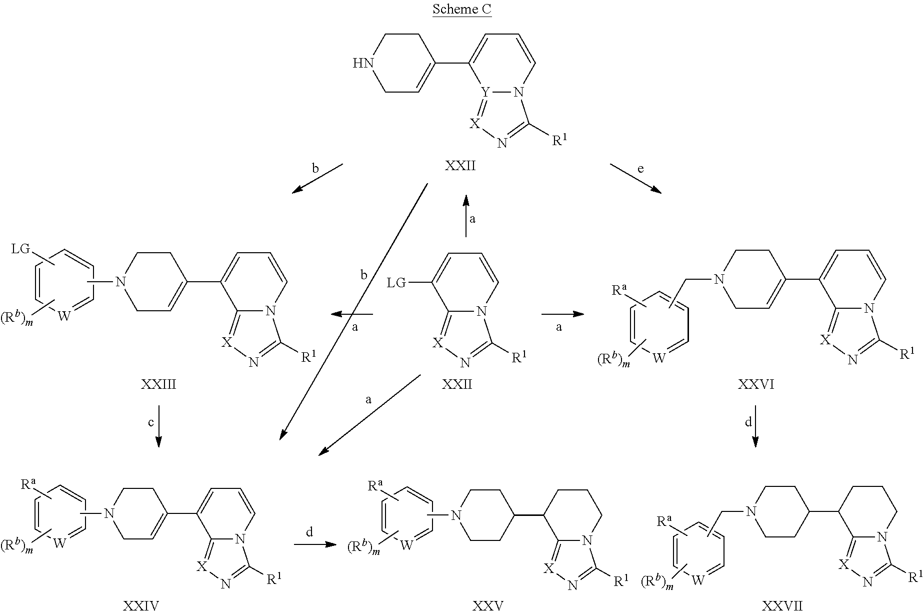

LG is a suitable leaving group, such as a halide or sulfonate.

Compound XXI is converted to intermediate XXII by an organometallic coupling reaction such as Suzuki-Miyaura reaction with the appropriate vinyl boronic acid or its ester or an organotrifluoroborate (BF₃K). Intermediate XXII can then be converted to Intermediate XXIII with an appropriate amination reaction, such as nucleophilic aromatic substitution (S_{N}Ar) or Buchwald-Hartwig amination. Intermediate XXIII is converted to Compound XXIV by an organometallic coupling reaction such as Suzuki-Miyaura reaction with the appropriate boronic acid or its ester or an organotrifluoroborate (BF₃K). Alternatively, Intermediate XXII can be converted directly to Compound XXIV with an appropriate amination reaction, such as nucleophilic aromatic substitution (S_{N}Ar) or Buchwald-Hartwig amination. Compound XXI can also be converted directly to Intermediate XXIII or Compound XXIV via an organometallic coupling reaction such as Suzuki-Miyaura reaction with the appropriate vinyl boronic acid or its ester or an organotrifluoroborate (BF₃K). Compound XXIV can be converted to Compound XXV by any suitable hydrogenation reaction, such as hydrogen gas (H₂) over a metal catalyst, such as palladium (Pd). Intermediate XXII is alternatively converted to Compound XXVI by substitution reaction with, for example, a suitably substituted benzyl halide or pyridinyl-methylbromide. Compound XXVI can be converted to Compound XXVII by any suitable hydrogenation reaction, such as hydrogen gas (H₂) over a metal catalyst, such as palladium (Pd).

In some embodiments, compounds are prepared as described in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

An “hydroxyalkyl” refers to an alkyl in which one hydrogen atom is replaced by a hydroxyl. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like.

An “aminoalkyl” refers to an alkyl in which one hydrogen atom is replaced by an amino. In some embodiments, aminoalkyl is a C₁-C₄aminoalkyl. Typical aminoalkyl groups include, but are not limited to, —CH₂NH₂, —CH₂CH₂NH₂, —CH₂CH₂CH₂NH₂, —CH₂CH₂CH₂CH₂NH₂, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “aromatic” refers to a planar ring having a delocalized π-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl. In some embodiments, a cycloalkyl is a C₃-C₄cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

In some embodiments, each substituted alkyl, substituted fluoroalkyl, substituted heteroalkyl, substituted carbocycle, and substituted heterocycle is substituted with one or more R^{s} groups independently selected from the group consisting of halogen, C₁-C₆alkyl, monocyclic carbocycle, monocyclic heterocycle, —CN, —OR²¹, —CO₂R²¹, —C(═O)N(R²¹)₂, —N(R²¹)₂, —NR²¹C(═O)R²², —SR²¹, —S(═O)R²², —SO₂R²², or —SO₂N(R²¹)₂; each R²¹ is independently selected from hydrogen, C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl; or two R²¹ groups are taken together with the N atom to which they are attached to form a N-containing heterocycle; each R²² is independently selected from C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist. In some embodiments, a modulator is an inhibitor.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development or progression of the disease or condition, relieving the disease or condition, causing regression of the disease or condition, relieving a secondary condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, for example compounds of Formula (A), Formula (I), and Formula (XI), or a pharmaceutically acceptable salt thereof, are used in the preparation of medicaments for the treatment of diseases or conditions in a mammal that would benefit from modulation of QPCTL activity. Methods for treating any of the diseases or conditions described herein in a mammal in need of such treatment, involves administration of pharmaceutical compositions that include at least one compound described herein, or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-2000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, described herein are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

### Combination Treatments

In certain instances, it is appropriate to administer at least one compound described herein, or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject.

### Examples

As used above, and throughout the description of the invention, the following abbreviations, unless otherwise indicated, shall be understood to have the following meanings:

### Abbreviations

Pd(OAc)₂: palladium(II) acetate;
P(o-Tol)₃: tri(ortho-tolyl)phosphine;
TFA: trifluoroacetic acid;
MeCN or CH₃CN or ACN: acetonitrile;
H₂O: water;
DMF: dimethylformamide;
DCM: dichloromethane;
rt: room temperature;
hrs: hours;
h or hr: hour;
min: minute;
N: normalily;
g: grams
mg: milligrams;
mL: milliliter;
Eq. or equiv: equivalents;
mmol: millimole;
EtOAc: ethyl acetate;
Na₂SO₄: sodium sulfate;
cat.: catalytic;
TBAB: tetra-n-butylammonium bromide;
THF: tetrahydrofuran;
DME: dimethoxyethane;
MeOH: methanol;
MsCl: methanesulfonyl chloride or mesyl chloride.

The following examples are provided for illustrative purposes only and not to limit the scope of the claims provided herein.

### Synthesis of Compounds

### Example 1. Preparation of 5-(1-(6′-fluoro-[3,3′-bipyridin]-2-yl)piperidin-4-yl)-4H-1,2,4-triazol-3-amine (Compound 1)

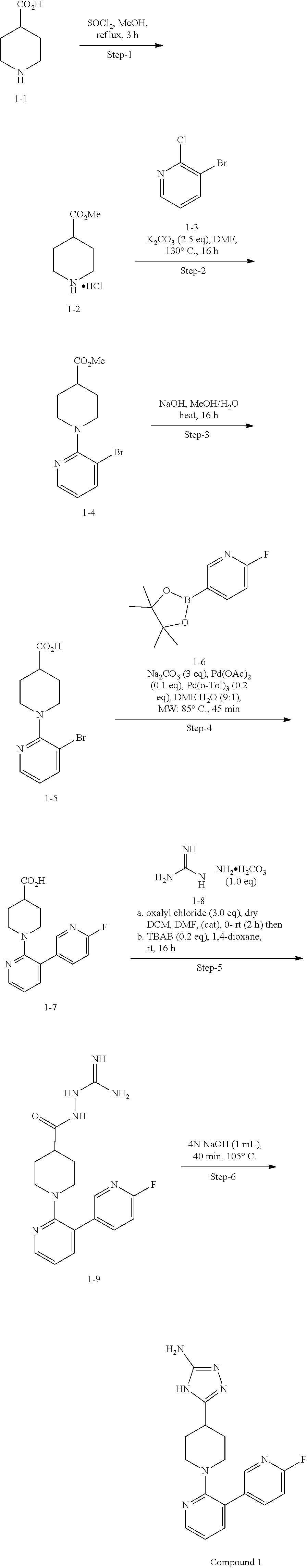

Step 1. methyl piperidine-4-carboxylate (1-2): Thionyl chloride (45 mmol) was added to a solution of 1-1 (30 mmol) in DCM (75 mL) and methanol was added and warmed from 0° C. to rt over 4 hours. Brine (50 mL) was added and then extracted with DCM (50 mL×3) and evaporated to dryness to give 1-2 which was used in the next step without further purification.

Step 2. methyl 1-(3-bromopyridin-2-yl)piperidine-4-carboxylate (1-4): To the 1-2 obtained in Step 1 was added DMF (15 mL) and 1-3 (36 mmol) followed by K₂CO₃ (90 mmol) and the mixture was heated at 140° C. for 16 hr. Brine (50 mL) was added and then extracted with EtOAc (50 mL×3). The organic layer was dried with Na₂SO₄, filtered, then concentrated to dryness to give 1-4, which was used in the next step without further purification.

Step 3. 1-(3-bromopyridin-2-yl)piperidine-4-carboxylic acid (1-5): To the 1-4 obtained in Step 2 was added methanol (15 mL) 1N sodium hydroxide was added and the mixture was heated for 16 hrs. Brine (50 mL) was added and then extracted with EtOAc (50 mL×3). The organic layer was dried with Na₂SO₄, filtered, then concentrated to dryness to give 1-5, which was used in the next step without further purification.

Step 4. 1-(6′-fluoro-[3,3′-bipyridin]-2-yl)piperidine-4-carboxylic acid (1-7): Pd(OAc)₂ (0.1 equiv), P(o-Tol)₃ (0.2 equiv), Na₂CO₃ (2.0 equiv), DME, and water (9:1) were added to 1-5 obtained in Step 5 and the mixture was heated at 85° C. for 45 minutes under microwave conditions. To this was then added brine (50 mL) then extracted with EtOAc (50 mL×3). The organic layer was dried with Na₂SO₄, filtered, then concentrated and purified via reverse phase chromatography eluting 5-100% ACN (+0.1% NH₄OH) in H₂O (+0.1% NH₄OH) to obtain Compound 3.

Step 5. 2-(1-(6′-fluoro-[3,3′-bipyridin]-2-yl)piperidine-4-carbonyl)hydrazine-1-carboximidamide (1-9): A solution of 1-7 (1.0 g, 3.3 mmol), oxalyl chloride (10 mmol), DCM (10 mL), cat. DMF, and hydrazinecarboximidamide (1-8, 3.3 mmol) and was allowed to warm to rt from 0° C. over 2 hours. TBAB (0.66 mmol) was added, followed by 1,4-dioxane and the mixture was stirred at rt for 16 hr to give 1-9, which was used in the next step without purification.

Step 6. 5-(1-(6′-fluoro-[3,3′-bipyridin]-2-yl)piperidin-4-yl)-4H-1,2,4-triazol-3-amine (Compound 1): To the solution of 1-9 obtained in Step 5 was added 1N NaOH (1.0 mL) and the mixture was heated at 105° C. for 40 minutes. To this was then added brine (50 mL) then extracted with EtOAc (50 mL×3). The organic layer was dried with Na₂SO₄, filtered, then concentrated and purified via reverse phase chromatography eluting 5-100% ACN (+0.1% NH₄OH) in H₂O (+0.1% NH₄OH) to obtain Compound 1. M/Z: 340 [M+H]⁺.

### Example 2. Preparation of 5-(1-(6′-fluoro-[3,3′-bipyridin]-2-yl)piperidin-4-yl)-1,3,4-oxadiazol-2-amine (Compound 2)

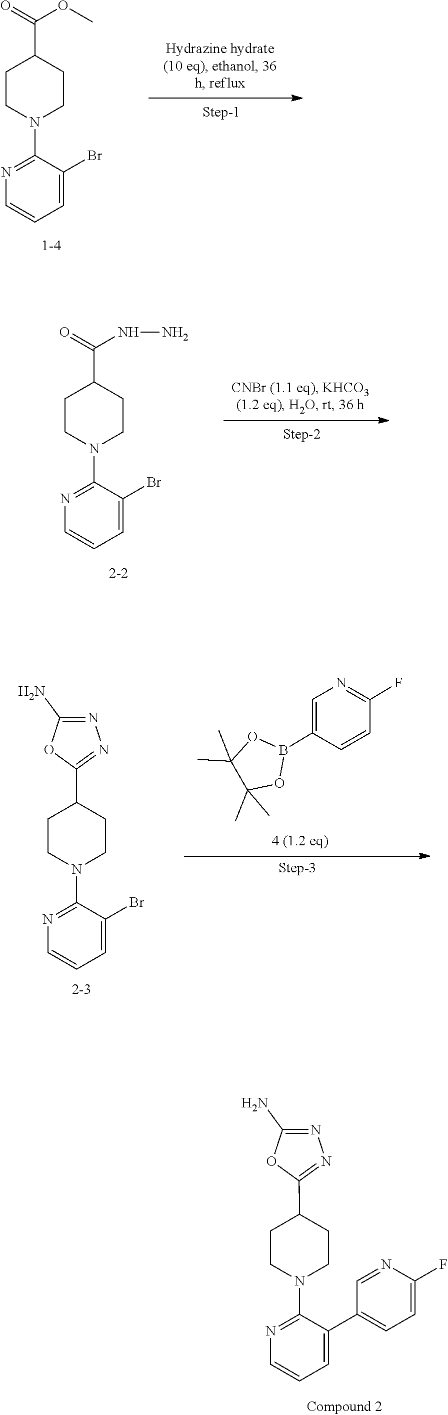 [SMILES: COC(=O)C1CCN(c2ncccc2Br)CC1 ; NNC(=O)C1CCN(c2ncccc2Br)CC1 ; Nc1nnc(C2CCN(c3ncccc3Br)CC2)o1 ; CC1(C)OB(c2ccc(F)nc2)OC1(C)C ; Nc1nnc(C2CCN(c3ncccc3-c3ccc(F)nc3)CC2)o1]

Step 1. Compound name (2-2): Hydrazine hydrate (10 equiv) was added to 1-4 (1.0 g, 3.35 mmol) in EtOH (30 mL) and the mixture was refluxed for 36 hours. The EtOH was evaporated and brine (50 mL) was added and then extracted with EtOAc (50 mL×3). The organic layer was dried with Na₂SO₄, filtered, then concentrated to dryness to give 2-2, which was used in the next step without further purification.

Step 2. Compound name (2-3): Cyanogen bromide (1.1 equiv), KHCO₃ (1.2 equiv), and water (10 mL) were added to 2-2 obtained in Step 1 and the mixture was stirred for 36 hr at rt. Brine (50 mL) was added and then extracted with EtOAc (50 mL×3). The organic layer was dried with Na₂SO₄, filtered, then concentrated to dryness to give to give 2-3, which was used in the next step without further purification.

Step 3: 5-(1-(6′-fluoro-[3,3′-bipyridin]-2-yl)piperidin-4-yl)-1,3,4-oxadiazol-2-amine (Compound 2): Pd(OAc)₂ (0.1 equiv), P(o-Tol)₃ (0.2 equiv), Na₂CO₃ (2.0 equiv), DME, and water (9:1) were added to 2-3 obtained in Step 2 and heated at 85° C. for 45 minutes under microwave conditions. To this was then added brine (50 mL) then extracted with EtOAc (50 mL×3). The organic layer was dried with Na₂SO₄, filtered, then concentrated and purified via reverse phase chromatography eluting 5-100% ACN (+0.1% NH₄OH) in H₂O (+0.1% NH₄OH) to obtain Compound 2. M/Z: 341 [M+H]⁺.

### Example 3. Preparation of 1-(1-(6′-chloro-[3,3′-bipyridin]-2-yl)piperidin-4-yl)-1H-1,2,3-triazol-4-amine (Compound 3)

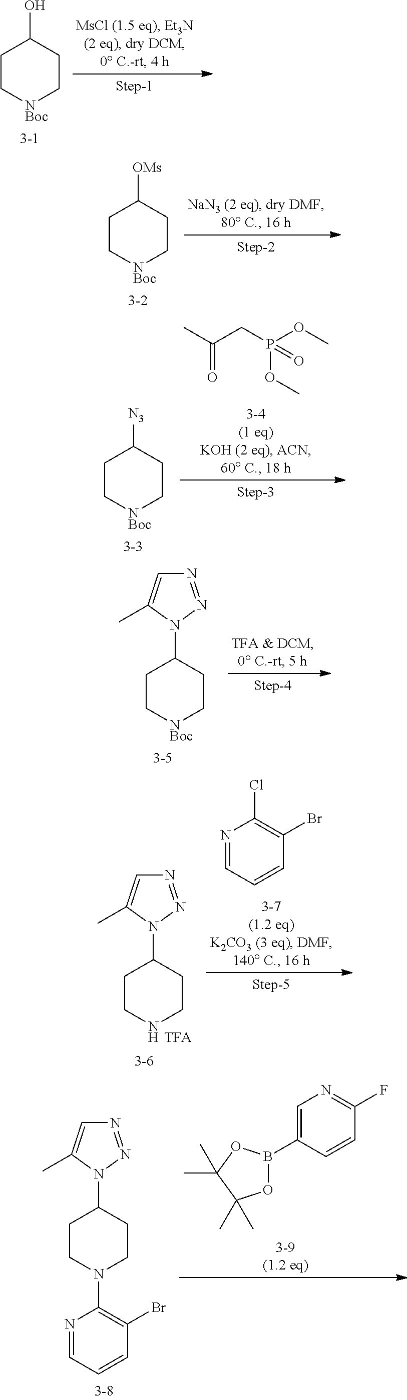

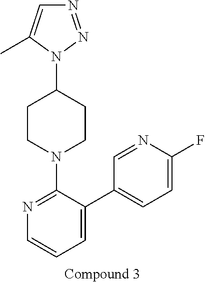 [SMILES: Cc1cnnn1C1CCN(c2ncccc2-c2ccc(F)nc2)CC1]

Step 1. Compound name (3-2): Mesyl chloride (45 mmol) was added to a solution of tert-butyl 4-hydroxypiperidine-1-carboxylate (3-1, 6.0 g, 30 mmol) in DCM (75 mL) and triethylamine (60 mmol) and the mixture was warmed from 0° C. to rt over 4 hours. Brine (50 mL) was added and then extracted with DCM (50 mL×3) and evaporated to dryness to give 3-2, which was used in the next step without further purification.

Step 2. compound name (3-3): Anhydrous DMF (30 mL) was added to 3-2 obtained in Step 1, followed by sodium azide (60 mmol) and the mixture was heated at 80° C. for 16 hr. Brine (50 mL) was added and then extracted with EtOAc (50 mL×3). The organic layer was dried with Na₂SO₄, filtered, then concentrated to dryness to give to give to give 3-3, which was used in the next step without further purification.

Step 3. Compound name (3-5): 3-3 was dissolved in acetonitrile (30 mL) and dimethyl (2-oxopropyl)phosphonate (3-4, 30 mmol) and KOH (60 mmol) was added and the mixture was heated at 60° C. for 16 hr. Brine (50 mL) was added and then extracted with EtOAc (50 mL×3). The organic layer was dried with Na₂SO₄, filtered, then concentrated to dryness to give 3-5, which was used in the next step without further purification.

Step 4. Compound name (3-6): 3-5 was dissolved in DCM (30 mL) and TFA was added (3.0 mL) and the mixture was allowed to warm from 0° C. to rt over 4 hr. The solution was concentrated to dryness to give 3-6, which was used in the next step without further purification.

Step 5. Compound name (3-8): To the 3-6 obtained in Step 4 was added DMF (15 mL) and 2-chloro-3-bromopyridine (36 mmol) followed by K₂CO₃ (90 mmol) and the mixture was heated at 140° C. for 16 hr. Brine (50 mL) was added and then extracted with EtOAc (50 mL×3). The organic layer was dried with Na₂SO₄, filtered, then concentrated to dryness to give 3-8, which was used in the next step without further purification.

Step 6. 1-(1-(6′-chloro-[3,3′-bipyridin]-2-yl)piperidin-4-yl)-1H-1,2,3-triazol-4-amine (Compound 3): Pd(OAc)₂ (0.1 equiv), P(o-Tol)₃ (0.2 equiv), Na₂CO₃ (2.0 equiv), DME, and water (9:1) were added to 3-8 obtained in Step 5 and the mixture was heated at 85° C. for 45 minutes under microwave conditions. To this was then added brine (50 mL) then extracted with EtOAc (50 mL×3). The organic layer was dried with Na₂SO₄, filtered, then concentrated and purified via reverse phase chromatography eluting 5-100% ACN (+0.1% NH₄OH) in H₂O (+0.1% NH₄OH) to obtain Compound 3. M/Z: 355 [M+H]⁺.

### Example 4. Preparation of 8-(1-((6-fluoropyridin-3-yl)methyl)-1,2,3,6-tetrahydropyridin-4-yl)-[1,2,4]triazolo[4,3-a]pyridine (Compound 21)

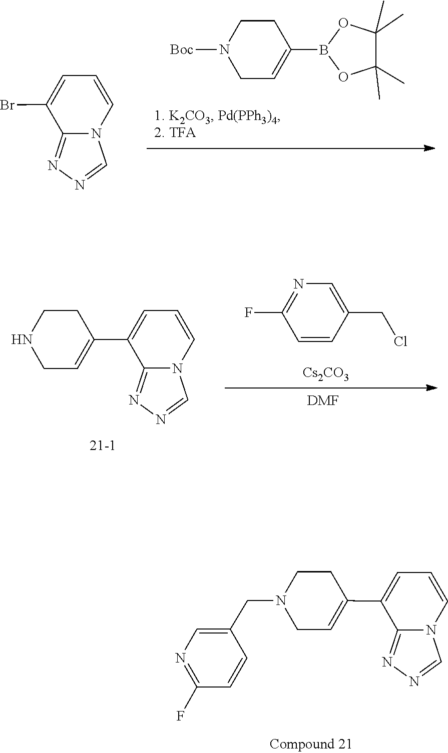

Step 1: 8-(1,2,3,6-Tetrahydropyridin-4-yl)-[1,2,4]triazolo[4,3-a]pyridine (21-1). To a solution of 8-bromo-[1,2,4]triazolo[4,3-a]pyridine (0.500 g, 2.52 mmol) in dioxane:water (10:1, 11 mL) was added tert-butyl 4-(4,4,5,5-tetramethyl-1,3,2-dioxaborolan-2-yl)-3,6-dihydropyridine-1(2H)-carboxylate (1.17 g, 3.79 mmol), K₂CO₃ (0.698 g, 5.05 mmol) and Pd(PPh₃)₄ (0.146 g, 0.126 mmol) and the mixture was heated to reflux overnight. To this was then added brine (50 mL) then extracted with EtOAc (50 mL×3). The organic layer was dried with Na₂SO₄, filtered, then concentrated and purified via silica gel chromatography eluting 0-100% EtOAc in hexanes followed by 0-20% MeOH in CH₂Cl₂. The product was isolated as a pink solid. The product was then concentrated and dissolved in CH₂Cl₂ (10 mL) and then added TFA (3 mL) and stirred at room temperature for ˜1 h. To the reaction mixture was added sat. NaHCO₃ (aq) (50 mL) then extracted with CH₂Cl₂ (50 mL×3). The organic layers were isolated and concentrated to yield 21-1 as a yellow solid. Yield=0.352 g (69%). M/Z: 201 [M+H]⁺.

Step 2: 8-(1-((6-Fluoropyridin-3-yl)methyl)-1,2,3,6-tetrahydropyridin-4-yl)-[1,2,4]triazolo[4,3-a]pyridine (Compound 21). A solution of 21-1 (0.2 g, 1 mmol), 5-(chloromethyl)-2-fluoropyridine (0.174 g, 1.20 mmol) and Cs₂CO₃ (0.651 g, 2 mmol) in DMF (3 mL) was heated to 90° C. overnight. The reaction mixture was concentrated, filtered, then purified via prep HPLC eluting 5-95% ACN (+0.1% NH₄OH) in water (+0.1% NH₄OH) to yield Compound 21. Yield=0.17 g (55%). M/Z: 310 [M+H]⁺.

### Example 5. Preparation of 8-(3,6-dihydro-2H-[1,2′-bipyridin]-4-yl)-[1,2,4]triazolo[4,3-a]pyridine (Compound 22) and 8-(1-(pyridin-2-yl)piperidin-4-yl)-5,6,7,8-tetrahydro-[1,2,4]triazolo[4,3-a]pyridine (Compound 27)

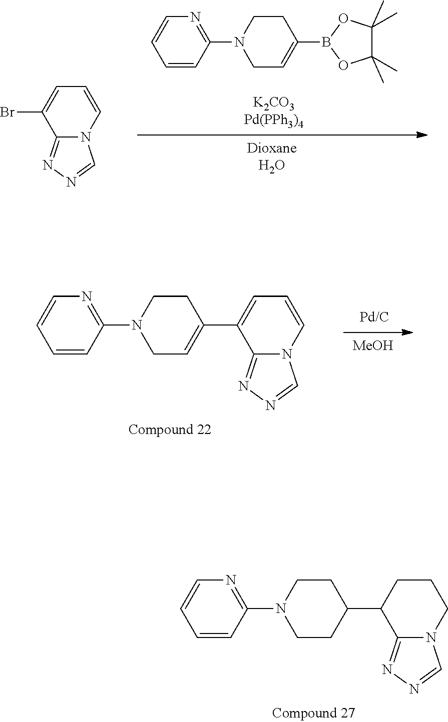 [SMILES: "MeOH" ; CC1(C)OB(C2=CCN(c3ccccn3)CC2)OC1(C)C ; Brc1cccn2cnnc12 ; C1=C(c2cccn3cnnc23)CCN(c2ccccn2)C1 ; c1ccc(N2CCC(C3CCCn4cnnc43)CC2)nc1]

Step 1: 8-(3,6-Dihydro-2H-[1,2′-bipyridin]-4-yl)-[1,2,4]triazolo[4,3-a]pyridine (Compound 22). A solution of 8-bromo-[1,2,4]triazolo[4,3-a]pyridine (0.208 g, 1.05 mmol), 4-(4,4,5,5-tetramethyl-1,3,2-dioxaborolan-2-yl)-3,6-dihydro-2H-1,2′-bipyridine (0.25 g, 0.874 mmol), K₂CO₃ (0.241 g, 1.75 mmol) and Pd(PPh₃)₄ (0.050 g, 43.7 mmol) in dioxane (10 mL) Water (1.1 mL) was heated to reflux for 3 hr. To this was then added brine (50 mL) then extracted with EtOAc (50 mL×3). The organic layer was dried with Na₂SO₄, filtered, then concentrated and purified via reverse phase chromatography eluting 5-100% ACN (+0.1% NH₄OH) in H₂O (+0.1% NH₄OH) to yield Compound 22. Yield=0.18 g (74%). M/Z: 278 [M+H]+.

Step 2: 8-(3,6-dihydro-2H-[1,2′-bipyridin]-4-yl)-[1,2,4]triazolo[4,3-a]pyridine (Compound 27). A solution of Compound 22 (0.040 g, 0.14 mmol) in MeOH (10 mL) was added Pd/C (0.015 g, 0.14 mmol) and placed under vacuum for ˜30 min. The reaction mixture was then purged with hydrogen and allowed to stir at room temperature for ˜2 hr. The reaction mixture was filtered through celite, then concentrated and purified via prep HPLC eluting 5-95% ACN (+0.1% NH₄OH) in H₂O (+0.1% NH₄OH) to yield Compound 27. Yield=0.02 g (52%). M/Z: 280 [M+H]+.

### Example 6. Preparation of 8-(3-(5-(3,4-dimethoxyphenyl)-3,6-dihydropyridin-1(2H)-yl)prop-1-yn-1-yl)-[1,2,4]triazolo[4,3-a]pyridine (Compound 23)

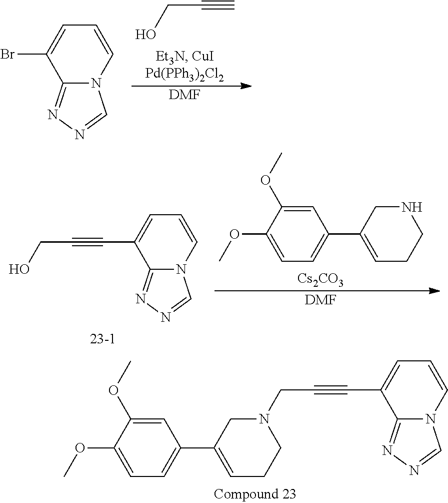

Step 1: 3-([1,2,4]Triazolo[4,3-a]pyridin-8-yl)prop-2-yn-1-ol (23-1). To a pressure vessel was added 8-bromo-[1,2,4]triazolo[4,3-a]pyridine (0.500 g, 2.52 mmol), propargyl alcohol (0.283 g, 5.05 mmol), Et₃N (0.767 g, 7.57 mmol), CuI (0.024 g, 0.126 mmol) and Pd(PPh₃)₂Cl₂ (0.177 g, 0.252 mmol) and purged with nitrogen then sealed and heated to 130° C. for 60 mins. The reaction mixture was then filtered through celite, concentrated and purified via reverse phase chromatography eluting 0-35% ACN (+0.1% NH₄OH) in H₂O (+0.1% NH₄OH) to yield 23-1. Yield=0.4 g (92%). M/Z: 174 [M+H]⁺.

Step 2: 8-(3-(5-(3,4-Dimethoxyphenyl)-3,6-dihydropyridin-1(2H)-yl)prop-1-yn-1-yl)-[1,2,4]triazolo[4,3-a] pyridine (Compound 23). To a solution of 23-1 (0.21 g, 1.19 mmol) and Et₃N (0.493 mL, 3.56 mmol) in CH₂Cl₂ (20 mL) was added MsCl (0.136 mL, 1.78 mL) at 0° C. and allowed to stir for 1 hr. To this was then added sat. NaHCO₃ (aq., 50 mL) then extracted with CH₂Cl₂ (50 mL×3). The organic layer was dried with Na₂SO₄, filtered, then concentrated to provide crude 3-([1,2,4]triazolo[4,3-a]pyridin-8-yl)prop-2-yn-1-yl methanesulfonate which was used without further purification.

A solution of the crude 3-([1,2,4]triazolo[4,3-a]pyridin-8-yl)prop-2-yn-1-yl methanesulfonate (0.300 g, 1.19 mmol), 5-(3,4-dimethoxyphenyl)-1,2,3,6-tetrahydropyridine (0.314 g, 1.43 mmol), and Cs₂CO₃ (0.778 g, 2.39 mmol) in DMF (5 mL) was heated to 90° C. overnight. The reaction mixture was concentrated, filtered then purified via prep HPLC eluting 5-95% ACN (+0.1 NH₄OH) in H₂O (+0.1 NH₄OH) to yield Compound 23. Yield=0.021 g (5%). M/Z: 375 [M+H]+.

### Example 7. Preparation of 8-(3,6-dihydro-2H-[1,2′-bipyridin]-4-yl)imidazo[1,5-a]pyridine (Compound 24)

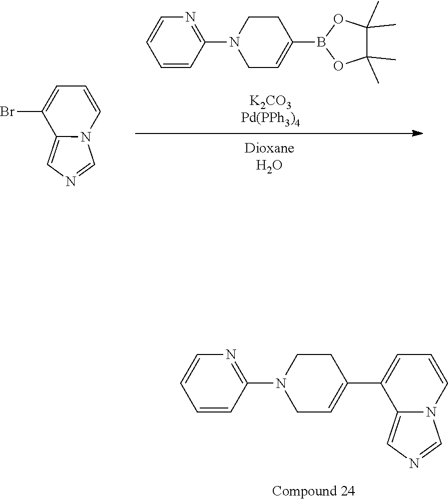 [SMILES: CC1(C)OB(C2=CCN(c3ccccn3)CC2)OC1(C)C ; Brc1cccn2cncc12 ; C1=C(c2cccn3cncc23)CCN(c2ccccn2)C1]

A solution of 8-bromoimidazo[1,5-a]pyridine (0.2 g, 1.02 mmol), 4-(4,4,5,5-tetramethyl-1,3,2-dioxaborolan-2-yl)-3,6-dihydro-2H-1,2′-bipyridine (0.25 g, 0.874 mmol), K₂CO₃ (0.241 g, 1.75 mmol) and Pd(PPh₃)₄ (0.050 g, 43.7 mmol) in dioxane (10 mL) Water (1.1 mL) was heated to reflux for 3 hr. To this was then added brine (50 mL) then extracted with EtOAc (50 mL×3). The organic layer was dried with Na₂SO₄, filtered, then concentrated and purified via reverse phase chromatography eluting 5-100% ACN (+0.1% NH₄OH) in H₂O (+0.1% NH₄OH) to yield Compound 24. Yield=0.112 g (47%). M/Z: 277 [M+H]⁺.

### Example 8. Preparation of 8-(1-(6′-fluoro-[3,3′-bipyridin]-2-yl)piperidin-4-yl)-5,6,7,8-tetrahydro-[1,2,4]triazolo[4,3-a]pyridine (Compound 28)

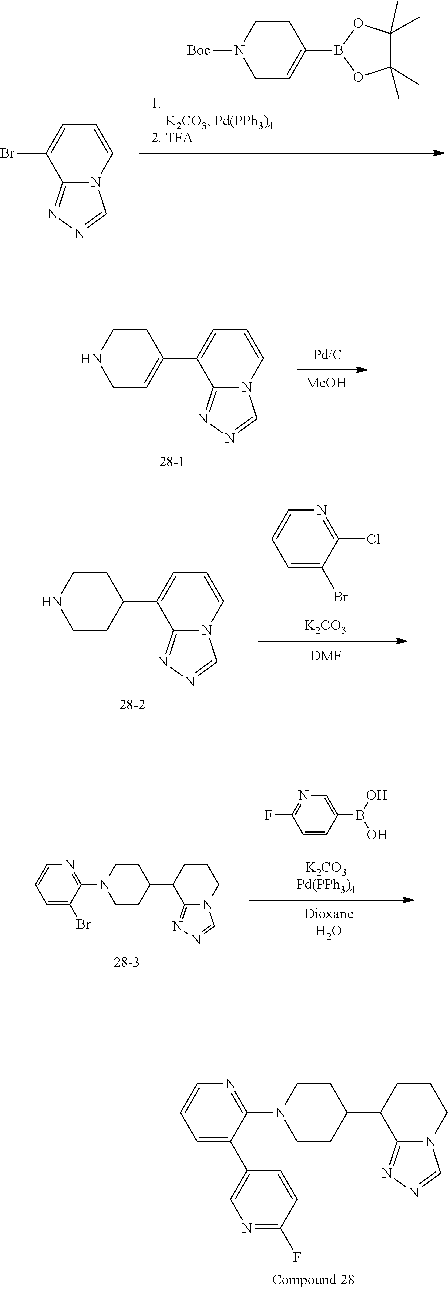

Step 1: 8-(1,2,3,6-Tetrahydropyridin-4-yl)-[1,2,4]triazolo[4,3-a]pyridine (28-1). To a solution of 8-bromo-[1,2,4]triazolo[4,3-a]pyridine (0.500 g, 2.52 mmol) in dioxane:water (10:1, 11 mL) was added tert-butyl 4-(4,4,5,5-tetramethyl-1,3,2-dioxaborolan-2-yl)-3,6-dihydropyridine-1(2H)-carboxylate (1.17 g, 3.79 mmol), K₂CO₃ (0.698 g, 5.05 mmol) and Pd(PPh₃)₄ (0.146 g, 0.126 mmol) then heated to reflux overnight. To this was then added brine (50 mL) then extracted with EtOAc (50 mL×3). The organic layer was dried with Na₂SO₄, filtered, then concentrated and purified via silica gel chromatography eluting 0-100% EtOAc in hexanes followed by 0-20% MeOH in CH₂Cl₂. The product was isolated as a pink solid. The product was then concentrated and dissolved in CH₂Cl₂ (10 mL) and then added TFA (3 mL) and stirred at room temperature for ˜1 h. To the reaction mixture was added sat. NaHCO₃ (aq (50 mL) then extracted with CH₂Cl₂ (50 mL×3). The organic layers were isolated and concentrated to yield 28-1 as a yellow solid. Yield=0.352 g (69%). M/Z: 201 [M+H]⁺.

Step 2: 8-(Piperidin-4-yl)-5,6,7,8-tetrahydro-[1,2,4]triazolo[4,3-a]pyridine (28-2). To a solution of 28-1 (0.352 g, 1.76 mmol) in MeOH (10 mL) was added palladium on carbon (5% wt. loading, 18 mg, 0.176 mmol) and degassed under vacuum for ˜15 mins. The reaction mixture was then placed under hydrogen atmosphere and stirred at room temperature for ˜2 hrs. This was then filtered through celite to remove palladium. The filtrate was concentrated to provide 28-2, which was used in the next step without further purification. Yield=0.355 g (99%). M/Z: 203 [M+H]⁺.

Step 3: 8-(1-(3-Bromopyridin-2-yl)piperidin-4-yl)-5,6,7,8-tetrahydro-[1,2,4]triazolo[4,3-a]pyridine (28-3). To a solution of 28-2 obtained in Step 2 (0.355 g, 1.76 mmol) in DMF (5 mL) was added 3-bromo-2-chloropyridine (0.506 g, 2.64 mmol) and heated at 140° C. overnight. The reaction mixture was then concentrated and resuspended in MeOH (20 mL), then filtered to remove solids. The filtrate was again concentrated then purified via C18 reverse phase chromatography eluting 5-95% ACN (+0.1% NH₄OH) in H₂O (+0.1% NH₄OH) to yield 28-3. Yield=0.201 g (32%). M/Z: 357, 359 [M+H]⁺.

Step 4: 8-(1-(6′-fluoro-[3,3′-bipyridin]-2-yl)piperidin-4-yl)-5,6,7,8-tetrahydro-[1,2,4]triazolo[4,3-a]pyridine (Compound 28). To a solution of 28-3 (0.201 g, 0.563 mmol) in dioxane:water (10:1, 11 mL) was added K₂CO₃ (0.155 g, 1.12 mmol) and (6-fluoropyridin-3-yl)boronic acid (0.119 g, 0.844 mmol). To the reaction mixture was then added Pd(PPh₃)₄ (0.005 g, 0.004 mmol) and heated to reflux overnight. This was then concentrated and resuspended in MeOH (20 mL), then filtered to remove solids. The filtrate was concentrated then purified via prep HPLC via C18 reverse phase chromatography eluting 5-95% ACN (+0.1% NH₄OH) in H₂O (+0.1% NH₄OH) to yield Compound 28. Yield=0.01 g (5%). M/Z: 375 [M+H]⁺.

The following compounds were prepared similarly to Examples 4-8 with appropriate reagents and substrates at different steps.

| Compound no. | MS (M + H)⁺ |
|---|---|
| 25 | 351 |

### Example 9. Preparation of 5-(1-(6′-fluoro-6-methyl-[3,3′-bipyridin]-2-yl)piperidin-4-yl)-4-methyl-4H-1,2,4-triazol-3-amine (Compound 4)

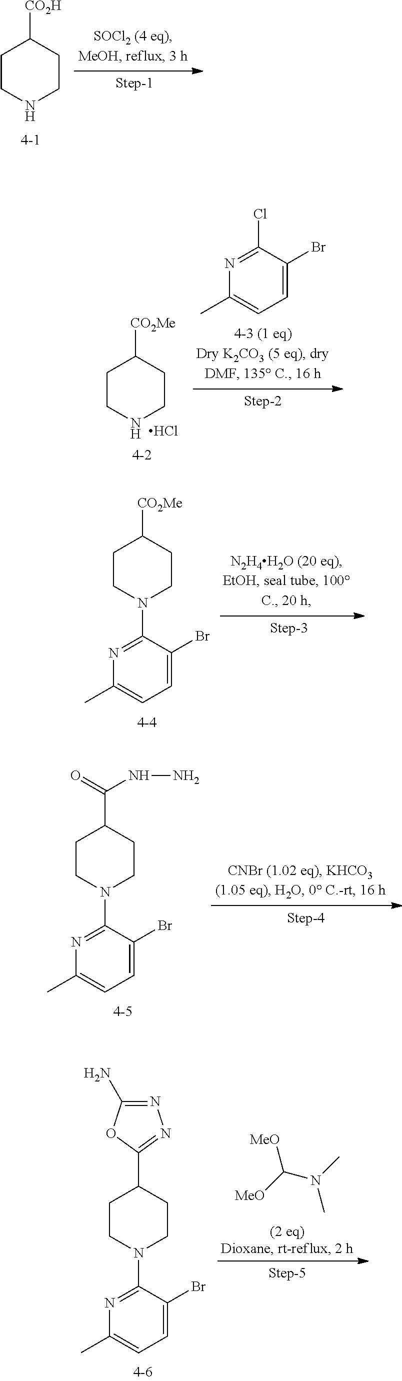

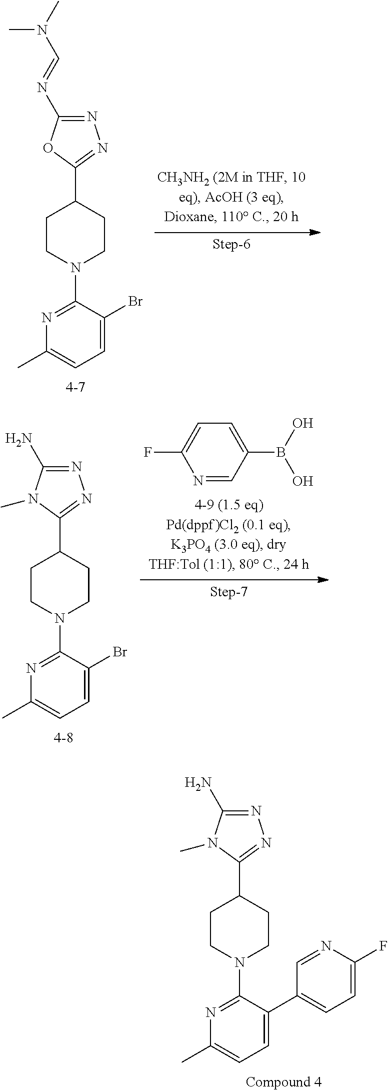 [SMILES: Cc1ccc(Br)c(N2CCC(c3nnc(/N=C/N(C)C)o3)CC2)n1 ; Cc1ccc(Br)c(N2CCC(c3nnc(N)n3C)CC2)n1 ; OB(O)c1ccc(F)nc1 ; Cc1ccc(-c2ccc(F)nc2)c(N2CCC(c3nnc(N)n3C)CC2)n1]

Step 1: methyl piperidine-4-carboxylate hydrochloride (4-2). To a stirred solution of piperidine-4-carboxylic acid (4-1, 10 g, 77.39 mmol) in MeOH (100 mL) was added thionyl chloride (22.4 mL, 309.56 mol) at 0° C. and reaction mixture was refluxed for 3 h. The progress of the reaction was monitored by TLC. The reaction mixture was concentrated under reduced pressure. The crude solid was washed with diethyl ether and dried under reduced pressure to afford 4-2 as an off-white solid (12 g, 88.2% yield). ¹H-NMR was consistent with the proposed structure.

Step 2: methyl 1-(3-bromo-6-methylpyridin-2-yl)piperidine-4-carboxylate (4-4). To a stirred suspension of 4-2 (2 g, 13.98 mmol) in dry DMF (40 mL) were added dry K₂CO₃ (9.65 g, 69.9 mmol) at room temperature stirred for 10 min. Then 3-bromo-2-chloro-6-methylpyridine (4-3, 2.88 g, 13.98 mmol) was added at room temperature and the contents were heated at 135° C. for 16 h. The progress of the reaction was monitored by TLC and LCMS. The reaction mixture filtered, the filtrate was extracted with ice-cold water (100 mL) and EtOAc (3×100 mL). The combined organic layers were washed with brine solution, dried over Na₂SO₄ and concentrated under reduced pressure. The crude compound was purified by column chromatography to obtain 4-4 (2 g, 45.76% yield). ¹H-NMR was consistent with the proposed structure.

Step 3: 1-(3-bromo-6-methylpyridin-2-yl)piperidine-4-carbohydrazide (4-5). To a seal tube containing suspension of 4-4 (1.2 g, 3.19 mmol) in EtOH (20 mL) was added 80% hydrazine hydrate (3.19 mL, 63.8 mmol) at room temperature. The contents were heated at 100° C. for 20 h. The progress of reaction was monitored by TLC, after completion of the reaction the solvent removed under reduced pressure. The crude compound was purified by column chromatography to afford 4-5 as off-white solid (1.1 g, 91.6% yield). ¹H-NMR was consistent with the proposed structure.

Step 4: 5-(1-(3-bromo-6-methylpyridin-2-yl)piperidin-4-yl)-1,3,4-oxadiazol-2-amine (4-6). To a stirred suspension of 4-5 (1 g, 3.19 mmol) in water (20 mL) were added aq. KHCO₃ (0.35 g, dissolved in 5 mL water, 3.34 mmol) and CNBr (0.35 g, dissolved in 8 mL water, 3.25 mmol) at 0° C. The reaction mixture stirred at room temperature for 16 h. The progress of reaction was monitored by LCMS. The reaction mixture was diluted with water (50 mL), solid was filtered and the obtained solid was washed with water. The resulting solid was dried under reduced pressure to afford 4-6 as white solid (0.9 g, 83.4% yield). ¹H-NMR was consistent with the proposed structure.

Step 5: N′-(5-(1-(3-bromo-6-methylpyridin-2-yl)piperidin-4-yl)-1,3,4-oxadiazol-2-yl)-N,N-dimethylformimidamide (4-7). To a stirred suspension of 4-6 (0.9 g, 2.66 mmol) in dry 1,4-dioxane (12 mL) was added DMF-DMA (0.70 mL, 5.32 mmol) at room temperature. The contents were refluxed for 2 h. Progress of reaction was monitored by TLC. After completion of the reaction, the reaction mass was diluted with EtOAc (50 mL) and washed with ice-cold water (20 mL) and brine solution, the organic layer was dried over Na₂SO₄ and concentrated under reduced pressure to afford 4-7 as pale yellow liquid (1.02 g, 98.07% yield). The crude compound was analyzed by LCMS (96.17%) and used as such for the next step.

Step 6: 5-(1-(3-bromo-6-methylpyridin-2-yl)piperidin-4-yl)-4-methyl-4H-1,2,4-triazol-3-amine (4-8). To a seal tube containing suspension of 4-7 (1.02 g, 2.59 mmol) in dry 1,4-dioxane (15 mL) were added methyl amine (12.97 mL, 2 M in THF, 25.9 mmol) and acetic acid (0.44 mL, 7.77 mmol) at room temperature. The seal tube was closed under nitrogen and stirred at 110° C. for 20 h. Progress of reaction was monitored by TLC and LCMS. After completion of the reaction, the reaction mixtures was concentrated under reduced pressure, obtained crude was basified with aq. NaOH solution, extracted with 10% MeOH in EtOAc (3×100 mL). The combined organic layers were dried over Na₂SO₄ and concentrated under reduced pressure. The crude compound was purified by grace column chromatography to afford 4-8 as pale yellow solid (0.15 g, 16.48% yield). ¹H-NMR was consistent with the proposed structure, and the isomer was confirmed by NOE.

Step 7: 5-(1-(6′-fluoro-6-methyl-[3,3′-bipyridin]-2-yl)piperidin-4-yl)-4-methyl-4H-1,2,4-triazol-3-amine (Compound 4). To a sealed tube containing 4-8 (0.1 g, 0.28 mmol) in degassed dry THF:Toluene (1:1, v/v, 12 mL), were added (6-fluoropyridin-3-yl)boronic acid (4-9, 0.06 g, 0.42 mmol), K₃PO₄ (0.18 g, 0.85 mmol) and Pd(dppf)₂Cl₂ (0.02 g, 0.028 mmol) under degassed condition. The sealed tube closed under argon atmosphere and heated at 80° C. for 24 h. The progress of reaction was monitored by TLC. After completion of the reaction, the reaction mass was filtered through celite pad, and the pad was washed with 10% MeOH in DCM and the obtained filtrate was concentrated under reduced pressure. The crude compound was purified by Prep-HPLC using 0.1% formic acid to afford Compound 4 (formate salt) as a white solid (32 mg, 30.76% yield). ¹H NMR (400 MHz, DMSO-d₆): δ 8.42 (s, 1H), 8.22 (td, J=2.4, 8.2 Hz, 1H), 8.13 (s, 0.74H, formate), 7.51 (d, J=7.6 Hz, 1H), 7.26 (dd, J=2.4, 8.6 Hz, 1H), 6.90 (d, J=7.6 Hz, 1H), 5.93 (br-s, 2H, D2O-exchangeable), 3.40 (d, J=12.8 Hz, 2H), 3.26 (s, 3H), 2.80-2.61 (m, 3H), 2.39 (s, 3H), 1.74 (d, J=11.6 Hz, 2H), 1.62-1.50 (m, 2H).

### Example A-1: Parenteral Pharmaceutical Composition

To prepare a parenteral pharmaceutical composition suitable for administration by injection (subcutaneous, intravenous), 1-100 mg of a water-soluble salt of a compound Formula (A), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection

### Example A-2: Oral Solution

To prepare a pharmaceutical composition for oral delivery, a sufficient amount of a compound of Formula (A), or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound of Formula (A), or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 10-500 mg of a compound of Formula (A), or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound of Formula (A), or a pharmaceutically acceptable salt thereof, is placed into Size 4 capsule, or size 1 capsule (hypromellose or hard gelatin) and the capsule is closed.

### Example B-1: Glutaminyl-Peptide Cyclotransferase Like (OPCTL) Protein Inhibition Assay

Glutaminyl-peptide cyclotransferase like (QPCTL) protein activity was determined using L-glutamine-7-amido-4-methylcoumarin (Gln-AMC, Santa Cruz Biotechnology) as a substrate. A second enzyme, pyroglutamyl aminopeptidase (pGAPase, Qiagen), was included in the assay to release the AMC fluorophore following cyclization; appropriate controls were run to ensure that inhibitors were not inhibiting the auxiliary enzyme. Inhibitors were added at various concentrations to buffer (final concentrations: 50 mM MES pH 7.0, 10 μM ZnCl2, 0.2 U/mL pGAPase, Gln-AMC at its Km) and the reaction initiated by the addition of QPCTL. Reactions became linear after a 10-minute equilibration period at 30° C. (to accumulate pGlu-AMC intermediate), and were monitored for an additional 10 minutes.

Table A provides QPCTL inhibitory activity of illustrative compounds, where A means IC₅₀ is <1 μM; B means IC₅₀ is between 1 to 10 μM; C means IC₅₀ is between 10 to 50 PM; D means IC₅₀ is >50 PM.

TABLE A
Representative QPCTL Activity

| Compd No. | QPCTL IC₅₀ |
|---|---|
| 1 | A |
| 2 | A |
| 3 | A |
| 4 | A |
| 5 | A |
| 6 | A |
| 7 | A |
| 10 | A |
| 11 | C |
| 12 | B |
| 13 | B |
| 14 | B |
| 15 | A |
| 16 | C |
| 17 | A |
| 18 | B |
| 19 | B |
| 20 | B |
| 21 | D |
| 22 | C |
| 23 | C |
| 24 | B |
| 25 | D |
| 26 | B |
| 27 | C |
| 28 | A |

The examples and embodiments described herein are for illustrative purposes only and various modifications or changes suggested to persons skilled in the art are to be included within the spirit and purview of this application and scope of the appended claims.

We claim:

1. A compound of Formula (A): 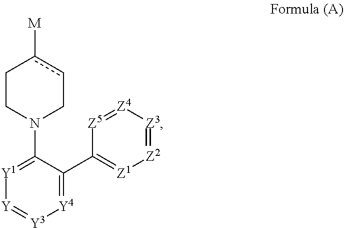
or a pharmaceutically acceptable salt, or solvate thereof, wherein:
M is 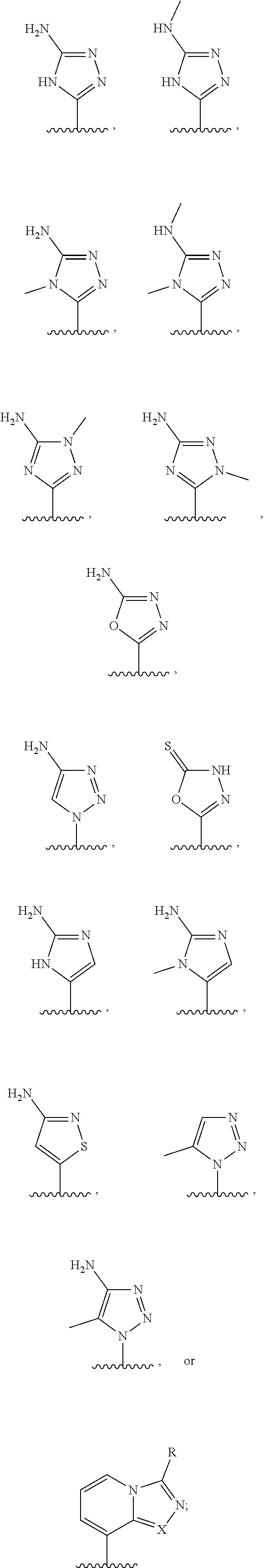
X is N or CH;
R is H, NH₂, or NHCH₃; [symbol]
is a single bond or a double bond;
Y¹ is N or CR¹; Y² is N or CR², Y³ is N or CR³; and Y⁴ is N or CR⁴;
Z¹ is N or CR⁵; Z² is N or CR⁶; Z³ is N or CR⁷; Z⁴ is N or CR⁸; and Z⁵ is N or CR⁹;
R¹, R², R³, R⁴, R⁵, R⁶, R⁷, R⁸, and R⁹ are each independently hydrogen, halogen, unsubstituted or substituted C₁-C₆ alkyl, unsubstituted or substituted C₂-C₆ alkenyl, unsubstituted or substituted C₂-C₆ alkynyl, unsubstituted or substituted C₁-C₆ fluoroalkyl, unsubstituted or substituted C₁-C₆ heteroalkyl, unsubstituted or substituted C₃-C₆ cycloalkyl, unsubstituted or substituted 4- to 6-membered heterocycloalkyl, —CN, —OR¹⁰, —CO₂R¹⁰, —C(═O)N(R¹⁰)₂, —N(R¹⁰)₂, —NR¹C(═O)R¹¹, —SR¹⁰, —S(═O)R¹¹, —SO₂R¹¹, —SO₂N(R¹⁰)₂, or —N(R¹⁰)SO₂R¹¹;
each R¹⁰ is independently selected from hydrogen, unsubstituted or substituted C₁-C₆ alkyl, unsubstituted or substituted C₁-C₆ heteroalkyl, unsubstituted or substituted C₃-C₆ cycloalkyl, 4- to 6-membered heterocycloalkyl;
or two R¹⁰ on the same N atom are taken together with the N atom to which they are attached to form an unsubstituted or substituted N-containing 4- to 6-membered heterocycloalkyl; and
each R¹¹ is independently selected from unsubstituted or substituted C₁-C₆ alkyl, unsubstituted or substituted C₁-C₆ heteroalkyl, unsubstituted or substituted C₃-C₆ cycloalkyl, 4- to 6-membered heterocycloalkyl;
wherein each substituted alkyl, substituted alkenyl, substituted alkynyl, substituted fluoroalkyl, substituted heteroalkyl, substituted cycloalkyl, and substituted heterocycloalkyl is substituted with one or more R^{s} groups independently selected from the group consisting of halogen, C₁-C₆ alkyl, monocyclic carbocycle, monocyclic heterocycle, —CN, —OR¹², —CO₂R¹², —C(═O)N(R¹²)₂, —N(R¹²)₂, —NR¹²C(═O)R¹³, —SR¹², —S(═O)R¹³, —SO₂R¹³, —SO₂N(R¹²)₂, or —N(R¹²)SO₂R¹³;
each R¹² is independently selected from hydrogen, C₁-C₆ alkyl, C₁-C₆ heteroalkyl, C₃-C₆ cycloalkyl, 4- to 6-membered heterocycloalkyl, phenyl, 5-membered heteroaryl and 6-membered heteroaryl; or two R¹² groups are taken together with the N atom to which they are attached to form a N-containing 4- to 6-membered heterocycloalkyl;
each R¹³ is independently selected from C₁-C₆ alkyl, C₁-C₆ heteroalkyl, C₃-C₆ cycloalkyl, 4- to 6-membered heterocycloalkyl, phenyl, 5-membered heteroaryl and 6-membered heteroaryl;
wherein 0, 1, or 2 of Y¹, Y², Y³, and Y⁴ are N; and
wherein 0, 1, or 2 of Z¹, Z², Z³, Z⁴, and Z⁵ are N.

2. The compound of claim 1, wherein the compound is a compound of Formula (A1): 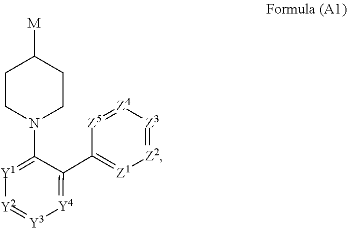
or a pharmaceutically acceptable salt, or solvate thereof.

3. The compound of claim 1, wherein the compound is a compound of Formula (A2): 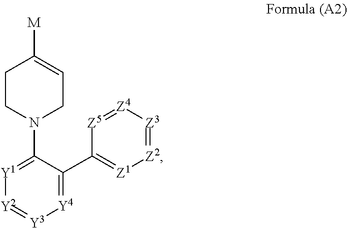
or a pharmaceutically acceptable salt, or solvate thereof.

4. The compound of any one of claims 1-3, or a pharmaceutically acceptable salt, or solvate thereof, wherein:
0 or 1 of Y¹, Y², Y³, and Y⁴ is N.

5. The compound any one of claims 1-4, or a pharmaceutically acceptable salt, or solvate thereof, wherein:
0 or 1 of Z¹, Z², Z³, Z⁴, and Z⁵ is N.

6. The compound of any one of claims 1-5, or a pharmaceutically acceptable salt, or solvate thereof, wherein:
0 or 1 of Y¹, Y², Y³, and Y⁴ is N; and
0 or 1 of Z¹, Z², Z³, Z⁴, and Z⁵ is N.

7. The compound of claim 1, wherein the compound is a compound of Formula (B): 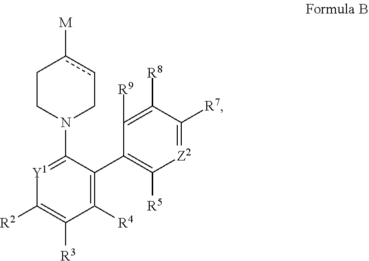
or a pharmaceutically acceptable salt, or solvate thereof.

8. The compound of claim 7, wherein the compound is a compound of Formula (B1): 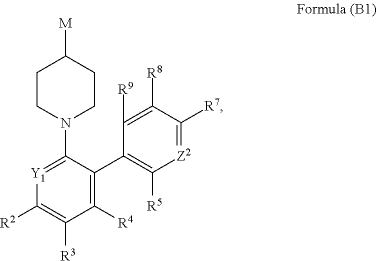
or a pharmaceutically acceptable salt, or solvate thereof.

9. The compound of claim 7, wherein the compound is a compound of Formula (B2): 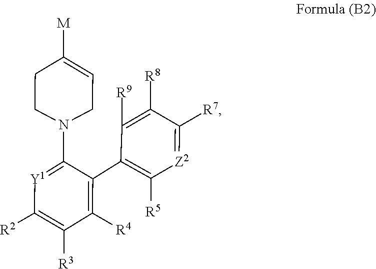
or a pharmaceutically acceptable salt, or solvate thereof.

10. The compound of claim 1, wherein the compound is a compound of Formula (C): 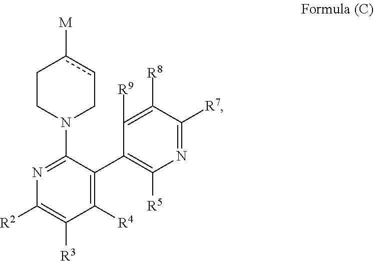
or a pharmaceutically acceptable salt, or solvate thereof.

11. The compound of claim 10, wherein the compound is a compound of Formula (C1): 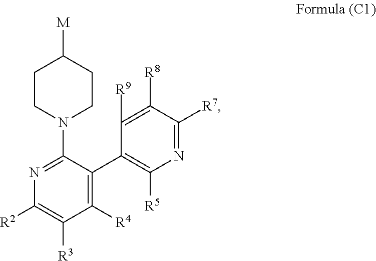
or a pharmaceutically acceptable salt, or solvate thereof.

12. The compound of claim 10, wherein the compound is a compound of Formula (C2): 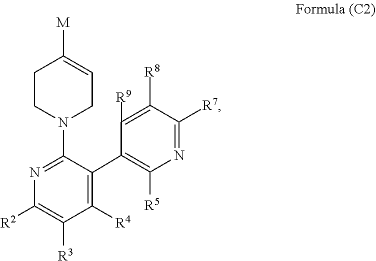
or a pharmaceutically acceptable salt, or solvate thereof.

13. The compound of any one of claims 1-12, or a pharmaceutically acceptable salt, or solvate thereof, wherein:
M is

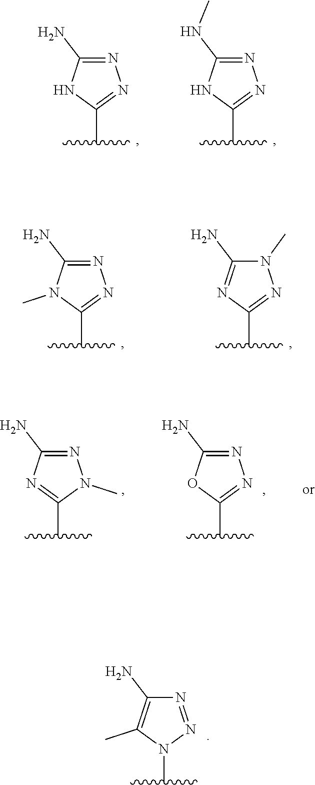

14. The compound of any one of claims 1-13, or a pharmaceutically acceptable salt, or solvate thereof, wherein:
M is

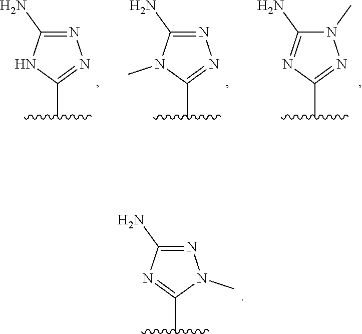

15. The compound of any one of claims 1-14, or a pharmaceutically acceptable salt, or solvate thereof, wherein:
M is

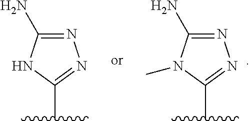

16. The compound of claim 11, wherein the compound is a compound of Formula (C1-a) or Formula (C1-c): 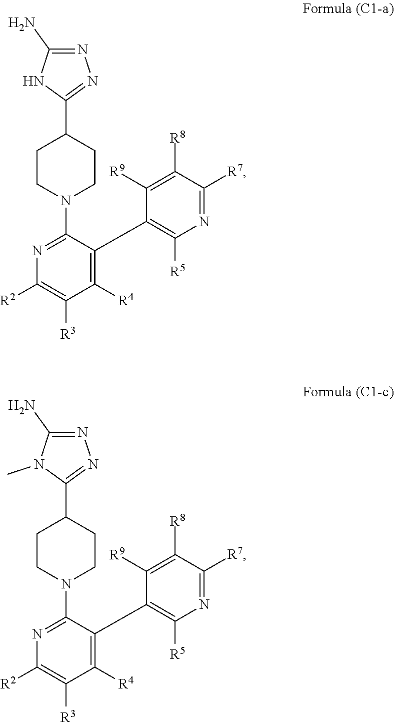
or a pharmaceutically acceptable salt, or solvate thereof.

17. The compound of any one of claims 1-12, or a pharmaceutically acceptable salt, or solvate thereof, wherein:
M is

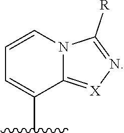

18. The compound of claim 17, or a pharmaceutically acceptable salt, or solvate thereof, wherein:
X is N.

19. The compound of claim 17, or a pharmaceutically acceptable salt, or solvate thereof, wherein:
X is CH.

20. The compound of any one of claims 17-19, or a pharmaceutically acceptable salt, or solvate thereof, wherein:
R is NH₂.

21. The compound of any one of claims 17-19, or a pharmaceutically acceptable salt, or solvate thereof, wherein:
R is H.

22. The compound of claim 17, or a pharmaceutically acceptable salt, or solvate thereof, wherein:
M is

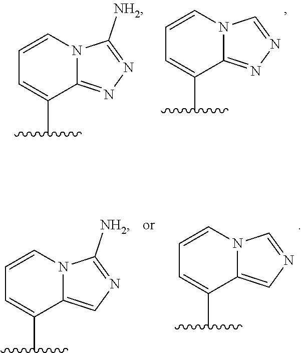

23. The compound of claim 17, or a pharmaceutically acceptable salt, or solvate thereof, wherein:
M is

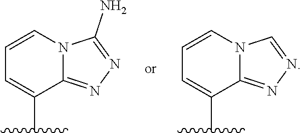 [SMILES: *c1cccn2c(N)nnc12 ; "or" ; *c1cccn2cnnc12]

24. The compound of claim 11, wherein the compound is a compound of Formula (C1-o): 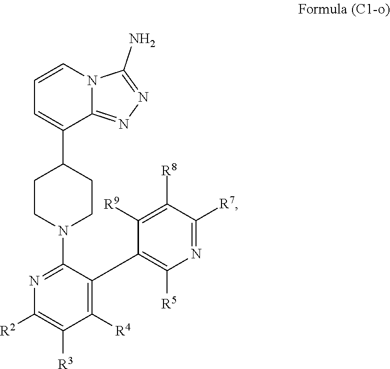
or a pharmaceutically acceptable salt, or solvate thereof.

25. The compound of claim 12, wherein the compound is a compound of Formula (C2-o) or (C2-p): 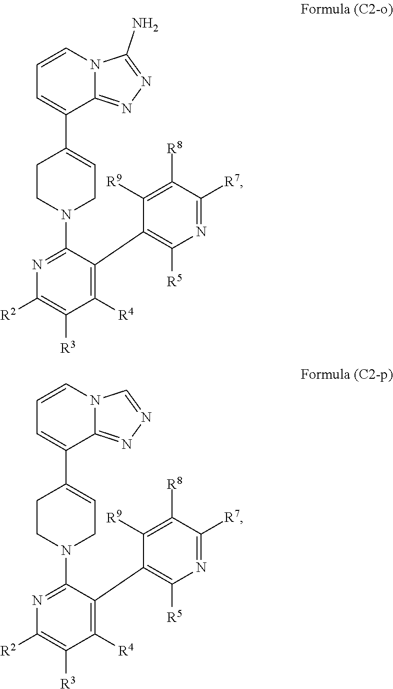
or a pharmaceutically acceptable salt, or solvate thereof.

26. The compound of any one of claims 1-25, or a pharmaceutically acceptable salt, or solvate thereof, wherein:
R², R³, R⁴, R⁵, R⁷, R⁸, and R⁹ are each independently hydrogen, halogen, —CN, —OR¹⁰, —N(R¹⁰)₂, C₁-C₆ alkyl, or C₁-C₆ fluoroalkyl.

27. The compound of any one of claims 1-26, or a pharmaceutically acceptable salt, or solvate thereof, wherein:
R², R³, R⁴, R⁵, R⁷, R⁸, and R⁹ are each independently hydrogen, —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃.

28. The compound of any one of claims 1-27, or a pharmaceutically acceptable salt, or solvate thereof, wherein:
R², R³, R⁴, R⁵, R⁷, R⁸, and R⁹ are each independently hydrogen, —F, or —CH₃.

29. The compound of claim 1, or a pharmaceutically acceptable salt, or solvate thereof, wherein the compound is a compound of Formula (D): 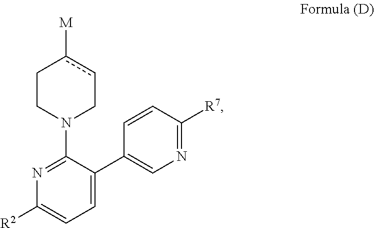
or a pharmaceutically acceptable salt, or solvate thereof; wherein:
R² and R⁷ are each independently hydrogen, halogen, —CN, —OR¹⁰, —N(R¹⁰)₂, C₁-C₆ alkyl, or C₁-C₆ fluoroalkyl.

30. The compound of claim 29, wherein the compound is a compound of Formula (D1): 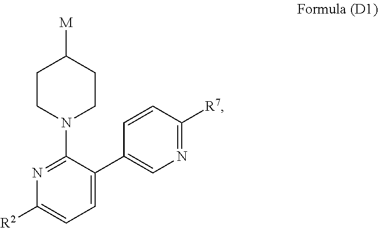
or a pharmaceutically acceptable salt, or solvate thereof.

31. The compound of claim 29, wherein the compound is a compound of Formula (D2): 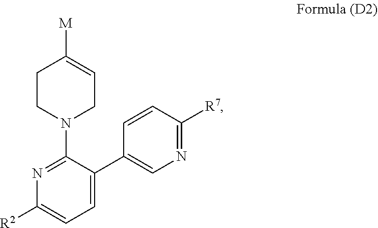
or a pharmaceutically acceptable salt, or solvate thereof.

32. The compound of any one of claims 29-31, or a pharmaceutically acceptable salt, or solvate thereof, wherein:
R² is hydrogen, —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃;
R⁷ is hydrogen, —F, —Cl, —CN, —OCH₃, —OCH₂CH₃, —OCH₂CH₂OCH₃, —NH₂, —NHCH₃, —N(CH₃)₂, —CH₃, —CH₂CH₃, —CH(CH₃)₂, —C(CH₃)₃, —CHF₂, or —CF₃.

33. The compound of any one of claims 29-32, or a pharmaceutically acceptable salt, or solvate thereof, wherein:
R² is hydrogen, —F, or —CH₃;
R⁷ is hydrogen, —F, —Cl, —CN, —OCH₃, —OCH₂CH₂OCH₃, or —NH₂.

34. The compound of claim 1, selected from: 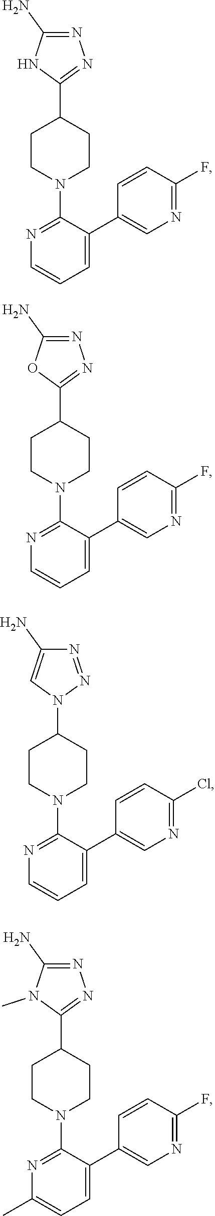 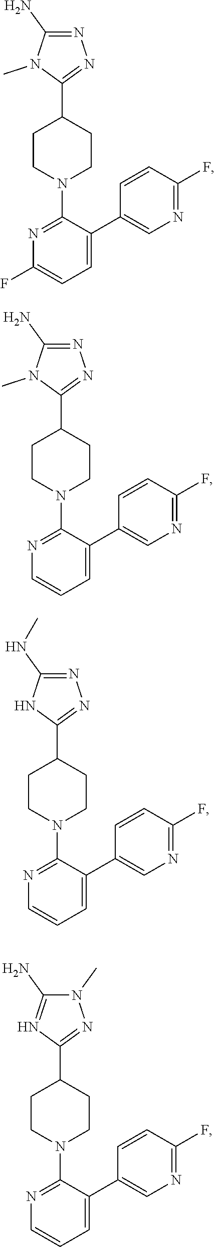 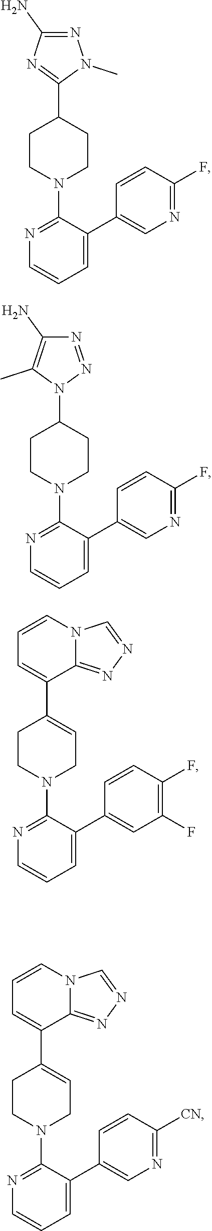 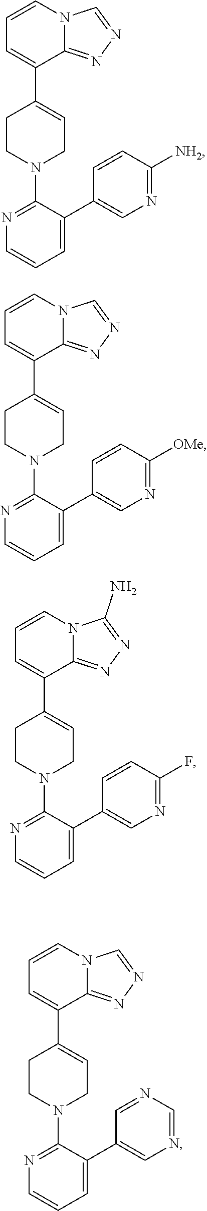 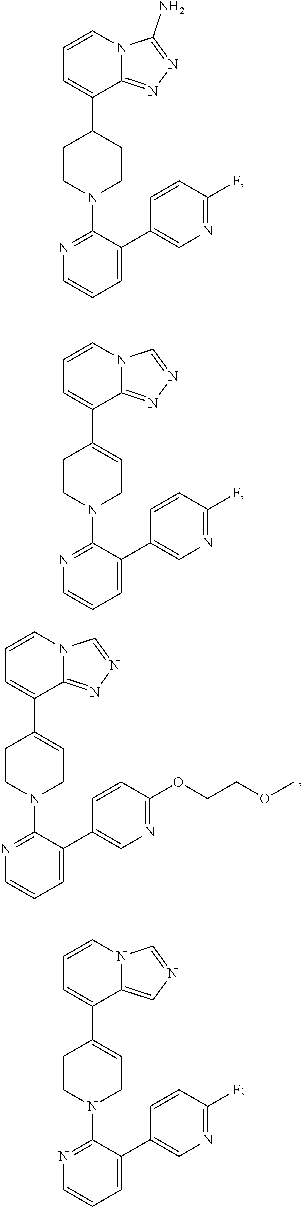
or a pharmaceutically acceptable salt, or solvate thereof.

35. A pharmaceutical composition comprising the compound of any one of claims 1-34, or a pharmaceutically acceptable salt, or solvate thereof, and a pharmaceutically acceptable excipient.

36. A method of treating a disease of condition amenable to treatment with a glutaminyl-peptide cyclotransferase like (QPCTL) inhibitor, the method comprising administering a compound of any one of claims 1-34, or a pharmaceutically acceptable salt, or solvate thereof to a subject in need thereof, or the pharmaceutical composition of claim 35.

37. The method of claim 36, wherein the disease or condition is a cancer.

38. The method of claim 37, wherein the cancer is a leukemia or lymphoma.

39. The method of claim 38, wherein the leukemia or lymphoma is acute myeloid leukemia (AML), chronic myeloid leukemia (CMIL), acute lymphocytic leukemia (ALL), chronic lymphocytic leukemia (CLL), non-Hodgkin's lymphoma (NHL).

40. The method of claim 39, wherein the non-Hodgkin's lymphoma is a B-cell lymphoma.

41. The method of claim 40, wherein the B-cell lymphoma is selected from the group consisting of Burkitt lymphoma, hairy cell lymphoma (HCL), Waldenstrom macroglobulinemia, chronic lymphocytic leukemia/small lymphocytic lymphoma (CLL/SLL), diffuse large B cell lymphoma (DLBCL), B cell chronic lymphocytic leukemia (B-CLL), mantle cell lymphoma (MCL), follicular lymphoma (FL), marginal zone lymphoma (MZL), and pre-B acute lymphoblastic leukemia (pre-B ALL).

42. The method of claim 37, wherein the cancer is selected from the group consisting of: multiple myeloma (MM), ovarian cancer, gliomas, colon cancer, breast cancer, bladder cancer, gastric cancer, esophageal cancer, small cell lung cancer (SCLC), non-small cell lung cancer (NSCLC), head and neck squamous cell cancer, mesothelioma, melanoma, glioma, glioblastoma, and pancreatic neuroendocrine tumors.

43. The method of claim 37, wherein the cancer is selected from the group consisting of: basal cell carcinoma, squamous cell carcinoma, renal cell carcinoma, invasive ductal carcinoma, adenocarcinoma, Merkel cell carcinoma, skin cancer, lung cancer, breast cancer, prostate cancer, colorectal cancer, soft tissue sarcoma, osteosarcoma, Ewing's sarcoma, chrondrosarcoma, myeloma, or multiple myeloma.

44. The method of claim 36, wherein the disease or condition is an inflammatory disease, autoimmune disease, allergic inflammatory disease, or neurodegenerative disease.

45. The method of claim 44, wherein the disease or condition is rheumatoid arthritis, inflammatory bowel disease, multiple sclerosis, psoriasis, psoriatic arthritis, atopic dermatitis, severe asthma, allergic rhinitis and rhinosinusitis, nasal polyposis, atherosclerosis, pulmonary arterial hypertension, non-alcoholic steatohepatitis, chronic obstructive pulmonary disease, idiopathic pulmonary fibrosis, endometriosis, Alzheimer's disease and related dementias, Parkinson's disease, or Huntington's disease.
